(19) United States
(12) Patent Application Publication
(10) Pub. No.: US 2021/0222175 A1
(43) Pub. Date: Jul. 22, 2021

(54) TARGETING GENE AMPLIFICATION IN CANCER USING TRIPLEX FORMATION AS A THERAPEUTIC STRATEGY

(71) Applicant: Yale University, New Haven, CT (US)

(72) Inventor: Faye A. Rogers, Norwalk, CT (US)

(73) Assignee: Yale University, New Haven, CT (US)

(21) Appl. No.: 17/160,050

(22) Filed: Jan. 27, 2021

Related U.S. Application Data
(63) Continuation-in-part of application No. 16/683,205, filed on Nov. 13, 2019.
(60) Provisional application No. 62/767,279, filed on Nov. 14, 2018.

(51) Int. Cl. C12N 15/113; A61P 35/00; A61K 47/69

(52) U.S. Cl. CPC C12N 15/1135; A61K 47/6937; A61P 35/00

(57) ABSTRACT

Disclosed herein are methods and agents for the treatment of cancer using p53-independent apoptosis to reduce the number of cancer cells that have an amplified HER2 gene, such as p53-depleted or p53-mutated cancer cells that have an amplified HER2 gene. Also disclosed herein are methods and agents for the treatment of HER2-positive cancer in individuals with Li-Fraumeni Syndrome.

### RELATED APPLICATIONS

This application is a continuation-in-part application of U.S. application Ser. No. 16/683,205, filed Nov. 13, 2019, which claims the benefit under 35 U.S.C. § 119(e) of U.S. provisional application Ser. No. 62/767,279, filed Nov. 14, 2018. The teachings and text of the aforementioned applications are incorporated by reference herein in their entirety.

### GOVERNMENT LICENSE RIGHTS

This invention was made with government support under GM126211 and CA185192 awarded by National Institutes of Health. The government has certain rights in the invention.

### BACKGROUND

Amplification of genes involved in normal cell growth and survival pathways drives oncogenesis in a broad spectrum of cancers^{1,2}, ultimately affecting tumor progression and clinical outcome^{3,4}. Several drugs have been developed to inhibit the oncogenic activity of amplified driver genes⁵. The majority of these cancer therapeutics target the overexpressed protein products and their clinical efficacy is often hampered by drug resistance^{6,7}.

### SUMMARY

Described herein is a novel therapeutic method for the treatment of cancers that are characterized by gene amplification, and, in one embodiment specifically, the treatment of cancers that are characterized by HER2 gene amplification. In the method, manipulation of the DNA damage response with triplex-forming oligonucleotides (TFOs) drives p53-independent tumor-specific induction of apoptosis. The method described is particularly applicable to p53-mutant or deficient cancers, which are often aggressive and resistant to traditional chemotherapeutic drugs. This provides a new and specific approach in targeted cancer therapy, which can have enormous impact on the field of precision medicine.

Described herein is a method of inducing target-specific DNA damage in cancer cells in which a gene is amplified (present in multiple copies) and triplex forming oligonucleotides (TFOs) that are useful in the method. The amplified gene (gene present in multiple copies) that serves as a target of TFOs can be a cancer-driver (cancer-promoting) gene or a passenger gene (e.g., a gene that does not drive or promote cancer/comprises mutation(s) that do not directly drive cancer initiation and progression). In one embodiment of the method, cancer cells comprising an amplified gene are contacted with triplex forming oligonucleotides (TFOs) that are targeted to a polypurine site (referred to as a targeted amplified gene locus) within the amplified gene, under conditions under which the TFOs enter the cancer cells, recognize (such as by binding) the polypurine site and induce DNA damage at the targeted amplified gene locus. DNA damage can occur in many forms, such as, but not limited to, DNA strand breaks (double strand or single strand), damage by alkylation of bases, oxidation, hydrolysis and cross linking. In one embodiment of the method, cancer cells in which a cancer-promoting gene, such as a gene linked to a signaling pathway, is amplified are contacted with triplex forming oligonucleotides (TFOs) that are targeted to a polypurine site (referred to as a targeted amplified gene locus) within the amplified cancer-promoting gene, under conditions under which the TFOs enter the cancer cells, recognize (such as by binding), the polypurine site and induce DNA damage at the targeted amplified gene locus. Results show that triplex-induced DNA damage can activate apoptosis, such as p53-independent apoptosis, and that the mechanism of action for HER2-targeted TFOs is independent of HER2 cellular function. DNA damage is, for example, DNA double strand breaks, such as copy number dependent DNA double strand breaks. In a specific example, the method is a method of inducing target-specific DNA damage in cancer cells in which a HER2 gene is amplified, the method comprising contacting (a) cancer cells in which a HER2 gene is amplified with (b) triplex forming oligonucleotides (TFOs) targeted to a polypurine site, referred to as a targeted amplified gene locus, within the amplified HER2 gene, under conditions under which the TFOs enter the cancer cells, bind the polypurine site and induce DNA damage at the targeted amplified gene locus. In one embodiment, DNA damage is copy-number dependent DNA double strand breaks and DNA damage is sufficient to activate apoptosis (e.g., p53-independent apoptosis) in cancer cells in which a HER2 gene is amplified. In some embodiments, the cancer cells in which a HER2 gene is amplified are HER2-amplified breast cancer cells or ovarian HER2-amplified breast cancer cells. The cancer cells are mammalian cells, generally human cells. The population of cells can be in an individual, such as in a human tissue, fluid or organ.

In some embodiments, the polypurine target site (also referred to as a targeted amplified gene locus) is/comprises SEQ ID NO: 1, SEQ ID NO: 2, SEQ ID NO: 5 or SEQ ID NO: 6. In some embodiments, the TFOs comprise at least one of the following: a nucleotide sequence identical to SEQ ID NO:3; a nucleotide sequence identical to SEQ ID NO:4; a nucleotide sequence identical to SEQ ID NO:7; a nucleotide sequence identical to SEQ ID NO:8; a nucleotide at least 90% identical to SEQ ID NO: 3; a nucleotide sequence at least 90% identical to SEQ ID NO: 4; a nucleotide sequence at least 90% identical to SEQ ID NO; 7 and a nucleotide at least 90% identical to SEQ ID NO: 8.

The polypurine site can be located in a variety of locations within an amplified cancer-promoting gene, such as in an amplified HER2 gene. For example, the polypurine site can be in the promoter region of the amplified cancer-promoting gene, such as in the promoter region of amplified HER2 gene; in a coding region of the amplified cancer-promoting gene, such as in the coding region of amplified HER2 gene; or in an intron of the amplified cancer-promoting gene, such as in an intron of amplified HER2 gene.

In another embodiment, the invention is a method of reducing, in a population of cells comprising cancer cells, the number of cancer cells in which a cancer-promoting gene is amplified. In the method, triplex-induced DNA damage is sufficient to activate p53-independent apoptosis. In the method, a population of cells comprising an amplified cancer-promoting gene is contacted with triplex forming oligonucleotides (TFOs) targeted to a polypurine target site (referred to as a targeted amplified gene locus) in the amplified cancer-promoting gene, under conditions under which the TFOs enter the cancer cells in sufficient quantity to induce apoptosis. DNA damage is, for example, DNA double strand breaks, such as copy number dependent DNA double strand breaks. In a specific example, the method is a method of inducing target-specific DNA damage in cancer cells in which a HER2 gene is amplified, the method comprising contacting (a) cancer cells in which a HER2 gene is amplified with (b) triplex forming oligonucleotides (TFOs) targeted to a polypurine site, referred to as a targeted amplified gene locus, within the amplified HER2 gene, under conditions under which the TFOs enter the cancer cells, bind the polypurine site and induce DNA damage at the targeted amplified gene locus. In one embodiment, DNA damage is copy-number dependent DNA double strand breaks and DNA damage is sufficient to activate p53-independent apoptosis in cancer cells in which a HER2 gene is amplified. In some embodiments, the cancer cells in which a HER2 gene is amplified are HER2-amplified breast cancer cells or ovarian HER2-amplified breast cancer cells. The cancer cells are mammalian cells, generally human cells. The population of cells can be in an individual, such as in a human tissue, fluid or organ.

In some embodiments, the polypurine target site (also referred to as a targeted amplified gene locus) is/comprises SEQ ID NO: 1, SEQ ID NO: 2, SEQ ID NO: 5 or SEQ ID NO: 6. In some embodiments, the TFOs comprise at least one of the following: a nucleotide sequence identical to SEQ ID NO:3; a nucleotide sequence identical to SEQ ID NO:4; a nucleotide sequence identical to SEQ ID NO:7; a nucleotide sequence identical to SEQ ID NO:8; a nucleotide at least 90% identical to SEQ ID NO: 3; a nucleotide sequence at least 90% identical to SEQ ID NO: 4; a nucleotide sequence at least 90% identical to SEQ ID NO: 7 and a nucleotide at least 90% identical to SEQ ID NO: 8.

The polypurine site can be located in a variety of locations within an amplified cancer-promoting gene, such as in an amplified HER2 gene. For example, the polypurine site can be in the promoter region of the amplified cancer-promoting gene, such as in the promoter region of amplified HER2 gene; in a coding region of the amplified cancer-promoting gene, such as in the coding region of amplified HER2 gene; or in an intron of the amplified cancer-promoting gene, such as in an intron of amplified HER2 gene.

A further embodiment of the method is a method of activating apoptosis in cancer cells that comprise an amplified cancer-promoting gene, in a p53-independent manner. The method comprises contacting cancer cells comprising an amplified cancer-promoting gene with triplex forming oligonucleotides (TFOs) targeted to a polypurine site within the amplified cancer-promoting gene, under conditions under which the TFOs enter the cancer cells in sufficient quantity to induce copy number-dependent DNA damage and activate apoptosis. In the method, triplex-induced DNA damage is sufficient to activate p53-independent apoptosis in a copy number-dependent DNA damage manner. In the method, a population of cells comprising an amplified cancer-promoting gene is contacted with triplex forming oligonucleotides (TFOs) targeted to a polypurine target site (referred to as a targeted amplified gene locus) in the amplified cancer-promoting gene, under conditions under which the TFOs enter the cancer cells in sufficient quantity to induce apoptosis. DNA damage is, for example, DNA double strand breaks, such as copy number dependent DNA double strand breaks. In a specific example, the method is a method of inducing target-specific DNA damage in cancer cells in which a HER2 gene is amplified, the method comprising contacting (a) cancer cells in which a HER2 gene is amplified with (b) triplex forming oligonucleotides (TFOs) targeted to a polypurine site, referred to as a targeted amplified gene locus, within the amplified HER2 gene, under conditions under which the TFOs enter the cancer cells, bind the polypurine site and induce DNA damage at the targeted amplified gene locus. In one embodiment, DNA damage is copy-number dependent DNA double strand breaks and DNA damage is sufficient to activate p53-independent apoptosis in cancer cells in which a HER2 gene is amplified. In some embodiments, the cancer cells in which a HER2 gene is amplified are HER2-amplified breast cancer cells or ovarian HER2-amplified breast cancer cells. The cancer cells are mammalian cells, generally human cells. The population of cells can be in an individual, such as in a human tissue, fluid or organ.

In some embodiments, the polypurine target site (also referred to as a targeted amplified gene locus) is/comprises SEQ ID NO: 1, SEQ ID NO: 2, SEQ ID NO: 5 or SEQ ID NO: 6. In some embodiments, the TFOs comprise at least one of the following: a nucleotide sequence identical to SEQ ID NO:3; a nucleotide sequence identical to SEQ ID NO:4; a nucleotide sequence identical to SEQ ID NO:7; a nucleotide sequence identical to SEQ ID NO:8; a nucleotide at least 90% identical to SEQ ID NO: 3; a nucleotide sequence at least 90% identical to SEQ ID NO: 4; a nucleotide sequence at least 90% identical to SEQ ID NO: 7 and a nucleotide at least 90% identical to SEQ ID NO: 8.

The polypurine site can be located in a variety of locations within an amplified cancer-promoting gene, such as in an amplified HER2 gene. For example, the polypurine site can be in the promoter region of the amplified cancer-promoting gene, such as in the promoter region of amplified HER2 gene; in a coding region of the amplified cancer-promoting gene, such as in the coding region of amplified HER2 gene; or in an intron of the amplified cancer-promoting gene, such as in an intron of amplified HER2 gene.

One aspect of the present disclosure provides a method of reducing, in a population of cells, the number of cancer cells in which a gene is amplified. A specific embodiment of the method is a method of reducing, in a population of cells that comprises cancer cells, the number of cancer cells in which a HER2 gene is amplified, such as p53-depleted cancer cells in which a HER2 gene is amplified. The method comprises contacting such cancer cells with triplex forming oligonucleotides (TFOs) targeted to a polypurine target site in the amplified-HER2 gene, under conditions under which the TFOs enter the cancer cells in sufficient quantity to induce apoptosis. In some embodiments, the cancer cells in which a HER2 gene is amplified are p53-depleted cells mammalian cells. In some embodiments, the cancer cells in which a HER2 is amplified, such as p53-depleted cells in which a HER2 gene is amplified, are human cells. In some embodiments, the polypurine target site is/comprises SEQ ID NO: 1, SEQ ID NO: 2, SEQ ID NO:5 or SEQ ID NO:6. In some embodiments, the TFOs are at least 13 nucleotides in length. In some embodiments, the TFOs are at least 22 nucleotides in length. In some embodiments, at least 13 of the nucleotides hybridize to SEQ ID NO: 1, SEQ ID NO: 2, SEQ ID NO:5 or SEQ ID NO:6.

TFOs can be administered individually (e.g., all TFOs administered have the same sequence) or a combination of two or more TFOs can be administered (e.g., TFOs administered comprise different nucleotide sequences). In some embodiments, the TFOs comprise a nucleotide sequence at least 90% identical to SEQ ID NO: 3; a nucleotide sequence at least 90% identical to SEQ ID NO: 4; a nucleotide sequence at least 90% identical to SEQ ID NO: 7; a nucleotide at least 90% identical to SEQ ID NO: 8; or a combination of two, three or four of the foregoing. For example, TFOs administered can comprise a nucleotide sequence at least 90% identical to SEQ ID NO: 3 and a nucleotide sequence at least 90% identical to SEQ ID NO: 4; a nucleotide sequence at least 90% identical to SEQ ID NO: 3 and a nucleotide sequence at least 90% identical to SEQ ID NO: 7; a nucleotide sequence at least 90% identical to SEQ ID NO: 3 and a nucleotide at least 90% identical to SEQ ID NO: 8; a nucleotide sequence at least 90% identical to SEQ ID NO: 4 and a nucleotide sequence at least 90% identical to SEQ ID NO: 7; a nucleotide sequence at least 90% identical to SEQ ID NO: 4 and a nucleotide sequence at least 90% identical to SEQ ID NO:8; a nucleotide sequence at least 90% identical to SEQ ID NO: 7 and a nucleotide sequence at least 90% identical to SEQ ID NO: 8.

In some embodiments, three different TFOs are administered. For example, the following can be administered:
a TFO that comprises a nucleotide sequence at least 90% identical to SEQ ID NO: 3, a TFO that comprises a nucleotide sequence at least 90% identical to SEQ ID NO: 4 and a TFO that comprises a nucleotide sequence at least 90% identical to SEQ ID NO: 7;
a TFO that comprises a nucleotide sequence at least 90% identical to SEQ ID NO: 3, a TFO that comprises a nucleotide sequence at least 90% identical to SEQ ID NO: 4 and a TFO that comprises a nucleotide sequence at least 90% identical to SEQ ID NO: 8;
a TFO that comprises a nucleotide sequence at least 90% identical to SEQ ID NO: 3, a TFO that comprises a nucleotide sequence at least 90% identical to SEQ ID NO: 7 and a TFO that comprises a nucleotide sequence at least 90% identical to SEQ ID NO: 8; or
a TFO that comprises a nucleotide sequence at least 90% identical to SEQ ID NO: 4, a TFO that comprises a nucleotide sequence at least 90% identical to SEQ ID NO: 7 and a TFO that comprises a nucleotide sequence at least 90% identical to SEQ ID NO: 8.

In further embodiments, four different TFOs are administered: a (at least one) TFO that comprises a nucleotide sequence at least 90% identical to SEQ ID NO: 3; a (at least one) TFO that comprises a nucleotide sequence at least 90% identical to SEQ ID NO: 4; a (at least one) TFO that comprises a nucleotide sequence at least 90% identical to SEQ ID NO: 7; and a (at least one) TFO that comprises a nucleotide at least 90% identical to SEQ ID NO: 8.

As previously indicated, TFOs can be administered individually (e.g., all TFOs administered have the same sequence) or in combination, such as a combination of two or more TFOs (e.g., TFOs administered comprise different nucleotide sequences). In one embodiment, TFOs are administered individually and the individually administered TFOs are selected from: TFOs that comprise a nucleotide sequence identical to SEQ ID NO: 3; TFOs that comprise a nucleotide sequence identical to SEQ ID NO: 4; TFOs that comprise a nucleotide sequence identical to SEQ ID NO: 7; and TFOs that comprise a nucleotide sequence identical to SEQ ID NO: 8 (all TFOs administered have the same sequence, such as all TFOs administered comprise a nucleotide sequence identical to SEQ ID NO: 3; all TFOs administered comprise a nucleotide sequence identical to SEQ ID NO: 4; all TFOs administered comprise a nucleotide sequence identical to SEQ ID NO: 7; or all TFOs administered comprise a nucleotide sequence identical to SEQ ID NO: 8). In a further embodiment, a combination of two or more TFOs can be administered (e.g., TFOs administered comprise different nucleotide sequences).

For example, TFOs administered can comprise a nucleotide sequence identical to SEQ ID NO: 3 and a nucleotide sequence identical to SEQ ID NO: 4; a nucleotide sequence identical to SEQ ID NO: 3 and a nucleotide sequence identical to SEQ ID NO: 7; a nucleotide sequence identical to SEQ ID NO: 3 and a nucleotide sequence identical to SEQ ID NO: 8; a nucleotide sequence identical to SEQ ID NO: 4 and a nucleotide sequence identical to SEQ ID NO: 7; a nucleotide sequence identical to SEQ ID NO: 4 and a nucleotide sequence identical to SEQ ID NO:8; a nucleotide sequence identical to SEQ ID NO: 7 and a nucleotide sequence identical to SEQ ID NO: 8.

In some embodiments, three different TFOs are administered. For example, the following can be administered:
a TFO that comprises a nucleotide sequence identical to SEQ ID NO: 3, a TFO that comprises a nucleotide sequence identical to SEQ ID NO: 4 and a TFO that comprises a nucleotide sequence identical to SEQ ID NO: 7; or
a TFO that comprises a nucleotide sequence identical to SEQ ID NO: 3, a TFO that comprises a nucleotide sequence identical to SEQ ID NO: 4 and a TFO that comprises a nucleotide sequence identical to SEQ ID NO: 8; or
a TFO that comprises a nucleotide sequence identical to SEQ ID NO: 3, a TFO that comprises a nucleotide sequence identical to SEQ ID NO: 7 and a TFO that comprises a nucleotide sequence identical to SEQ ID NO: 8; or
a TFO that comprises a nucleotide sequence identical to SEQ ID NO: 4, a TFO that comprises a nucleotide sequence identical to SEQ ID NO: 7 and a TFO that comprises a nucleotide sequence identical to SEQ ID NO: 8.

In further embodiments, four different TFOs are administered: a (at least one) TFO that comprises a nucleotide sequence identical to SEQ ID NO: 3; a (at least one) TFO that comprises a nucleotide sequence identical to SEQ ID NO: 4; a (at least one) TFO that comprises a nucleotide sequence identical to SEQ ID NO: 7; and a (at least one) TFO that comprises a nucleotide identical to SEQ ID NO: 8.

In some embodiments, the triplex forming oligonucleotides (TFOs), such as TFOs identical to a TFO whose sequence is provided herein or at least 90% identical to a TFO whose sequence is provided herein, are in a delivery vehicle or are conjugated to a delivery vehicle. In some embodiments, the delivery vehicle is lipid nanoparticles. In some embodiments, the TFOs have backbone modifications. In some embodiments, the backbone modifications include phosphorothioates, phosphorodithioates, methylphosphonates, phosphoramidates, boranophosphate oligos, polyamides, methylene(methylimino) linkages, morpholino oligos, or some combination thereof. In some embodiments, the p53-depleted cancer cells are renal cell carcinoma cells, lung cancer cells, colon cancer cells, colon carcinoma cells, ovarian cancer cells, breast cancer cells, colorectal cancer cells, gastric cancer cells, and/or endometrial cancer cells.

Another aspect of the present disclosure provides a method of reducing, in a population of cells, the number of p53-mutated cancer cells in which a HER2 gene is amplified, the method comprising contacting p53-mutated cancer cells with triplex forming oligonucleotides (TFOs) targeted to a polypurine site in the amplified-HER2 gene, under conditions under which the TFOs enter the p53-mutated cancer cells in sufficient quantity to induce apoptosis. In some embodiments, the p53-mutated cells are mammalian cells. In some embodiments, the p53-mutated cells are human cells. In some embodiments, the polypurine target site is SEQ ID NO: 1, SEQ ID NO: 2, SEQ ID NO:5 or SEQ ID NO:6. In some embodiments, the TFOs are at least 13 nucleotides in length. In some embodiments, the TFOs are at least 22 nucleotides in length. In some embodiments, at least 13 of the nucleotides hybridize to SEQ ID NO: 1, SEQ ID NO: 2, SEQ ID NO:5 or SEQ ID NO:6. In some embodiments, the TFOs comprise a nucleotide sequence at least 90% identical to SEQ ID NO: 3, and/or a nucleotide sequence at least 90% identical to SEQ ID NO: 4, and/or a nucleotide sequence at least 90% identical to SEQ ID NO: 7 and/or a nucleotide at least 90% identical to SEQ ID NO: 8. In some embodiments, the TFOs are in a delivery vehicle or are conjugated to a delivery vehicle. In some embodiments, the delivery vehicle is lipid nanoparticles. In some embodiments, the TFOs have backbone modifications. In some embodiments, the backbone modifications include phosphorothioates, phosphorodithioates, methylphosphonates, phosphoramidates, boranophosphate oligos, polyamides, methylene(methylimino) linkages, morpholino oligos, or some combination thereof. In some embodiments, the p53-mutated cancer cells are renal cell carcinoma cells, lung cancer cells, colon cancer cells, colon carcinoma cells, ovarian cancer cells, breast cancer cells, colorectal cancer cells, gastric cancer cells, and/or endometrial cancer cells.

Another aspect of the present disclosure provides a method of treating cancer in an individual with Li-Fraumeni syndrome, the method comprising administering to the individual TFOs targeted to a polypurine target site in an amplified-HER2 gene, under conditions under which the TFOs enter p53-depleted cancer cells in sufficient quantity to induce apoptosis. In some embodiments, the polypurine target site is SEQ ID NO: 1, SEQ ID NO: 2, SEQ ID NO:5 or SEQ ID NO:6. In some embodiments, the TFOs are at least 13 nucleotides in length. In some embodiments, the TFOs are at least 22 nucleotides in length. In some embodiments, at least 13 of the nucleotides hybridize to SEQ ID NO: 1, SEQ ID NO: 2, SEQ ID NO:5 or SEQ ID NO:6. In some embodiments, the TFOs comprise a nucleotide sequence at least 90% identical to SEQ ID NO: 3, and/or a nucleotide sequence at least 90% identical to SEQ ID NO: 4, and/or a nucleotide sequence at least 90% identical to SEQ ID NO: 7 and/or a nucleotide at least 90% identical to SEQ ID NO: 8. In some embodiments, the TFOs are in a delivery vehicle or are conjugated to a delivery vehicle. In some embodiments, the delivery vehicle is lipid nanoparticles. In some embodiments, the TFOs are encapsulated in the lipid nanoparticles. In some embodiments, the TFOs have backbone modifications. In some embodiments, the backbone modifications include phosphorothioates, phosphorodithioates, methylphosphonates, phosphoramidates, boranophosphate oligos, polyamides, methylene (methylimino) linkages, morpholino oligos, or some combination thereof. In some embodiments, the TFOs are administered by injection. In some embodiments, the TFOs are administered intratumorally or intraperitoneally. In some embodiments, an anticancer agent that is not a TFO is administered with the TFOs. In some embodiments, the anticancer agent is a protein, a nucleic acid, a small molecule, or a drug. In some embodiments, the anticancer agent is a protein, a nucleic acid, a small molecule, or a drug.

Another aspect of the present disclosure provides a method of administering TFOs for the treatment of cancer, the method comprising preparing a mixture of TFOs targeted to a polypurine target site in an amplified-HER2 gene and administering the mixture of TFOs to an individual, in sufficient quantity to induce apoptosis. In some embodiments, the mixture of TFOs is encapsulated in lipid nanoparticles. In some embodiments, the polypurine target site is SEQ ID NO: 1, SEQ ID NO: 2, SEQ ID NO:5 or SEQ ID NO:6. In some embodiments, the TFOs are at least 13 nucleotides in length. In some embodiments, the TFOs are at least 22 nucleotides in length. In some embodiments, at least 13 of the nucleotides hybridize to SEQ ID NO: 1, SEQ ID NO: 2, SEQ ID NO:5 or SEQ ID NO:6. In some embodiments, the mixture of TFOs comprises a nucleotide sequence at least 90% identical to SEQ ID NO: 3, and/or a nucleotide sequence at least 90% identical to SEQ ID NO: 4, and/or a nucleotide sequence at least 90% identical to SEQ ID NO: 7 and/or a nucleotide at least 90% identical to SEQ ID NO: 8. In some embodiments, the TFOs have backbone modifications. In some embodiments, the backbone modifications include phosphorothioates, phosphorodithioates, methylphosphonates, phosphoramidates, boranophosphate oligos, polyamides, methylene(methylimino) linkages, morpholino oligos, or some combination thereof. In some embodiments, the mixture of TFOs is administered by injection. In some embodiments, the mixture of TFOs is administered intratumorally or intraperitoneally. In some embodiments, an anticancer agent that is not a TFO is administered with the mixture of TFOs. In some embodiments, the anticancer agent is a protein, a nucleic acid, a small molecule, or a drug. In some embodiments, the individual is a mammal. In some embodiments, the individual is a human. In some embodiments, the individual is a model of cancer. In some embodiments, the cancer is a carcinoma, a sarcoma or a melanoma with HER-2 gene amplification. In some embodiments, the model of cancer is selected from a group including a p53-knockout mouse, a Li-Fraumeni Syndrome mouse, a mouse with MDA-MB-453 cells, SKBR3 cells, BT474 cells, PEO1 cells, SKOV3 cells, and p53-knockout mouse.

Another aspect of the present disclosure provides a composition, comprising TFOs targeted to a polypurine target site in an amplified-HER2 gene in sufficient quantity to induce p53-independent apoptosis in a p53-depleted cancer cell or a p53-mutated cancer cell, a pharmaceutically acceptable carrier, and optionally lipid nanoparticles, wherein the TFOs are encapsulated in the lipid nanoparticles or are conjugated to the lipid nanoparticles. In some embodiments, the polypurine target site is SEQ ID NO: 1, SEQ ID NO: 2, SEQ ID NO:5 or SEQ ID NO:6. In some embodiments, the TFOs are at least 13 nucleotides in length. In some embodiments, the TFOs are at least 22 nucleotides in length. In some embodiments, at least 13 of the nucleotides hybridize to SEQ ID NO: 1, SEQ ID NO: 2, SEQ ID NO:5 or SEQ ID NO:6. In some embodiments, the TFOs comprise a nucleotide sequence at least 90% identical to SEQ ID NO: 3, and/or a nucleotide sequence at least 90% identical to SEQ ID NO: 4, and/or a nucleotide sequence at least 90% identical to SEQ ID NO: 7 and/or a nucleotide at least 90% identical to SEQ ID NO: 8. In some embodiments, the TFOs have backbone modifications. In some embodiments, the backbone modifications include phosphorothioates, phosphorodithioates, methylphosphonates, phosphoramidates, boranophosphate oligos, polyamides, methylene(methylimino) linkages, morpholino oligos, or some combination thereof. In some embodiments, the pharmaceutically acceptable carrier comprises water or saline.

Also described herein is a composition comprising: triplex forming oligonucleotides (TFOs) and poly (lactic acid)-hyperbranched polyglycerol (PLA-HPG) polymer nanoparticles. In one embodiment, the TFOs in the composition comprise at least one of the following: a nucleotide sequence identical to SEQ ID NO:3; a nucleotide sequence identical to SEQ ID NO:4; a nucleotide sequence identical to SEQ ID NO:7; a nucleotide sequence identical to SEQ ID NO:8; a nucleotide at least 90% identical to SEQ ID NO: 3; a nucleotide sequence at least 90% identical to SEQ ID NO: 4; a nucleotide sequence at least 90% identical to SEQ ID NO: 7 and a nucleotide at least 90% identical to SEQ ID NO: 8.

Another embodiment of the invention is a method of administering triplex forming oligonucleotides (TFOs) to an individual, comprising administering a composition comprising: triplex forming oligonucleotides (TFOs) and poly(lactic acid)-hyperbranched polyglycerol (PLA-HPG) polymer nanoparticles. In one embodiment, the TFOs in the composition comprise at least one of the following: a nucleotide sequence identical to SEQ ID NO:3; a nucleotide sequence identical to SEQ ID NO:4; a nucleotide sequence identical to SEQ ID NO:7; a nucleotide sequence identical to SEQ ID NO:8; a nucleotide at least 90% identical to SEQ ID NO: 3; a nucleotide sequence at least 90% identical to SEQ ID NO: 4; a nucleotide sequence at least 90% identical to SEQ ID NO: 7 and a nucleotide at least 90% identical to SEQ ID NO: 8 to an individual by intravenous injection.

The compositions and methods disclosed herein constitute a new paradigm for targeted therapeutics, efficacious in the treatment of cancers driven by gene amplification with minimal potential for toxicity to normal tissue. Also described herein are agents with a unique mechanism of action and results that show that induction of the DNA damage response via TFO treatment is as effective as targeting the overexpressed oncogenic protein product. HER2-205 treatment of HER2-positive breast cancer xenografts resulted in a 52% reduction in tumor volumes compared to controls, which is comparable to the 58% reduction observed with trastuzumab. Enhanced tumor delivery (delivery to a tumor) using a NP platform can significantly improve the efficacy of TFO-treatment. Furthermore, development of bioactive reagents is not restricted to polypurine sequences within a specific region of the amplified gene. The compositions and methods disclosed herein can be used as a drug design platform and treatment option for several cancers with gene amplification and resistance to current therapies.

The details of one or more embodiments of the invention are set forth in the description below. Other features or advantages of the present invention will be apparent from the drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure. In the drawings:

FIGS. 1A-1E include diagrams showing targeting of gene amplification in cancer via triplex formation. FIG. 1A: a schematic illustration showing a drug design scheme. Targeting the HER2 gene on a genomic level using DNA-binding molecules provides a novel therapeutic option to directly manipulate the DNA damage response pathways to specifically attack the HER2-amplified tumor. Triplex-induced DNA damage will only provoke apoptosis when multiple triplex structures are formed, while nucleotide excision repair (NER)-dependent repair prevails in the presence of one or two structures. FIG. 1B: a table showing the gene copy number characteristics of breast cancer cells lines¹⁸. FIG. 1C: a photo showing the Western blot analysis of HER2 protein levels in breast cancer cell lines with varying gene copy number. FIG. 1D: a schematic illustration showing that TFOs bind as third strands in a sequence-specific manner within the major groove of duplex DNA at polypurine sites. The specificity of these molecules arises from the formation of base triplets via reverse Hoogsteen hydrogen bonds between the third strand and the polypurine strand of the duplex DNA. Results shown are from the use of TFOs HER2-1 and HER2-205, designed to bind to a polypurine sequence located either in the promoter or the coding region of the HER2 gene. FIG. 1E: photos of non-denatured metaphase chromosome spreads of MCF7 and BT474 breast cancer cells demonstrate chromosomal binding of TAMRA-HER2-205 (red) to its target site located on chromosome (chr.) 17 (green).

FIGS. 2A-2H include diagrams showing that triplex induced DNA damage and apoptosis correlate with gene copy number. “UT”=untreated cells. “Mock”=cells with transfection reagent only. “MIX24”=cells treated with control mixed sequence oligonucleotide, MIX24. “HER2-1”=HER2-1-treated cells. “HER2-205”=HER2-205-treated cells. FIG. 2A: a chart showing characterization of TFO induced DNA damage in BT474 cells as measured by neutral comet assay 24 h post-treatment. FIG. 2B: a chart showing the quantification of triplex-induced DNA double strand breaks using the neutral comet assay as measured by tail moment in multiple breast cancer cell lines. FIG. 2C: a chart showing that triplex-induced DNA damage increases in cell lines containing multiple copies of the HER2 gene. FIG. 2D: a chart showing the frequency of cells with more than 5 γH2AX foci per nuclei following 24 h HER2-205 treatment. FIG. 2E: representative images of HER2-205 induced 53BP1 (green) and γH2AX (red) foci in nuclei (blue) compared to MIX24 24 h post-treatment in BT474 cells. FIG. 2F: a chart showing the analysis of triplex-induced apoptosis as measured by Annexin-V staining in breast cancer cell lines 24 h post TFO-treatment. FIG. 2G: a chart showing that the level of triplex-induced apoptosis increases with gene copy number. FIG. 2H: a chart showing the analysis of triplex-induced apoptosis in HER2-positive ovarian cancer cells as measured by Annexin V staining 48 h post-treatment. The TFO concentration used in these studies is 2 μg/well (˜100 nM). **** denotes p<0.0001, *** denotes p<0.001, ** denotes p<0.01, and * denotes p<0.05.

FIGS. 3A-3J include diagrams showing the in vivo effect of HER2-205 on human HER2-positive cancer xenografts. FIG. 3A: Ex vivo fluorescence imaging of TAMRA-HER2-205 uptake in tumors harvested 6 h and 12 h post IP injection. FIG. 3B: Confocal microscopy imaging of tumor tissue from mice treated with TAMRA-HER2-205 (20 mg/kg). Representative images of tumors sections at 6 h and 12 h post dosing. FIG. 3C: Tumor sections visualize DAPI (blue), phalloidin (green) and HER2-205 (red) following a single dose of TAMRA-HER2-205 (20 mg/kg). Tumor growth delay curves of BT474 xenografts generated by subcutaneous injection of female athymic nude mice. Twenty-eight days after implantation mice were treated by intraperitoneal (IP) injection with three 20 mg/kg doses (evenly administered over 7 days) of HER2-205, n=5 (FIG. 3D), trastuzumab, n=8 (FIG. 3E) and MIX24, n=8 (FIG. 3F), at a concentration of 20 mg/kg. Arrow indicates administration of doses. Tumor growth measurements ±SEM are shown. FIG. 3G: a Kaplan-Meier plot of the percentage of tumors smaller than three times baseline size. Baseline size was defined as tumor size on the first day of treatment [Day 28 in FIG. 3D and Day 21 in FIG. 3E and FIG. 3F]. FIG. 3H: images showing a histopathologic analysis of BT474 tumor sections from mice 24 h after treatment with a single dose of HER2-205 (20 mg/kg body weight) or vehicle. Haematoxylin and eosin (H&E), caspase 3, HER2, Ki67 stain at 4× magnification. Scale bar=10 μm. FIG. 3I: an image showing a higher magnification of H&E tumor section from HER2-205 treatment specimen. FIG. 3J: a Kaplan-Meier plot of the percentage of SKOV3 ovarian cancer tumors smaller than three times baseline size. Mice were treated with 3 doses of HER2-205 (n=5) at a concentration of 20 mg/kg or cisplatin (n=7) at a concentration of 10 mg/kg.

FIGS. 4A-4D includes diagrams showing triplex targeting of non-essential regions of the HER2 gene. FIG. 4A: a diagram showing TFOs were designed to bind to polypurine sequences located in the introns of the HER2 gene. HER2-5922 was designed to bind to a region of intron 2 and HER2-40118 targets a site in intron 19. FIG. 4B a chart showing characterization of TFO induced DNA damage in BT474 cells as measured by neutral comet assay 24 h post-treatment. FIG. 4C a chart showing evaluation of dose-dependent cell viability using CellTiter-Glo luminescent assay 48 h post-treatment with HER2-targeted TFOs in BT474 cells. FIG. 4D: images showing Western blot analysis of triplex-induced apoptosis as measured by cleaved PARP and DNA damage by pH2AX S139 in BT474 cells 12 h following TFO-treatment. **** denotes p<0.0001, *** denotes p<0.001, and ** denotes p<0.01.

FIGS. 5A-5H include diagrams showing the molecular mechanism of anticancer activity. “UT”=untreated cells. “Mock”=cells with transfection reagent only. “MIX24”=cells treated with control mixed sequence oligonucleotide, MIX24. “HER2-205”=HER2-205-treated cells. FIG. 5A: a chart showing that CUP analysis of BT474 cells demonstrate gene-specific enrichment of γH2AX at the HER2 target site and an absence of DNA damage at the non-targeted GAPDH locus 8 h post-treatment with HER2-205. FIG. 5B: images showing a Western blot analysis of the phosphorylation status of the DNA damage response proteins Chk1 and Chk2 following TFO treatment. FIG. 5C: images showing that the knockdown of the NER factor, XPD, in BT474 cells results in a decrease in the induction of apoptosis as measured by cleaved PARP and pH2AX Y142. pH2AX Y142 is an essential post-translational modification for the recruitment of pro-apoptotic factors to the tail of γH2AX. FIG. 5D: images showing that HER2-205 activates p53-independent apoptosis in HER2-positive BT474 cells. FIG. 5E: a chart showing the analysis of HER2 gene expression by RT-PCR and FIG. 5F: images showing that determination of HER2 protein levels and phosphorylation status using Western blot analysis provide evidence that HER2-205 achieves therapeutic activity using a mechanism that is independent of HER2 cellular function. FIG. 5G: images showing that inhibition of transcription in BT474 cells prior to TFO treatment results in a similar level of triplex-induced DNA damage and apoptosis as indicated by Western blot analysis of pH2AX S139 and cleaved caspase 3. FIG. 5H: a schematic illustration of molecular mechanism of gene-targeted apoptosis. TFO binding in the major groove of duplex DNA causes a distortion of the double helix, which can induce DNA replication fork collapse and induction of DNA double strand breaks (DSBs). DNA damage response activates an XPD-dependent but p53-independent apoptotic pathway.

FIGS. 6A-6G include diagrams showing the impact of nanoparticle delivery on therapeutic efficacy. “UT”=untreated cells. “Mock”=cells with transfection reagent only. “MIX24”=cells treated with control mixed sequence oligonucleotide, MIX24. “HER2-205”=HER2-205-treated cells. “HER2-5922”=HER2-5922-treated cells. FIG. 6A: confocal imaging of tumor tissue from mice treated with TAMRA-HER2-205 encapsulated PLA-HPG nanoparticles (NPs) visualize tumor distribution. Representative images of tumor sections 12 and 24 hours post intravenous administration via retro-orbital injection. FIG. 6B: charts showing that intratumor TAMRA fluorescence was detected and quantified at both 12 and 24 hours after a 2 mg dose of TAMRA-HER2-205 PLA-HPG NPs (n=4 tumors/timepoint). TFO uptake in the total tumor and the sub-compartments of nuclear and extranuclear was quantified as mean fluorescence intensity (MFI). Statistical significance was calculated by ordinary one-way ANOVA and Kolmogorov-Smirnov test. FIG. 6C: a chart showing tumor growth delay curves in an orthotopic breast cancer model generated from BT474 cells in female athymic nude mice. Twenty-four days after implantation mice were treated by systemic administration with a 2 mg dose of PLA-HPG encapsulated NPs of HER2-205, n=8; HER2-5922, n=8 and MIX24, n=8. Arrow indicates administration of the first dose of seven evenly administered over 15 days. Tumor volume measurements ±SEM are shown. FIG. 6D: a Kaplan-Meier plot of the percentage of tumors smaller than three times baseline size. Baseline size was defined as tumor size on the first day of treatment. FIG. 6E: a chart showing body weight monitored as a means to detect gross toxicity. FIG. 6F: images showing that immunofluorescence of γH2AX indicate an increase in DNA damage in tumors following treatment with HER2-205 PLA-HPG NPs (n=4). Representative images of confocal microscopy 12 h post-treatment and quantification of γH2AX foci is reported as mean fluorescence intensity. FIG. 6G: representative images of confocal microscopy of cleave caspase 3 immunofluorescence in tumors 24 h post HER2-205 PLA-HPG NP treatment (n=4). Quantification of mean fluorescence intensity indicates an activation of apoptosis in tumors.

FIGS. 7A-7J “UT”=untreated cells. “Mock”=cells with transfection reagent only. “MIX24”=cells treated with control mixed sequence oligonucleotide, MIX24. “HER2-1”=HER2-1-treated cells. “HER2-205”=HER2-205-treated cells. FIG. 7A: representative images of neutral comet assays performed 24 h after HER2-205 treatment in MCF7 and BT474 cells. FIG. 7B: a chart showing the quantification of cells with greater than 5 γH2AX and/or 53BP1 foci per nuclei in BT474 cells treated with HER2-205 or MIX24. FIG. 7C: images showing that triplex formation induces apoptosis in HER2-positive breast cancer cell lines as measured by Western blot analysis of cleaved PARP. FIG. 7D: images showing the detection of HER2 copies in interphase nuclei by dual color FISH with HER2 probe (red) and chromosome 17 probe (green). FIG. 7E: images showing the immunofluorescence of γH2AX in PEO1 ovarian cancer cells 24 h post-treatment with HER2-205 or MIX24. FIG. 7F: representative immunofluorescence images of γH2AX foci in SKOV3 ovarian cancer cells 24 h following treatment with HER2-205 or MIX24. FIG. 7G: a chart showing the frequency of PEO1 and SKOV3 cells positive for γH2AX following 24 h treatment. FIG. 7H: a chart showing the quantification of triplex-induced DNA double strand breaks using the neutral comet assay as measured by tail moment. FIG. 7I: images showing a monolayer growth assay that demonstrates a decrease in cell survival in PEO1 and SKOV3 cells treated with HER2-205 72 h after treatment. FIG. 7J: images showing a Western blot analysis of activation of apoptosis as measured by cleaved PARP in ovarian cancer cells following TFO treatment.

FIGS. 8A-8J “UT”=untreated cells. “Mock”=cells with transfection reagent only. “MIX24”=cells treated with control mixed sequence oligonucleotide, MIX24. “HER2-205”=HER2-205-treated cells. FIG. 8A: a chart showing ChIP analysis of γH2AX in BT474 cells detected increased DNA damage at the targeted HER2 gene following HER2-5922 treatment. FIG. 8B: a chart showing the quantification of phosphorylated ATM by flow cytometry following treatment with HER2-205. FIG. 8C: a chart showing analysis of HER2 gene expression by RT-PCR 12 h post-treatment with HER2-targeted TFOs. FIG. 8D: a chart showing quantification of triplex-induced DNA double strand breaks using the neutral comet assay as measured by tail moment 12 h post TFO treatment. FIG. 8E: images showing Western blot analysis of activation of apoptosis as measured by cleaved PARP and pH2AX Y142 12 h following TFO treatment. FIGS. 8F-8H: images showing Western blot analysis of the phosphorylation status of HER family receptors HER3 (FIG. 8F), HER4 (FIG. 8G), and EGFR (HER1) (FIG. 8H) in multiple breast cancer cell lines following HER2-205 treatment. FIG. 8I: a chart showing analysis of HER2 gene expression by RT-PCR 12 h post-treatment with HER2-targeted TFOs. FIG. 8J: a chart showing analysis of HER2 gene expression by RT-PCR 20 h following pretreatment with the transcription inhibitor, α-amanitin.

FIGS. 9A-9B include diagrams showing a comparison of PLGA and PLA-HPG NPs in vivo. FIG. 9A: images showing uptake of DiD-loaded NPs, PLGA/DCM, PLGA/EtOAc and PLA-HPG, 12 h after systemic administration via retro-orbital injection. Tumor cryosections visualize DAPI (blue) and DiD (red). FIG. 9B: images showing biodistribution of DiD-loaded PLA-HPG NPs 12 h after systemic administration. DiD fluorescence in isolated organs after retro-orbital injection with DiD encapsulated NPs (2 mg).

FIGS. 10A-10E show biodistribution of TAMRA-HER2-205 encapsulated PLA-HPG nanoparticles (NPs). “UT”=untreated cells. “HER2-205”=HER2-205-treated cells. FIG. 10A shows representative confocal images of tissue sections 12 hours post intravenous administration via retro-orbital injection of a 2 mg dose of NPs. FIG. 10B shows representative confocal images of TAMRA-HER2-205 biodistribution in tissues 24 hours post treatment. FIG. 10C includes charts showing that TAMRA fluorescence was quantified at both 12 and 24 hours after dosing (2 mg of NPs) and TFO uptake in each tissue is reported as mean fluorescence intensity (MFI). Statistical significance was calculated by ordinary one-way ANOVA and Kolmogorov-Smirnov test. FIG. 10D is a pie chart showing analysis of TAMRA-HER2-205 biodistribution 12 hours post treatment. Fluorescence intensity observed in each tissue is reported as a percentage of the combined total fluorescence intensity detected in spleen, kidney, liver and tumor (tumor data is shown and quantified in FIGS. 6A, 6B. Total area of the pie chart denotes the sum of the absolute fluorescence within the four organs, representing the total TFO uptake by these organs, and each slice gives the relative HER2-205 uptake for each organ. FIG. 10E is a pie chart showing analysis of TAMRA-HER2-205 biodistribution 24 hours post systemic administration. Fluorescence intensity observed in each tissue is reported as a percentage of the combined total fluorescence intensity detected in spleen, kidney, liver and tumor (tumor data is shown and quantified in FIGS. 6A, 6B. Total area of the pie chart denotes the sum of the absolute fluorescence within the four organs, representing the total TFO uptake by these organs, and each slice gives the relative HER2-205 uptake for each organ.

FIGS. 11A-11E. “UT”=untreated cells. “Mock”=cells with transfection reagent only. “MIX24”=cells treated with control mixed sequence oligonucleotide, MIX24. “HER2-205”=HER2-205-treated cells. “HER2-5922”=HER2-5922-treated cells. FIG. 11A: charts showing nanoparticle characterization. Nanoparticle diameter as measured by dynamic light scattering (DLS). Nanoparticle surface charge measured by zeta potential. Nanoparticle loading of TFOs measured by extraction and analysis. All data is plotted as mean±SEM, n=3. FIG. 11B: representative images of confocal microscopy of γH2AX immunofluorescence in tumors 24 h post-treatment with HER2-205 PLA-HPG NPs and quantification of γH2AX foci is reported as mean fluorescence intensity. FIG. 11C: representative images of confocal microscopy of cleaved caspase 3 immunofluorescence in tumors 12 h post-treatment with HER2-205 PLA-HPG NPs and quantification of activated caspase 3 is reported as mean fluorescence intensity. FIG. 11D: images showing HER2 immunofluorescence analysis of BT474 tumor sections from mice 12 h and 24 h after treatment with a single dose of HER2-205 PLA-HPG NPs (2 mg). FIG. 11E: confocal microscopy images of tumor sections analyzed by immunofluorescence 12 h and 24 h following a single dose of TAMRA-HER2-205 PLA-HPG NPs.

FIGS. 12A-12D. FIG. 12A is an analytical ESI-MS spectrum of HER2-205. FIG. 12B is an analytical reverse-phased HPLC of HER2-205. FIG. 12C is an analytical ESI-MS spectrum of HER2-5922. FIG. 12D is an analytical reverse-phased HPLC of HER2-5922.

SEQUENCES PRESENTED

5′-AGGAGAAGGAGGAGGTGGAGGAGGAGGG-3′
(SEQ ID NO: 1)
3′-CCCCGAGGAGGAGCGGGAGAACGGGGGG-5′
(SEQ ID NO: 2)
5′-GGGAGGAGGAGGTGGAGGAGGAAGAGGA-3′;
SEQ ID NO: 3)
5′-GAGGAGGAGTGGGAGAATGGGGGG-3′;
SEQ ID NO: 4)
3′-GGGAAAGAGGAGGGGGTGAGAGGAGTGGGG -5′
(SEQ ID NO: 5)
3′-GGGGGAAACAGGGAGGGTGGGG-5′
(SEQ ID NO: 6)
5′-GGGAAAGAGGAGGGGGTGAGAGGAGTGGGG-3′
(SEQ ID NO: 7)
5′-GGGGGAAATAGGGAGGGTGGGG-3′
(SEQ ID NO: 8)
5′-AGTCAGTCAGTCAGTCAGTCAGTC-3′
(SEQ ID NO: 9)
5′-CCCTCCTCCTCCACCTCCTCCTTCTCCT-3′
(SEQ ID NO: 10)
5′-GGGGCTCCTCCTCGCCCTCTTGCCCCCC-3′
(SEQ ID NO: 11)
5′-CCCTTTCTCCTCCCCCACTCTCCTCACCCC-3′
(SEQ ID NO: 12)
5′-CCCCCTTTGTCCCTCCCACCCC-3′
(SEQ ID NO: 13)
5′ GAC AGT CGA GAC GCT CAG G -3′
(SEQ ID NO: 14)
5′ GGA AAG CGC CAG TCT CTT GG -3′
(SEQ ID NO: 15)
5′ GCC TTGTAG CTA AGG ATC ACC- 3′
(SEQ ID NO: 16)
5′ CAA CCC CCA GGA CGA AAA AAG- 3′
(SEQ ID NO: 17)

### DETAILED DESCRIPTION

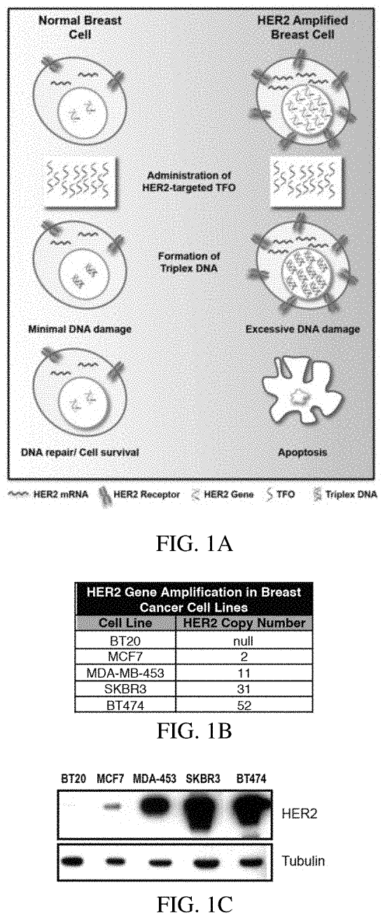

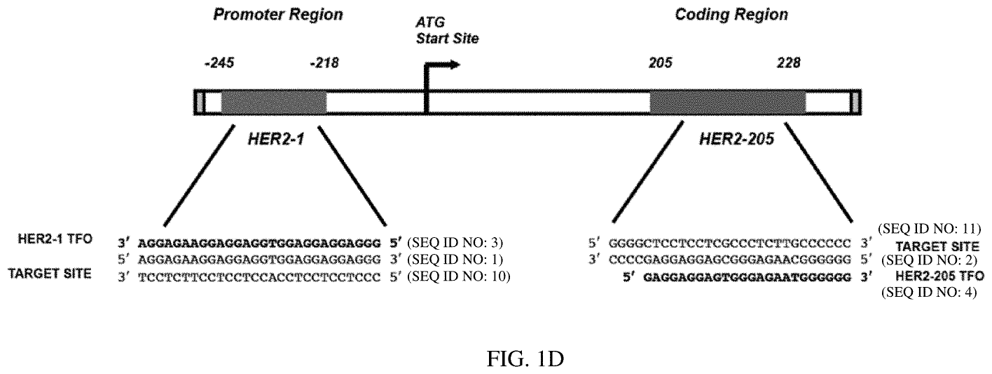

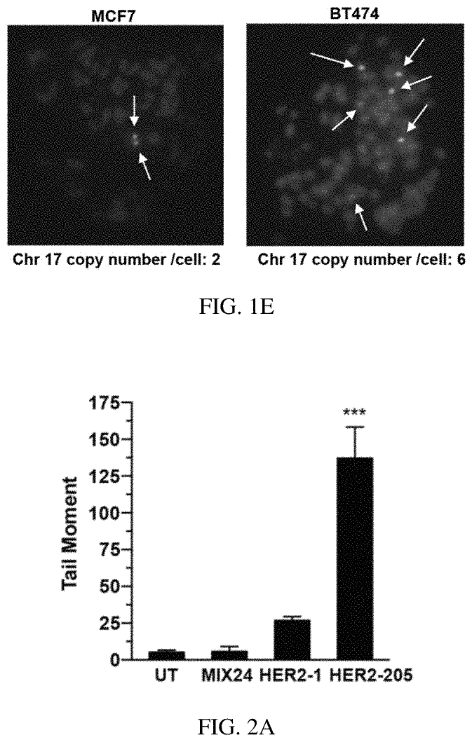

The present disclosure relates to a drug platform that converts (e.g., directly converts) amplified genes such as amplified oncogenic driver genes into DNA damage to trigger cell death (FIG. 1A). This approach employs TFOs that recognize unique polypurine sites within an amplified chromosomal region¹⁴⁻¹⁶. Binding of TFOs within the major groove of the double helix causes DNA perturbation and double strand break (DSB) formation; for example, DNA perturbation can impede replication fork progression, resulting in fork collapse and DSB formation¹⁷. Formation of multiple chromosomal triplex structures induces sufficient DNA damage to activate apoptosis in human cells¹⁸. The nucleotide excision repair (NER) pathway resolves low levels of triplex-induced DNA damage and hence normal cells can tolerate TFO treatment^{19,20}.

Amplified regions of a gene can span kilobases to tens of megabases that include multiple oncogenic genes, as well as passenger genes. Across the 14 human cancer subtypes characterized by gene amplification, the majority should have amplified regions with sequences that are conducive for a TFO approach, such as the present TFO approach.

For example, HER2 gene amplification in breast cancers provides an opportunity to test the efficacy of TFOs as a specific apoptosis-inducing agent in cancer cells, with limited toxicity in healthy cells, which lack HER2 amplification²¹ (FIGS. 1A-1C). This approach is particularly feasible due to the presence of several polypurine sites in the HER2 gene that are a prime target for triplex formation¹⁵.

A TFO, HER2-1, was designed to target the polypurine sequence in the promoter region of the HER2 gene at positions −218 to −245, relative to the transcription start site (FIG. 1D). Another polypurine site favorable for high affinity triplex formation is located within the coding region beginning at position 205 and is targeted by TFO, HER2-205 (FIG. 1D). To confirm chromosomal TFO binding, non-denatured metaphase spreads were prepared from MCF7 and BT474 breast cancer cells that had been treated with TAMRA-labeled HER2-205. The generation of chromosomal HER2-205 foci represent third strand binding to fixed chromosomes with intact DNA double helix, indicative of triplex formation¹⁸. Using a FITC labeled satellite probe specific for human chromosome 17, gene-specific triplex formation was verified (FIG. 1E). TAMRA-HER2-205 chromosomal foci were only generated on chromosome 17, the location of the HER2 gene, thus validating target site specificity (FIG. 1E).

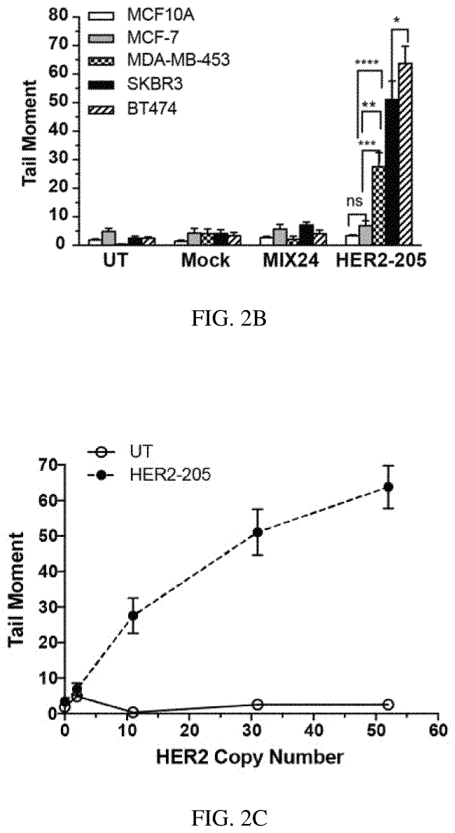

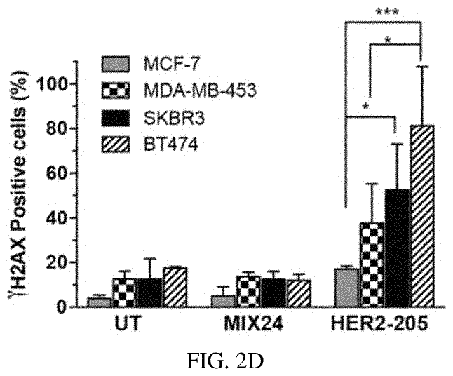

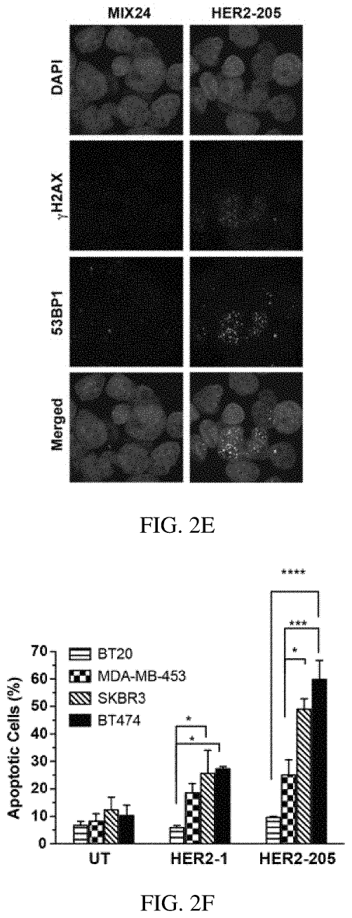

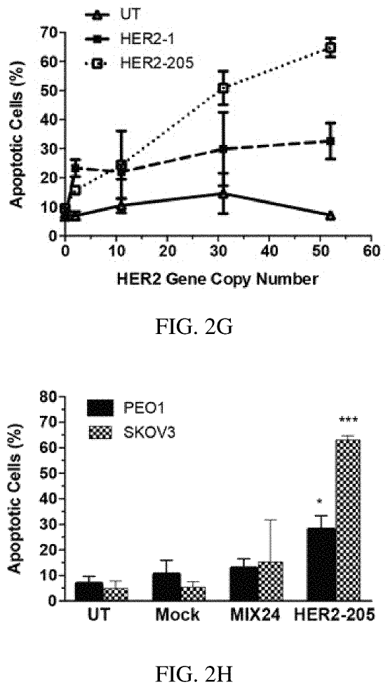

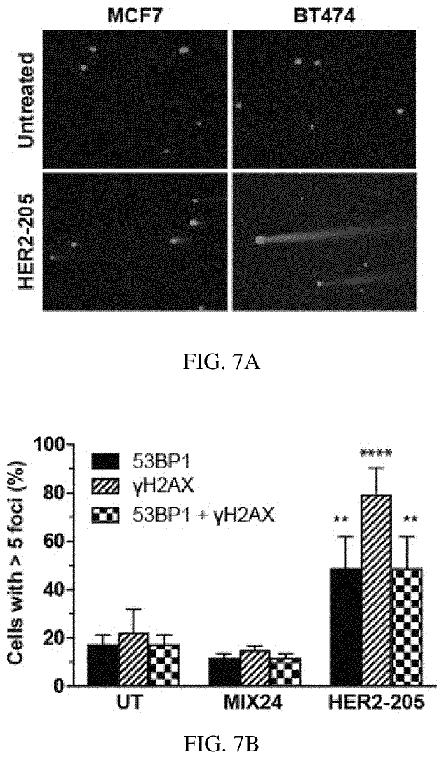

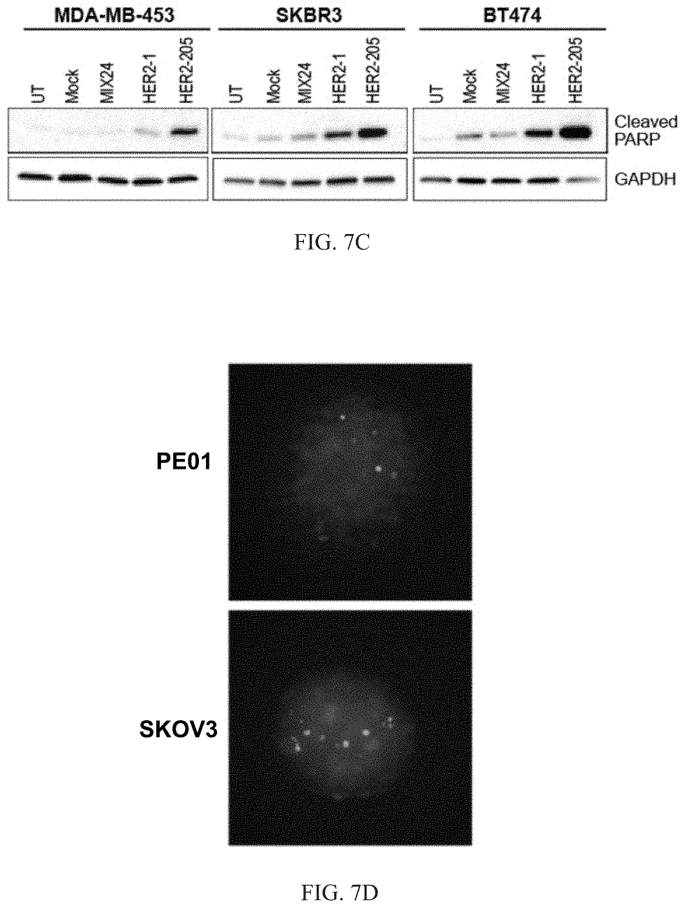

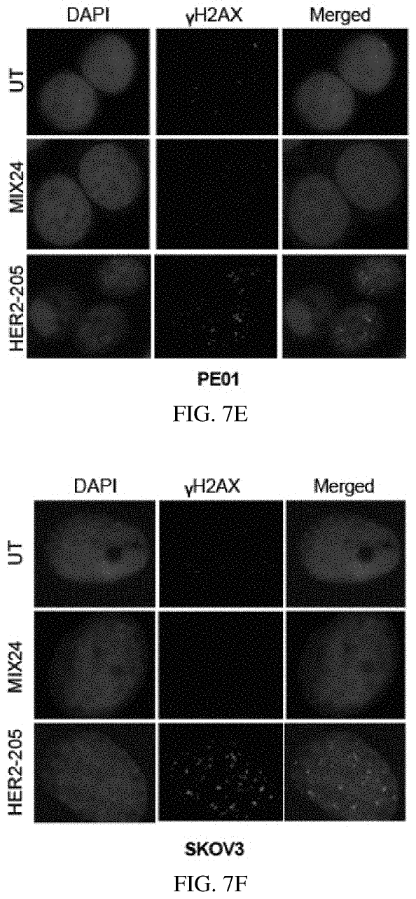

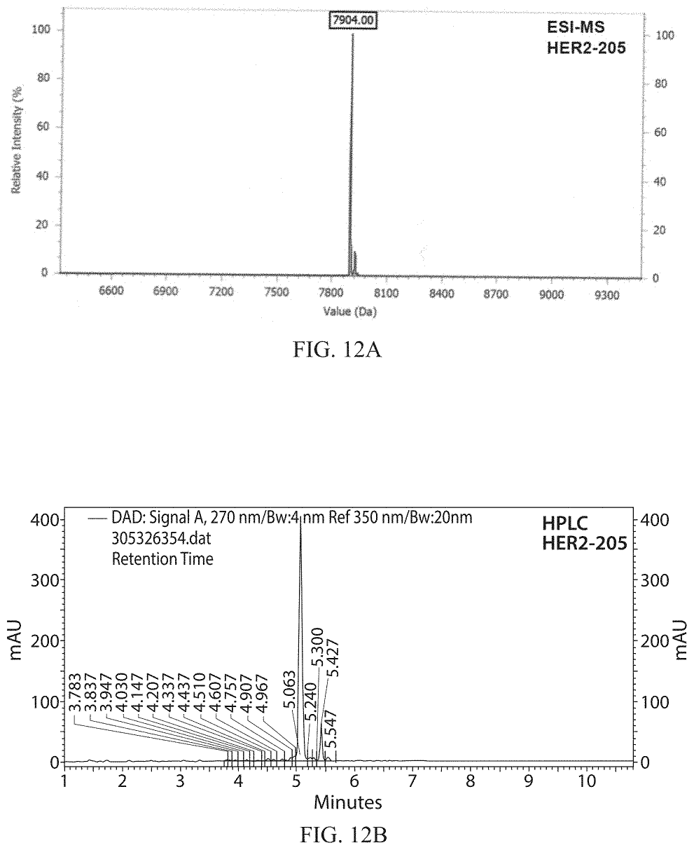

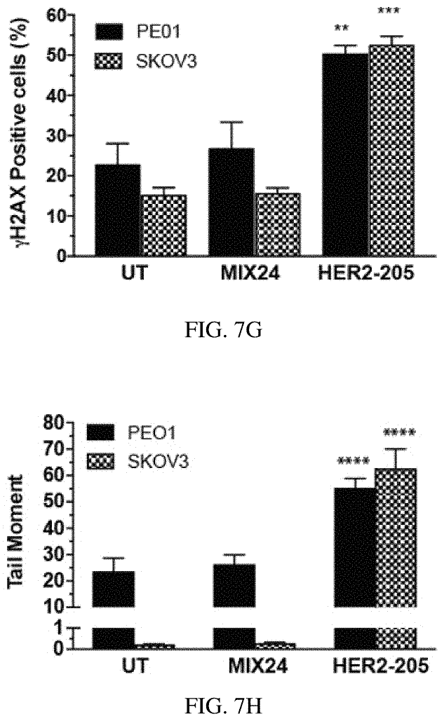

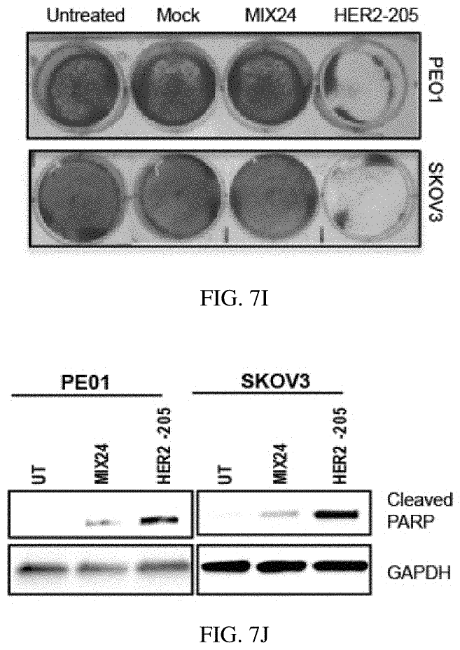

Next, it was assessed whether the level of triplex-induced DNA damage correlated with higher gene copy numbers. A neutral comet assay was used to establish that HER2-205 was more effective at inducing DNA damage than HER2-1, as indicated by an increase in DNA tail moment (FIG. 2A). Additionally, HER2-205 induced significantly more DSBs in cell lines containing multiple copies of the HER2 gene (FIGS. 2B and 7A). Results showed that the level of triplex-induced DNA damage was directly proportional to gene copy number (FIG. 2C). Markedly increased γH2AX positive cells (also referred to as gammaH2AX or gH2AX), indicative of DSBs, were observed upon treatment of breast cancer cells with high HER2 gene copy numbers (FIG. 2D). 53BP1 foci was also assessed, which colocalizes with γH2AX at damage sites. HER2-205 treated BT474 cells exhibited substantially increased γH2AX and 53BP1 foci, compared to cells treated with the control oligonucleotide MIX24 (FIG. 2E). Furthermore, colocalization of γH2AX and 53BP1 was observed in 49% of cells following HER2-205 treatment (FIG. 7B).

Given the association of increased DNA damage with activation of apoptosis, it was hypothesized that HER2-targeting TFOs would be capable of inducing apoptosis specifically in amplified breast cancer cells. Assessment of effects of Her2-targeting TFOs demonstrated TFO-induced apoptosis specifically in the HER2-positive cell lines and that HER2-205 treatment resulted in a higher percentage of apoptotic cells than treatment with HER2-1 (FIGS. 2F, 2G, and 7C). Together, the results demonstrate that the intensity of triplex-induced DNA damage and apoptosis is dependent on—related to/correlated with—gene copy number (FIGS. 2C, 2G). Furthermore, these results show that triplex-induced apoptosis provides the basis for novel therapeutics that specifically target cancers stemming from gene amplification, while sparing normal non-amplified tissues.

To demonstrate the adaptability of this technology to target other cancers, therapeutic efficacy was evaluated in HER2-positive ovarian cancers. When administered to PEO1 and SKOV3 cells, both of which have HER2 copy number gains (FIG. 7D), HER2-205 treatment induced increased γH2AX foci and DNA tail moments (FIGS. 7E-7H). Elevated levels of unrepaired DSBs were also observed in the untreated PEO1 cells, which harbor a deficiency in BRCA2, a key factor involved in DSB repair by homologous recombination (FIGS. 7G, 7H). Importantly, TFO treatment significantly increased the level of DSBs above baseline (FIG. 7H). In addition, HER2-205 reduced cell viability (FIG. 7I) and activated apoptosis in both cancer cell lines (FIGS. 2H and 7J).

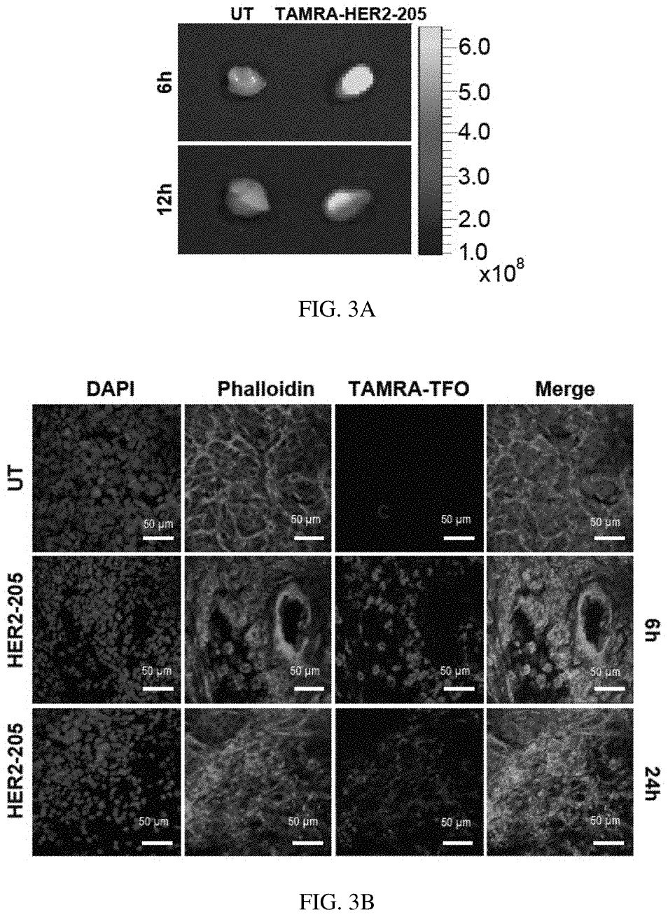

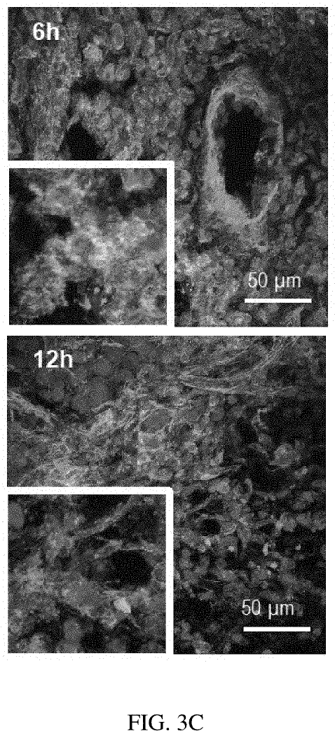

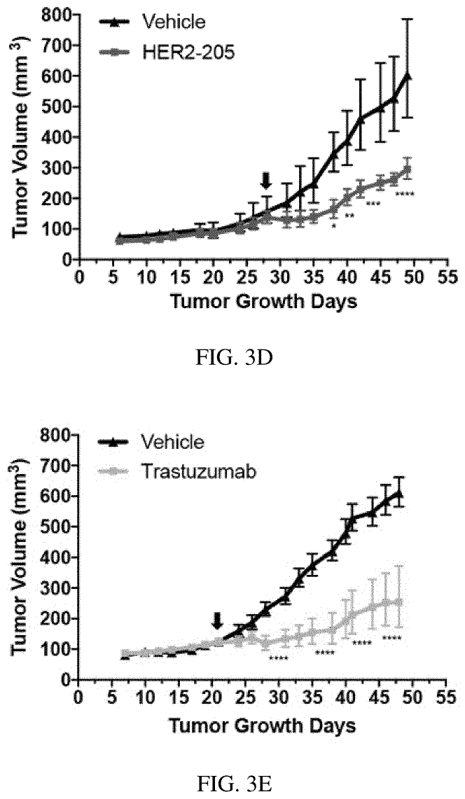

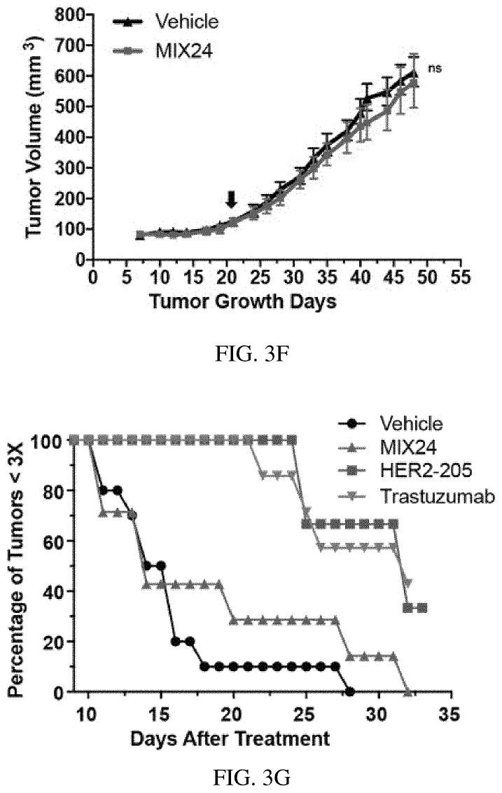

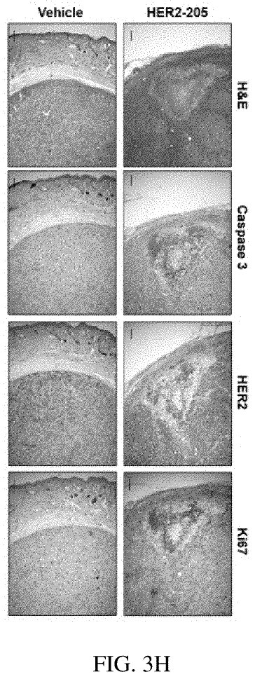

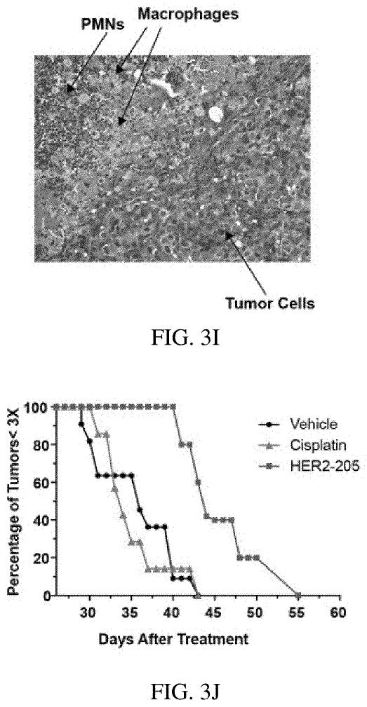

It was reasoned that the HER2-targeting TFO could have clinical efficacy in treating HER2-positive cancers. Two independent subcutaneous xenograft tumor models were developed to test this premise and confirmed TFO tumor uptake by ex vivo fluorescence imaging and confocal microscopy of tumor tissue using TAMRA-labeled HER2-205 (FIGS. 3A-3C). Importantly, treatment of BT474 human breast cancer tumors in athymic nude mice with HER2-205 suppressed tumor growth to a significantly greater degree than in the control group of animals treated with MIX24 (FIGS. 3D, 3F). IP administration of HER2-205 resulted in a notable reduction in tumor growth that was comparable to the currently used targeted therapy trastuzumab, thus demonstrating the utility of this gene-targeted cancer therapy (FIG. 3D, 3E). A tumor tripling time of 29±5.7 days post-initial dose was observed in tumors treated with HER2-205, compared to 24±2.1 days in tumors treated with trastuzumab (FIG. 3G). In contrast, the control oligonucleotide, MIX24 had no impact on BT474 tumor growth relative to the control buffer alone, with a tumor tripling time for control tumors of 15.7±4.9 days versus 16.3±6.6 days in tumors treated with MIX24 (ANOVA, p=0.99; FIG. 3G). Histological and immunohistochemical analyses were performed on paraffin-embedded tumor tissue sections. Tumor cell apoptosis (evidenced by the presence of cleaved caspase 3), decreased proliferation, as measured by Ki67 staining and a confluent area of tumor necrosis were observed in the HER2-205 treated specimen (FIG. 3H). Magnification of the HER2-205 treated tumor revealed that areas of tumor cell apoptosis are accompanied by a brisk infiltrate of inflammatory cells consisting predominantly of neutrophils and macrophages (FIG. 3I).

The standard of care for epithelial ovarian cancers consists of platinum-based chemotherapy and surgical cytoreduction²². However, as in the case of the SKOV3 cell line, many human ovarian cancers are resistant to platinum-based drugs. Using SKOV3 ovarian cancer xenografts, it was found that HER2-205 treatment confers a substantial survival advantage compared with cisplatin (FIG. 3J). HER2-205 demonstrated significant tumor growth inhibitory activity; the average tumor volume was 49% smaller than that of tumors in cisplatin-treated mice (ANOVA, p=0.006). These data demonstrate the value of triplex-induced apoptosis as a therapeutic alternative for drug resistant cancers with copy number gains.

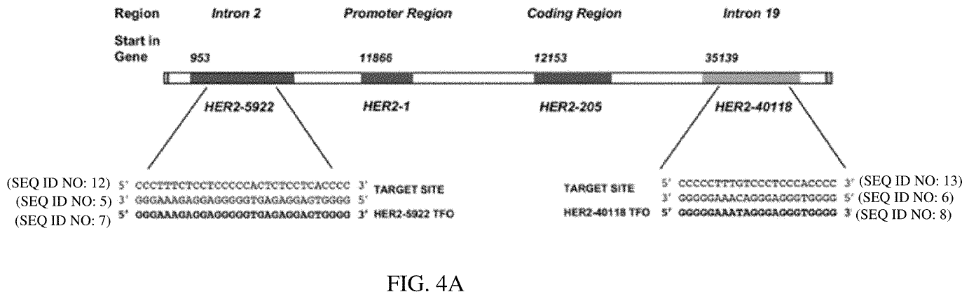

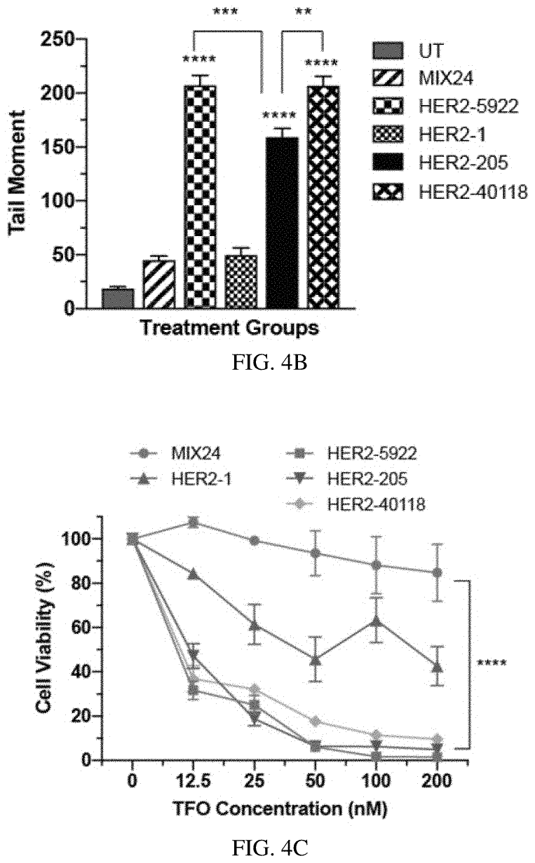

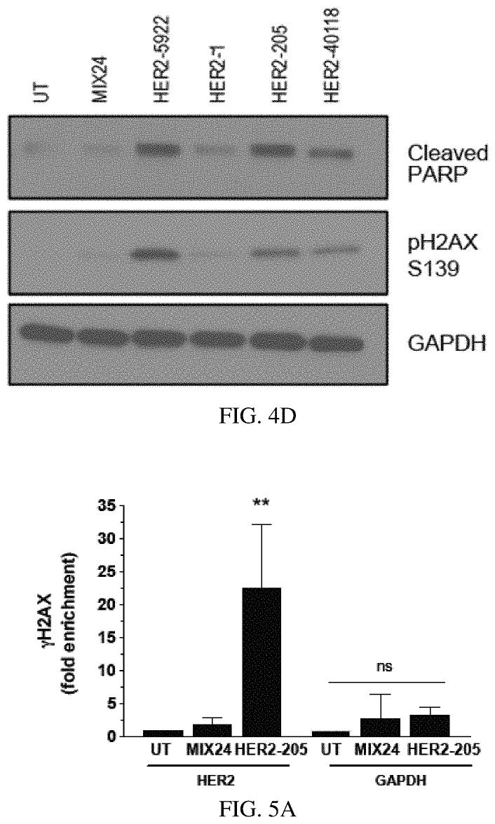

In humans, there is at least one unique and high affinity triplex targeting site located in the promoter and transcribed regions of each protein-coding gene. Mapping of polypurine sequences with characteristics to serve as a potential target site has identified 519,971 unique sequences throughout the human genome²³. In order to further investigate the versatility of the approach, TFOs were designed to target sites within other regions of the HER2 gene (FIG. 4A). HER2-5922 was designed to target a polypurine sequence in intron 2 and HER2-40118 was directed to a sequence within intron 19 (FIG. 4A). The TFOs were first assessed for their ability to induce DNA damage, compared to TFOs targeting the promoter and coding regions. It was determined by neutral comet assay that HER2-5922 and HER2-40118 were more effective at inducing DNA damage than HER2-205 as indicated by an increase in DNA tail moment (FIG. 4B). BT474 cells were then exposed to increasing concentrations of the HER2-targeted TFOs. As shown in FIG. 4C, cell viability decreased with increasing TFO concentrations, with HER2-205, HER2-5922 and HER2-40118 exhibiting similar dose responses with ˜50% cell death at a concentration of 12.5 nM. Western blot analysis of cleaved PARP confirmed triplex-induced apoptosis that corresponded to an increase in DSBs as indicated by H2AX phosphorylation at S139 (FIG. 4D). These results solidify the feasibility of this therapeutic strategy and emphasize that every amplified cancer driver gene should have multiple polypurine sequences that can be targeted using gene-specific bioactive TFOs.

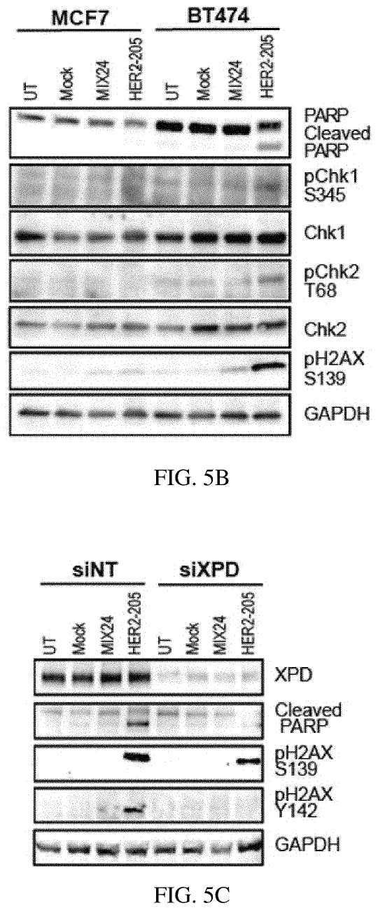

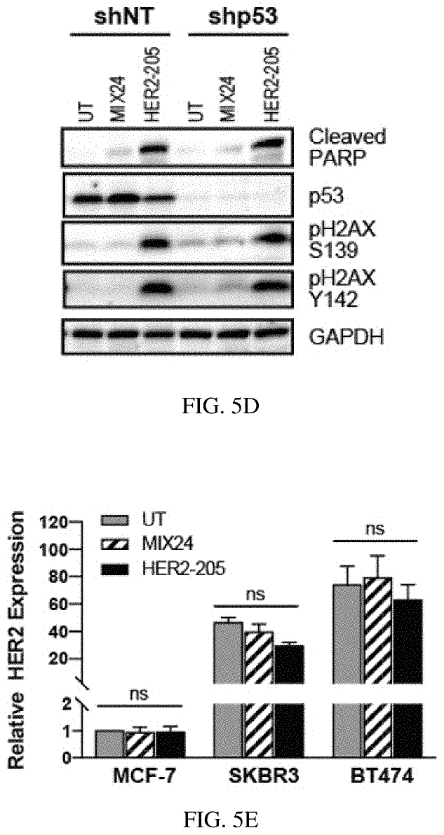

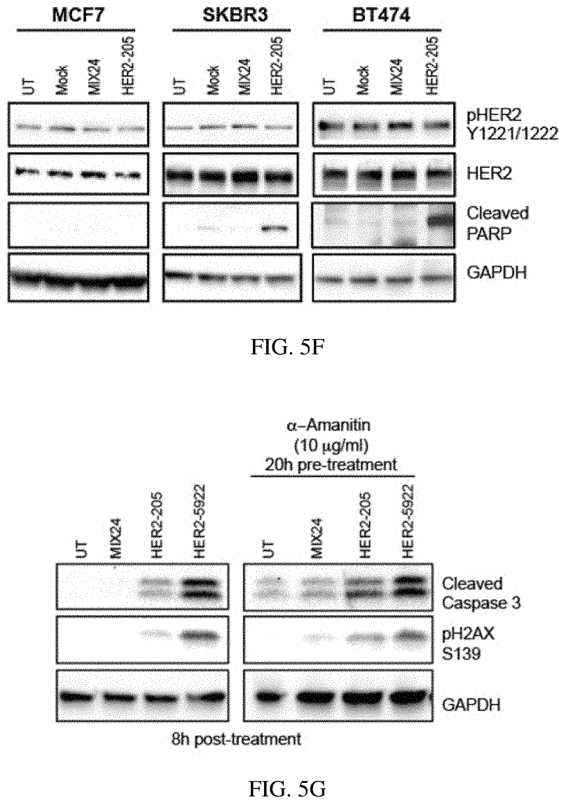

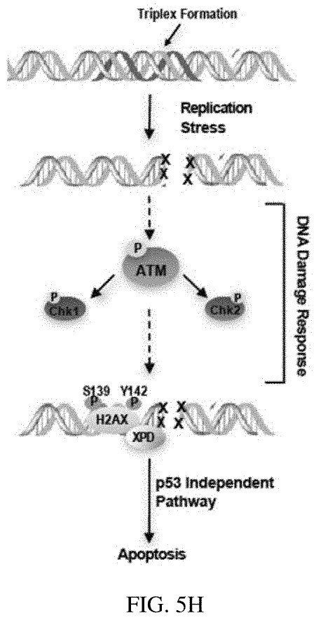

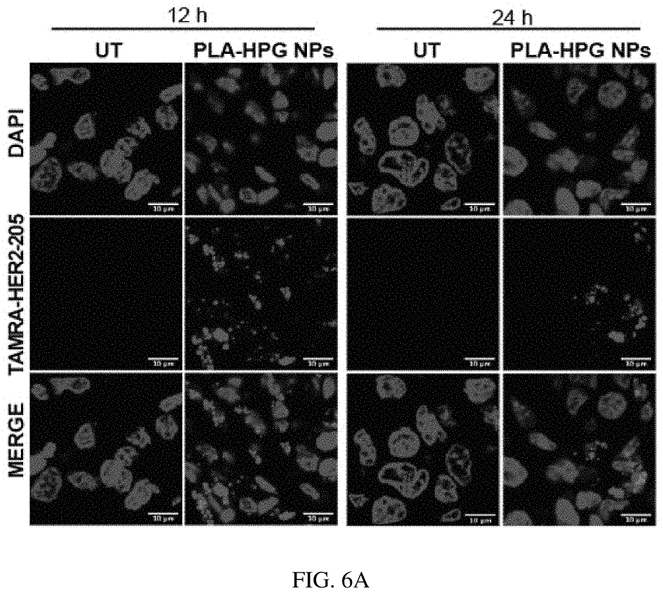

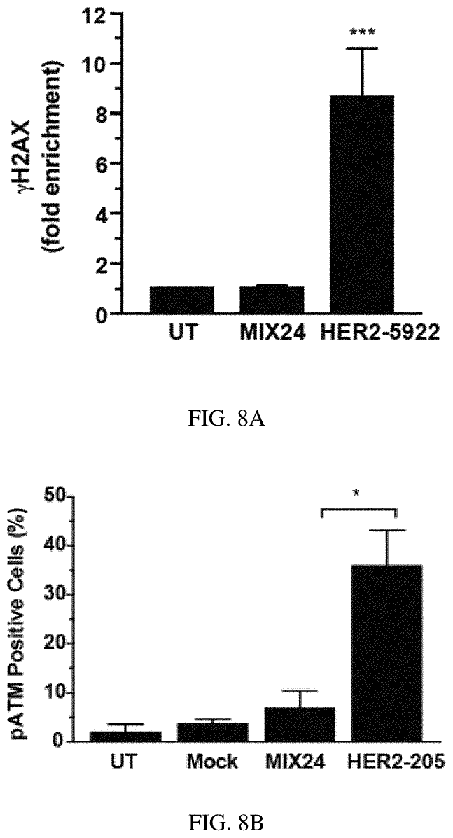

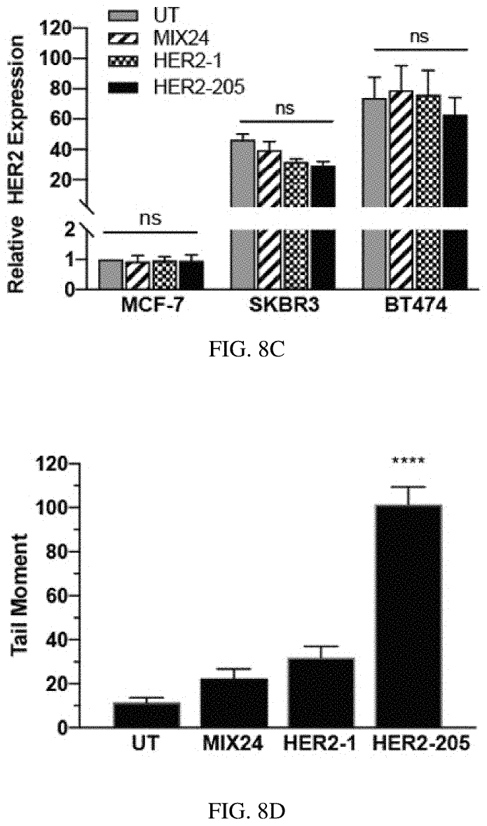

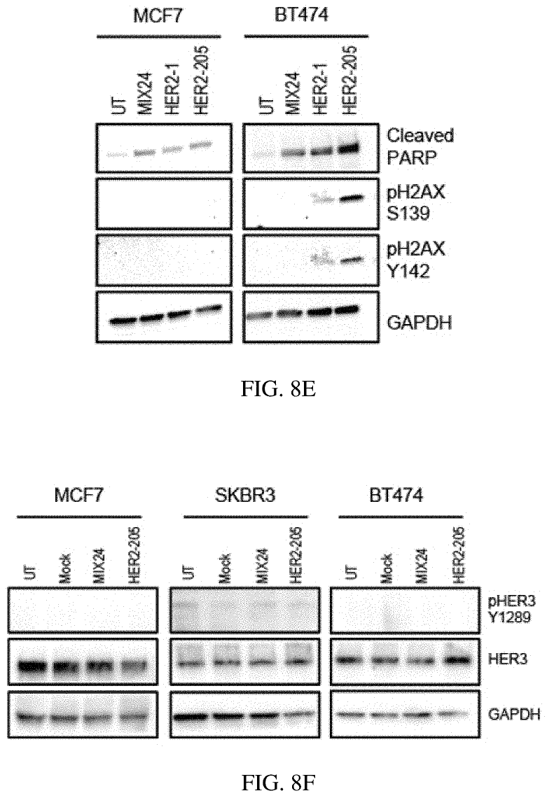

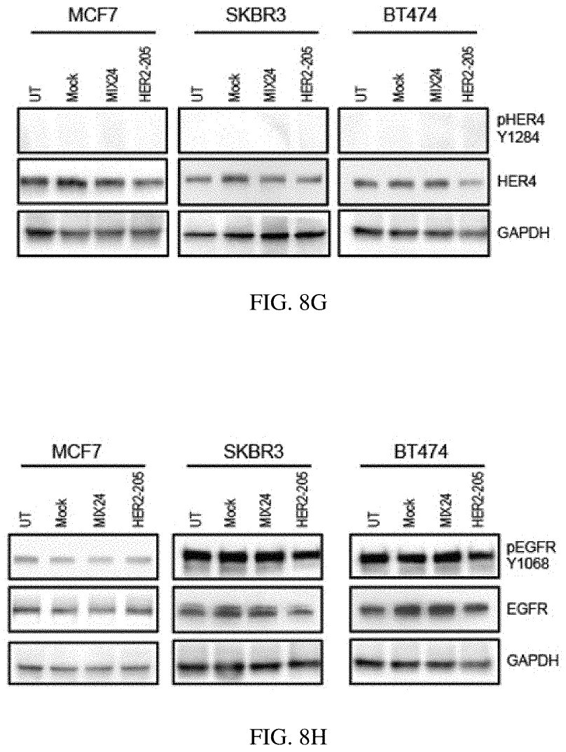

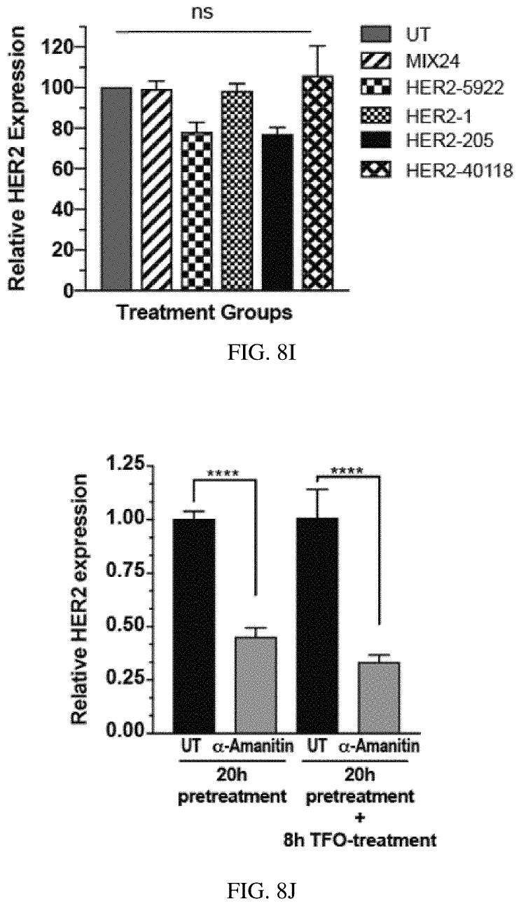

To define the mechanism of drug action and characterize the DNA damage response activated in TFO-treated cells, target specific induction of DNA damage by HER2-205 was confirmed using chromatin immunoprecipitation (ChIP) assays for γH2AX and multiplexed qPCR with a probe for the HER2 gene locus. A 22-fold enrichment of γH2AX was detected at the HER2 gene relative to untreated cells 8 h post TFO-treatment (FIG. 5A). Moreover, analysis for the induction of DNA damage at a non-targeted region of the genome using a probe for the GAPDH gene locus did not detect the presence of γH2AX above background levels following HER2-205 treatment (FIG. 5A). Furthermore, targeting of the intron with HER2-5922 also resulted in gene-specific induction of DNA damage (FIG. 8A). These findings support a mechanism that TFO-generated structures can induce DNA damage specifically at the targeted amplified oncogenic gene locus.

Next, the status of ATM, Chk1/Chk2 and the NER factor, XPD in HER2 positive cells following HER2-205 treatment was determined. As shown in FIG. 5B, Chk1 phosphorylation at serine 345 was observed after HER2-205 treatment in the HER2-amplified cells and not in the cells with normal HER2 gene copy numbers. Chk1 activation in BT474 cells corresponds to induction of DSBs and apoptosis as determined by Western blot analysis of pH2AX S139 and cleaved PARP, respectively. In addition, phosphorylation of Chk2 at threonine 68 was observed in response to triplex-induced DSBs in the BT474 cells (FIG. 5B). These phosphorylation events correspond to an increase in pATM positive cells following HER2-205 treatment (FIG. 8B).

Regulation of the phosphorylation status of H2AX at tyrosine 142 (Y142) is crucial for determining the recruitment of either DNA repair or pro-apoptotic factors to the DSB site²⁴. It has been found that H2AX Y142 is phosphorylated in response to HER2-205 induced DSBs to trigger apoptosis as indicated by Western blot analysis of cleaved PARP (FIG. 5C). XPD occupies a central role in the mechanism that modulates survival/death decisions in response to triplex-induced DNA damage¹⁸. Accordingly, a requirement for XPD in the phosphorylation of Y142 in H2AX and activation of apoptosis following HER2-205 treatment was seen (FIG. 5C). These results suggest that the absence of XPD disrupts the signaling pathway used to activate apoptosis following TFO treatment and support a mechanism of action that is dependent upon DNA damage response.

The p53 tumor suppressor regulates pro-apoptotic pathways in response to severe DNA damage. However, over 50% of human cancers exhibit chemotherapeutic resistant phenotypes due to loss of function p53 mutations which lead to an inability to trigger apoptosis. To test whether triplex-induced DNA damage could activate p53-depleted apoptosis, p53-depleted BT474 cells were treated with HER2-205. It was found that TFO-treatment of p53-depleted cells results in a similar level of PARP cleavage compared to treatment of control cells, confirming that triplex formation can activate apoptosis irrespective of p53 status (FIG. 5D). Unlike XPD-depleted cells, which displayed a decrease in TFO-induced apoptosis, it was also demonstrated that triplex-induced DSBs trigger robust H2AX Y142 phosphorylation in the absence of p53 (FIGS. 5C, 5D).

Trastuzumab's anticancer activity has been attributed in part to changes in HER2 tyrosine phosphorylation and a reduction in total HER2 protein^{25,26}. To further demonstrate that HER2-205 activity is independent of the cellular function of HER2, HER2 gene expression was analyzed by RT-PCR (FIG. 5E) and monitored total HER2 protein and phosphorylation levels by Western blot following treatment in several breast cancer cell lines (FIG. 5F). The results showed that HER2 gene expression is not significantly affected by HER2-205 treatment in either the non-amplified or amplified breast cancer cell lines (FIG. 5E) and that total and activated HER2 levels remain the same following triplex-induced apoptosis in the HER2-positive cells compared to the control samples (FIG. 5F). Although a significant impact on HER2 gene expression following HER2-1 treatment was not detected, significantly more TFO-induced DNA damage and activation of apoptosis with HER2-205 was observed compared to HER2-1, further supporting a mechanism that activity is dependent on the ability to induce DNA damage and not disruption of gene expression (FIGS. 8C-8E). In general, no changes were noted in the levels of HER3, HER4 and EGFR following HER2-205 treatment compared to the untreated or MIX24 treated cells (FIGS. 8F-8H). Additionally, targeting in the introns did not result in a significant decrease in HER2 gene expression following treatment with either HER2-5922 or HER2-40118 (FIG. 8I).

In order to investigate whether active transcription was a prerequisite for TFO induced DNA damage and activation of apoptosis, transcription was inhibited in BT474 cells with α-amanitin prior to HER2-205 treatment (FIG. 8J). The results demonstrate similar increases in the levels of γH2AX and cleaved caspase 3 compared to controls following TFO treatment in cells with and without transcription inhibitor pretreatment (FIG. 5G), suggesting that active transcription is not required for TFO-induced DNA damage or activation of apoptosis. Taken together, these results support a mechanism of action for the HER2-targeted TFO that is independent of the cellular function of HER2 (FIG. 5H).

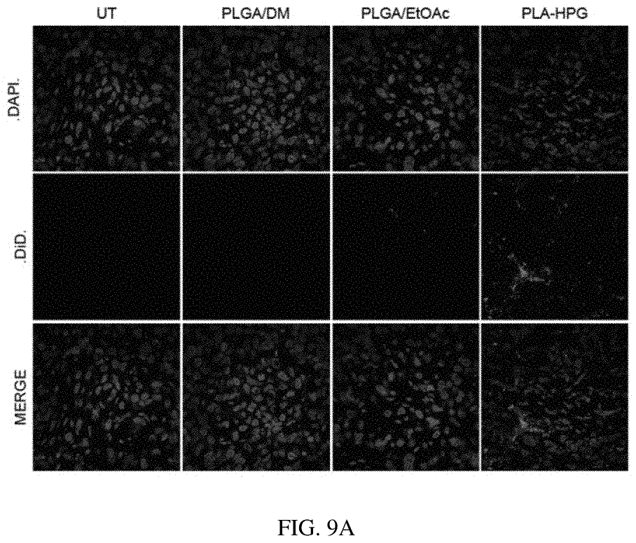

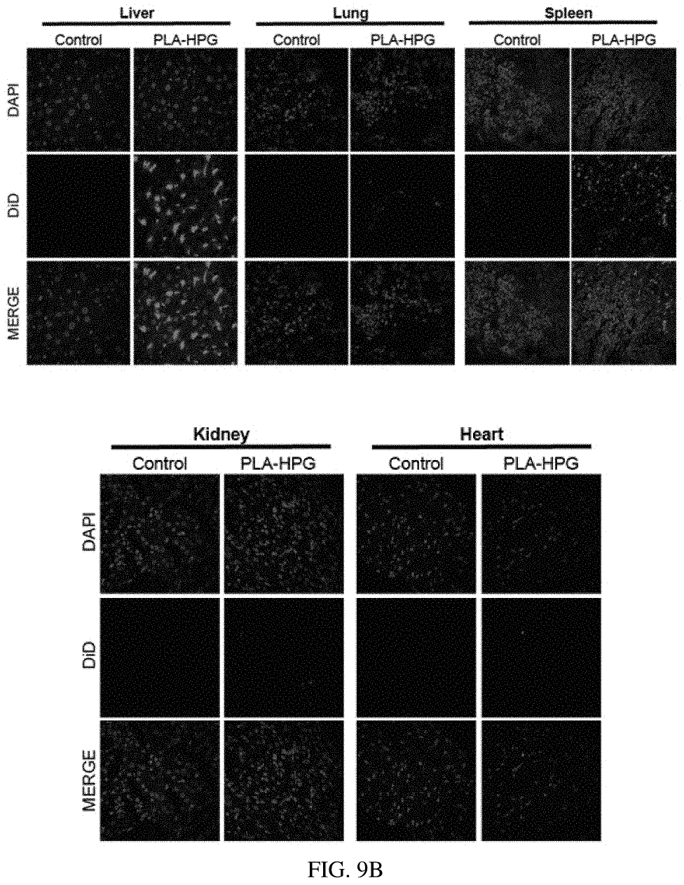

Inadequate drug delivery to the intended disease site can significantly impact therapeutic effect. It was hypothesized that polymeric, biodegradable nanoparticles (NPs) could serve as a delivery platform and enhance drug efficacy of the HER2-targeted TFOs. Several NPs formulations were evaluated for their tumor delivery potential by screening for fluorescent dye uptake using DiD (FIGS. 9A-9B). DiD was encapsulated using two NP formulations fabricated from poly(lactic-co-glycolic acid) (PLGA), a polymer that has been approved by the FDA for numerous drug delivery applications. In addition, NPs were screened from a copolymer of poly(lactic acid) (PLA) and hyperbranched polyglycerol (HPG), PLA-HPG, which has previously demonstrated long blood circulation times and effective tumor uptake²⁷. It was determined that DiD-loaded PLA-HPG NPs had more efficient tumor uptake compared to the PLGA formulations following intravenous administration by retro-orbital (RO) injection in an orthotopic mouse model of BT474 breast cancer cells (FIG. 9A). To better understand the overall impact of PLA-HPG NPs delivery, biodistribution in the liver, lung, spleen, kidney and heart was evaluated 12 h post administration (FIG. 9B).

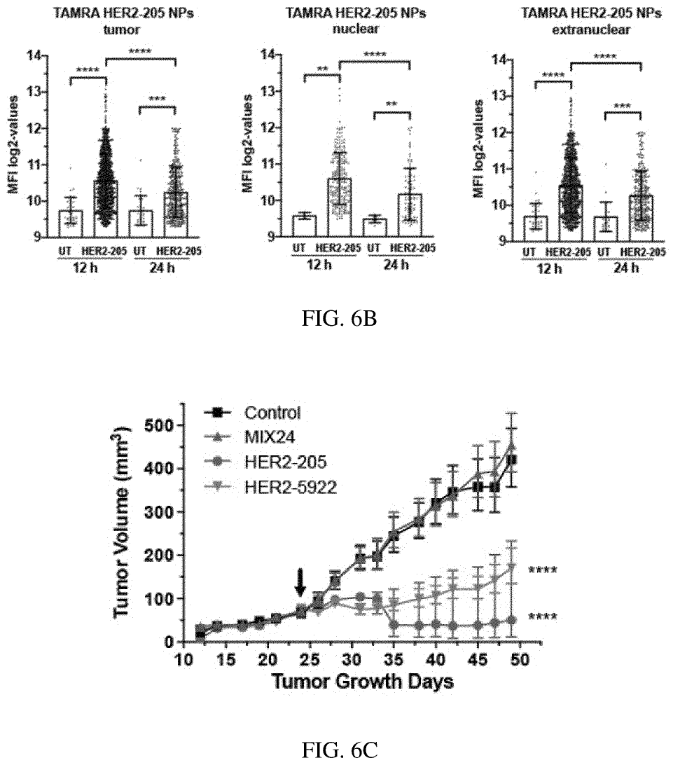

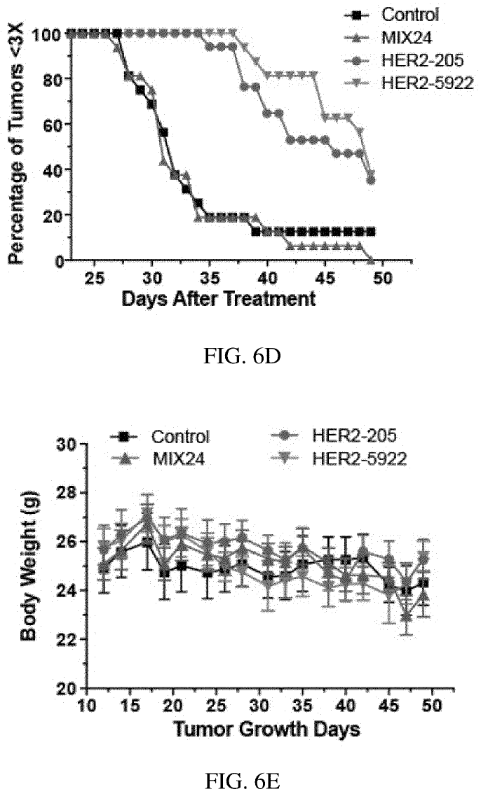

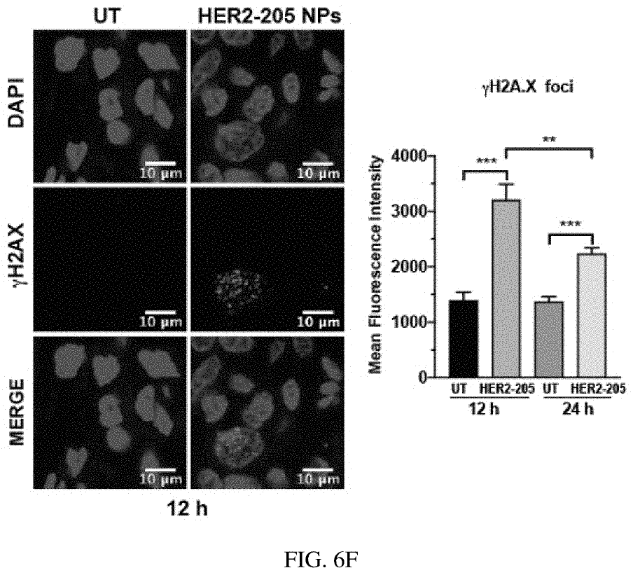

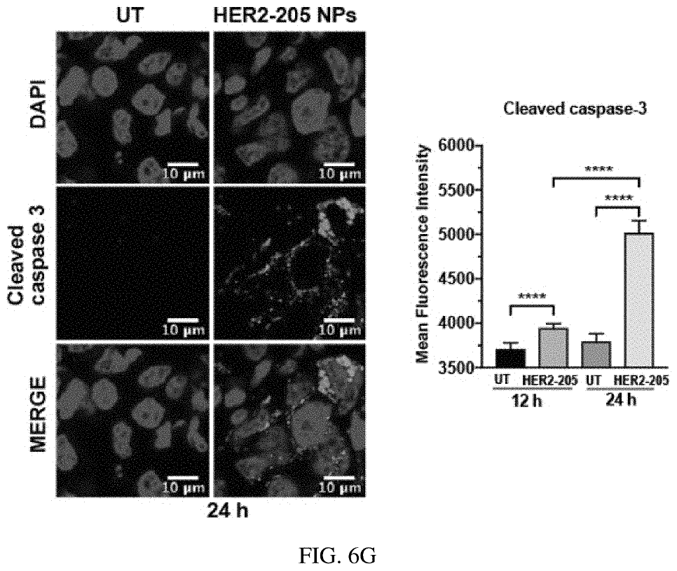

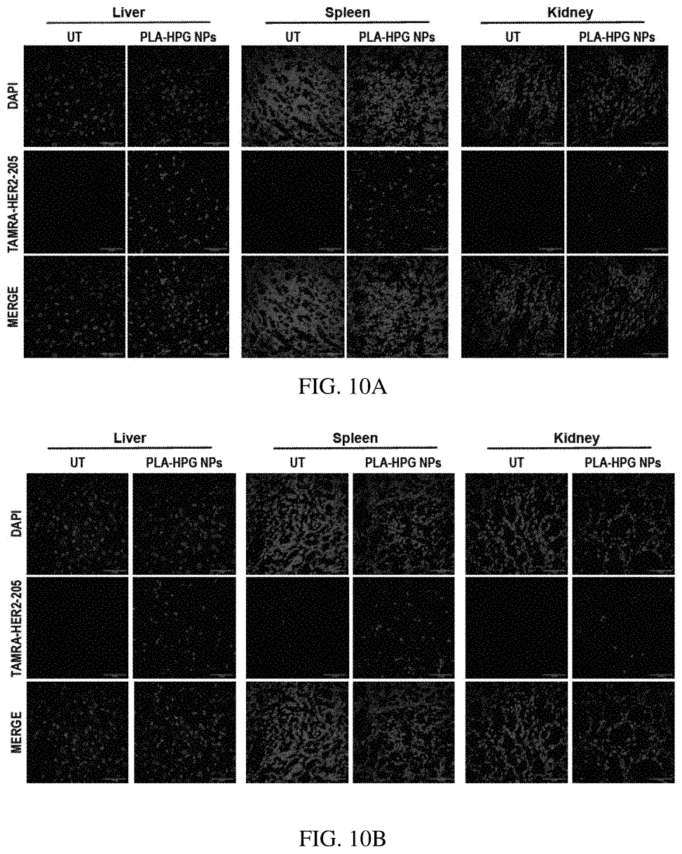

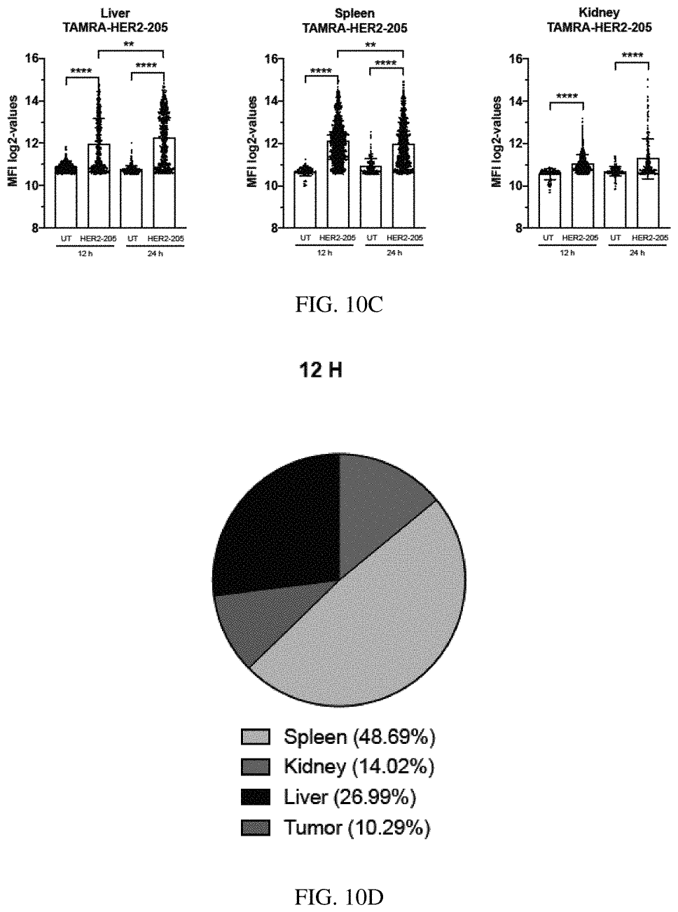

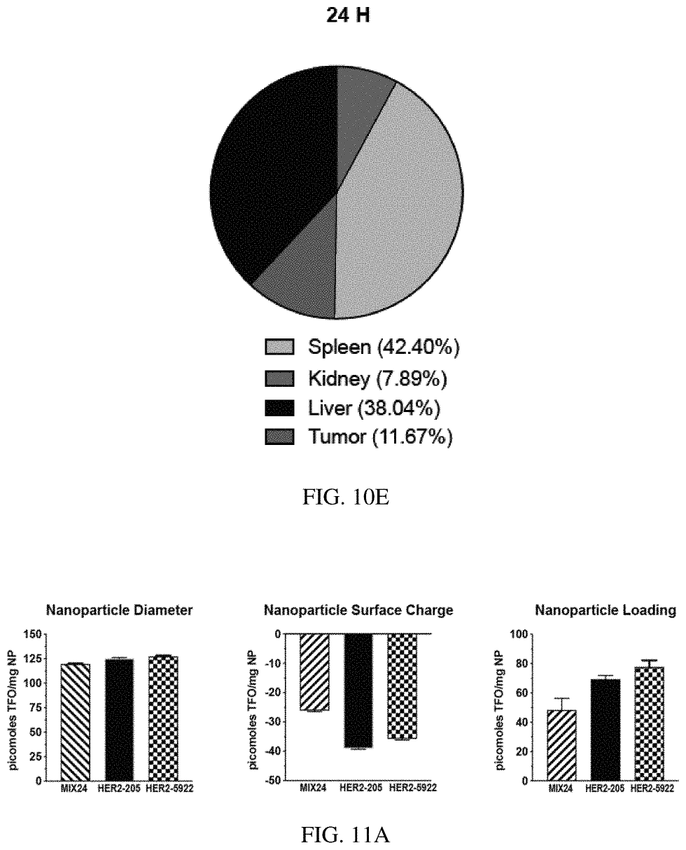

To visualize TFO uptake and inform timing and dosing for subsequent tumor growth studies, TAMRA-HER2-205 was encapsulated in PLA-HPG NPs and tumor distribution was evaluated 12 and 24 hours post systemic administration in mice bearing orthotopic BT474 breast cancer tumors (FIG. 6A). To confirm that cells internalized HER2-205, confocal microscopy was performed on tumor tissue and quantified nuclear and extranuclear TAMRA fluorescence. Administration of the NPs resulted in TAMRA-HER2-205 accumulation in the tumor, which is detectable at 12 hours with a slight reduction in fluorescence intensity 24 hours post treatment (FIG. 6B). Furthermore, PLA-HPG NPs delivered TAMRA-HER2-205 to the nucleus, with significantly greater fluorescence being detected at 12 h post-treatment compared to 24 h (FIG. 6B). The fluorescence intensity of TAMRA-HER2-205 in the liver, kidney and spleen compared to tumors was also evaluated in order to determine TFO biodistribution at 12 and 24 hours post treatment (FIGS. 10A-10E). Administration of TAMRA-HER2-205 PLA-HPG NPs resulted in ˜10% distribution of the TFO in the tumor at both time points (FIGS. 10D, 10E). As observed in the biodistribution studies using DiD, higher uptake was detected in the liver and spleen (27% and 49%, respectively at 12 hours post-treatment) compared to the tumor (FIGS. 10D, 10E).

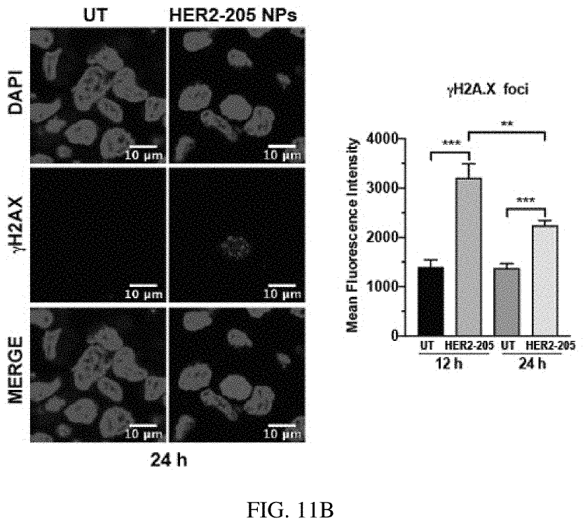

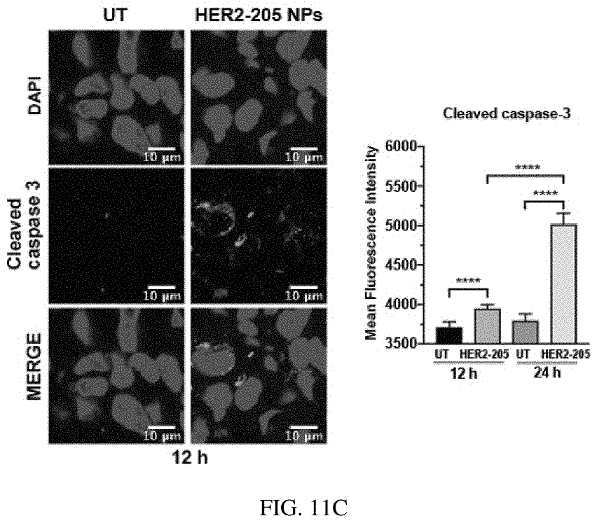

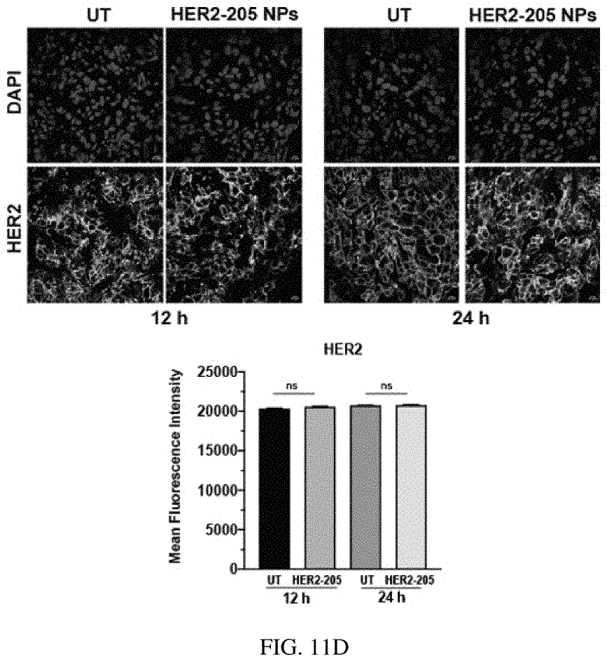

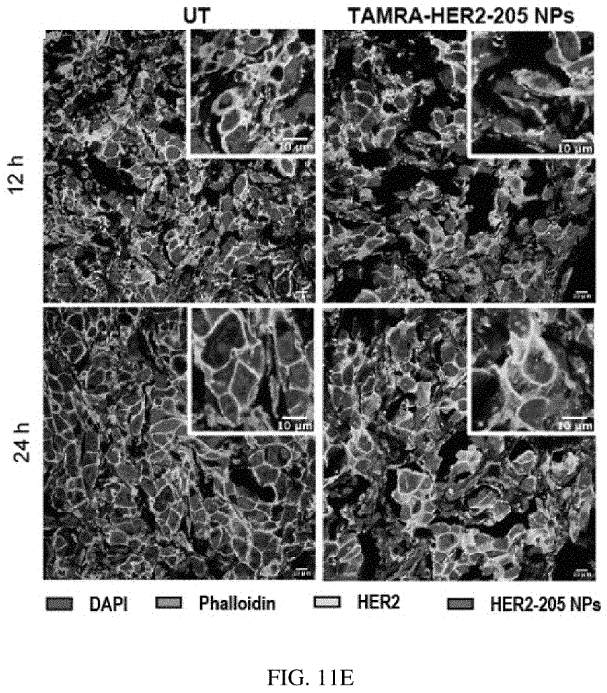

The impact of the PLA-HPG delivery system (FIG. 11A) on therapeutic efficacy was assessed, using an orthotopic mouse tumor xenograft model of BT474 breast cancer cells. In this model, systemic administration of HER2-205 and HER2-5922 PLA-HPG NPs significantly decreased tumor growth and improved survival (as defined by tumor volume 3× that of initial treatment) (FIGS. 6C, 6D). As observed in other models, MIX24 PLA-HPG NPs did not alter tumor growth or survival as compared with untreated controls (FIG. 6C). The efficacy of this therapeutic strategy is not limited by the target region, as HER2-5922 NP treatment resulted in a significant delay in tumor growth (FIG. 6C). A tumor tripling time of 45±1.5 days post-initial dose was observed in tumors treated with HER2-205 NPs and 47±1.2 days with HER2-5922 NPs compared to 33±1.4 days with MIX24 NPs (FIG. 6D). In contrast, MIX24 NPs had no impact on BT474 tumor growth relative to untreated controls (31±0.8 2 days). Notably, when compared to treatment with naked oligonucleotides, the TFO encapsulated PLA-HPG NP delivery system reduced by more than 99% the amount of TFO (˜80 nmol/dose with naked TFO compared to ˜0.14 nmol/dose with NPs) required to have a significant impact on tumor growth delay and improved survival. Additionally, no gross toxicity, including weight loss was noted in mice treated with the HER2-targeted TFOs NPs (FIG. 6E).

To investigate the mechanism underlying efficacy, BT474 tumor-bearing mice were treated with HER2-205 NPs (2 mg, RO injection) and collected tumors for immunofluorescence analysis 12 and 24 hours after treatment. As compared with untreated controls, tumors treated with HER2-205 had significantly higher levels of γH2AX foci (2-fold increase as measured by MFI), confirming that HER2-targeted TFOs were capable of inducing DNA damage in tumors (FIGS. 6F and 11B). To determine whether this degree of DNA damage was sufficient to induce cell death, the activation of caspase 3 as a biomarker for apoptosis was also investigated (FIGS. 6G and 11C). A significant increase in apoptosis was observed 24 h following HER2-205 treatment compared to untreated tumors (FIG. 6G), which corresponded to the increase in DNA damage observed at 12 h (FIG. 6F). Importantly, similar to the mechanistic studies in cells, a decrease in HER2 protein levels was not observed in the HER2-205 treated tumors compared to tumors from untreated mice (FIGS. 11D, 11E). Together, these results indicate that HER2-205 exhibits therapeutic efficacy utilizing a mechanism independent of HER2 cellular function and based on DNA damage response, where induction of DNA damage activates apoptosis.

### Discussion

The compositions and methods disclosed herein constitute a new paradigm for targeted therapeutics, efficacious in the treatment of cancers driven by gene amplification with minimal potential for toxicity to normal tissue. Also described herein are agents with a unique mechanism of action and results that show that induction of the DNA damage response via TFO treatment is as effective as targeting the overexpressed oncogenic protein product. HER2-205 treatment of HER2-positive breast cancer xenografts resulted in a 52% reduction in tumor volumes compared to controls, which is comparable to the 58% reduction observed with trastuzumab. Enhanced tumor delivery (delivery to a tumor) using a NP platform can significantly improve the efficacy of TFO-treatment. Furthermore, development of bioactive reagents is not restricted to polypurine sequences within a specific region of the amplified gene. The compositions and methods disclosed herein can be used as a drug design platform and treatment option for several cancers with gene amplification and resistance to current therapies.

Described herein is a method of reducing, in a population of cells, the number of cancer cells, such as p53-depleted cancer cells, in which a HER2 gene is amplified and agents useful to reduce the number of cancer cells, such as p53-depleted cancer cells, comprising an amplified HER2 gene. In specific embodiments, the agents are triplex-forming oligonucleotides (TFOs) that are targeted to a polypurine site in an amplified HER2 gene in p53-depleted cancer cells.

In one embodiment, the method comprises contacting a population of cells, such as tissue, comprising cancer cells, such as p53-depleted cancer cells, in which a HER2 gene is amplified with triplex forming oligonucleotides (TFOs) targeted to a polypurine target site in the HER2 gene(s), under conditions under which the TFOs enter the cancer cells in sufficient quantity to induce apoptosis.

Advancements in DNA sequencing technology have not only revealed commonly mutated and deleted genes across cancer types, but also enabled identification of amplified cancer-promoting genes⁸. These amplified genes include epigenetic regulators, cell cycle-associated genes, and genes linked to signaling pathways, such as the EGFR and HER2 genes⁹. Described herein is an approach for targeted therapeutics that can be used in the treatment of cancers characterized by gene amplification and that has limited toxicity to normal tissue. The limited toxicity is at least due to the localized effect and targeting of the TFOs to cells that have HER2-amplified genes and thus are likely to be the cancerous cells in the tissue. Described herein are agents and methods demonstrating that manipulation of DNA damage response is as effective in its anticancer activity as targeting the individual overexpressed protein product.

Provided herein are triplex-forming oligonucleotides (TFOs, also referred to as triplex-inducing oligonucleotides) for the induction of p53-independent apoptosis. TFOs are molecules that function as sequence-specific gene targeting/modification tools. Without wishing to be bound by theory, it has been shown that the TFOs bind to the major groove of duplex DNA and are restricted to sites with purines (also referred to as polypurine sites) on one strand and pyrimidines on the other.

### HER2 Gene Amplification and NER-Dependent Repair

Gene amplification is observed in a broad spectrum of cancers, contributing not only to incipient cancer development, but also to the development of drug resistance. HER2 gene amplification (amplification of human epidermal growth factor receptor 2-encoding gene) is observed in a vast majority of cancers. Cancers with HER2 gene amplification or overexpression of the HER2 protein are sometimes referred to as HER2-positive cancers. Non-limiting examples of such cancers include breast cancer, ovarian cancer, colorectal cancer, gastric cancer, lung cancer, and endometrial cancer. HER2 gene amplification has been identified in about 25% of breast cancers.

Disclosed herein are TFOs targeted to specific regions of the HER2 gene. These TFOs can be utilized for a p53-independent cancer therapy. There are several polypurine sites in the HER2 gene that are susceptible to triplex formation. Binding of TFOs to the HER2 gene (e.g., major groove regions on the HER2 gene) causes DNA perturbation that can impede replication fork progression, resulting in fork collapse and helix distorting structures (e.g., lesions or, more specifically, DNA double strand breaks (DSBs))¹⁷. Under normal circumstances (e.g., low HER2 gene copy levels), these helix distorting structures trigger the nucleotide excision repair (NER) pathway, which repairs the helix distorting structures. This ability of the NER pathway to resolve low levels of triplex-induced DNA damage allows normal cells to tolerate the formation of a few triplexes^{19,20}. In contrast, if there is HER2 gene amplification and consequently high levels of triplex formation, NER-dependent DNA repair is ineffective and instead apoptosis is triggered¹⁸. HER2 gene amplification in cancers, such as breast cancers, provides an opportunity to test the efficacy of TFOs as an apoptosis-inducing agent in cancer cells, but not in healthy cells, which lack HER2 amplification18 (FIGS. 1A-1C).

XPD, a transcription factor II H (TFIIH) subunit, plays a key role in this NER pathway by operating as a 5′-3′ helicase to unzip the DNA. In instances of high DNA damage (or high triplex formation), XPD is required for p53-mediated apoptosis (see U.S. Pat. No. 9,587,238, the relevant disclosures of which are herein incorporated as reference). Previous studies established that in cases of excess DNA damage, an apoptotic pathway is initiated that is dependent on the presence of both XPD and p53. This, in part, explains the chemotherapeutic drug resistance and the difficulty in treating p53-defective conditions.

### P53 Tumor Suppressor

Disclosed herein are methods and compositions for inducing apoptosis in p53-depleted cells comprising an amplified HER2 gene. P53 (also referred to as TP53 or p53 tumor suppressor) is a gene on the 17th chromosome (17p13.1) that encodes p53 protein (also referred to as TP53 or tumor protein). The protein is a regulator in the cell cycle and plays the role of a tumor suppressor. The p53 tumor suppressor regulates pro-apoptotic pathways in response to severe DNA damage. Under normal, non-pathological conditions, p53 expression is low. DNA damage and related signals upregulate its expression to initiate growth arrest, DNA repair, and, in extreme cases, apoptosis. Typically, growth arrest inhibits replication of damaged DNA; however, in cancerous cells this is bypassed. As explained herein, gene amplification manifests in cancerous cells and can result in ineffective DNA repair (for example, ineffective NER-mediated DNA repair). In such cases, p53 is relied on for apoptosis of the damaged cells.

Mutations in p53 are correlated with a broad spectrum of aggressive cancers and have been implicated in as many as 50% of all human tumors, highlighting the importance of this gene and the impact of a p53-independent chemotherapeutic approach. Over 50% of human cancers exhibit chemotherapeutic resistant phenotypes due to loss of function p53 mutations, which lead to an inability to trigger apoptosis. Previous studies attempted to address cancer treatment in p53-defective conditions by upregulating wild-type p53 or augmenting the activity of wild-type p53 (Smith and Seo, Mutagenesis 17(2), 149-156, 2002). Additional strategies to overcome this challenge include attempts to modify the p53 gene through gene editing, reactivate p53 genes with chemotherapeutic drugs, or suppress p53 mutant aggregation. These strategies have had limited success.

The term “p53-depleted” refers to cells, such as cancer cells, in which p53 is reduced, lacking or mutated. In some embodiments, a p53-depleted cancer cell is a cell that does not express p53. It can also refer to significantly decreased p53 expression under conditions under which p53 is typically upregulated (e.g., in response to a DNA lesion). Mutations in p53 have been shown to give rise to different isoforms, some of which give rise to tissue-specific cancers. The term “p53-depleted” also includes cells, such as cancer cells, in which the p53 gene has a mutation (e.g., loss of function mutation, gain of function mutation, etc.) and produces a protein that is dysfunctional (e.g., displays no or reduced function). This may also occur through the production of a truncated p53 protein that is dysfunctional. Most p53 mutations are missense mutations. In some embodiments, the p53-mutated cell is a homozygote mutant. In some embodiments, the p53-mutant cell is a heterozygote, carrying a wild-type p53 allele and a mutant p53 allele. Previous studies have shown that in some heterozygote cases, the mutant allele functions in a dominant negative manner, suppressing the expression of the wild-type allele. Loss of wild-type p53 and p53 mutations have been shown to occur in both early and late tumorigenesis. Some p53 mutations, referred to as gain-of-function p53 mutations, result in p53 mutant proteins that have additional oncogenic properties and promote cancer progression (Rivlin et al., Genes & Cancer 2(4):466-474, 2011).

### Triplex Forming Oligonucleotides (TFOs)

Triplex-forming oligonucleotides (TFOs) form triplexes, which are DNA structures comprised of an additional RNA or DNA binding sequence. Without wishing to be bound by theory, they are believed to bind in the major groove of duplex DNA. Purine motif TFOs (comprised of G and A) form G*G:C and A*A:T triplets and bind in antiparallel orientation, via reverse Hoogsteen base pairing, with regard to the purine strand of the duplex. In contrast, pyrimidine motif TFOs (C/T) form triplexes in parallel orientation, via forward Hoogsteen alignment, and form C+*G:C and T*A:T triplets. Mixed purine and pyrimidine TFOs bind in either parallel or antiparallel orientation and form G*G:C and T*A:T triplets. (Maldonado, R., et al. RNA 24(3): 371-380, 2018 and Basye, J., et al. Nucleic acids research 29(23): 4873-4880, 2001).

### Applications of HER2 Targeted TFOs

As described, HER2-targeted TFOs trigger an alternative pathway, a p53-independent apoptotic pathway. HER2-targeted TFOs induce copy number dependent DNA double strand breaks (DSBs) and activate apoptosis in HER2 gene amplified cancer cells and human tumor xenografts via a mechanism that is independent of HER2 cellular function as well as independent of p53. In specific embodiments, HER2-targeted TFOs, HER2-1 (SEQ ID NO:3), HER2-205 (SEQ ID NO:4), HER2-5922-2 (SEQ ID NO:7), and HER2-40118 (SEQ ID NO: 8), trigger p53-independent apoptosis in cancer cells comprising amplified HER2 gene.

In some embodiments, the HER2-targeted TFOs target polypurine target sites in the promoter region of the HER2 gene (e.g., the HER2-1 TFO (SEQ ID NO: 3)). In some embodiments, the HER2-targeted TFOs target polypurine target sites in the coding region of the HER2 gene (e.g., the HER2-205 TFO (SEQ ID NO: 4)). In some embodiments, the HER2-targeted TFOs target introns or non-coding regions of the HER2 gene. For example, the HER2-5922-2 TFO (SEQ ID NO: 7) targets a site within intron 2 of the HER 2 gene, and the HER2-40118 (SEQ ID NO: 8) TFO targets intron 19 of the HER2 gene.

Disclosed herein is a method of reducing, in a population of cells, the number of p53-depleted cancer cells comprising an amplified HER2 gene. As described, this method can be carried out in a population of cells, such as in a tissue or organ. As used herein, the term “reducing” refers to decreasing the number of living cells by inducing apoptosis in the cells. The reduction could decelerate rapid cell growth or decelerate hyperplasia, which are two common characteristics of cancerous cells.

The methods disclosed herein includes contacting p53-depleted cancer cells comprising amplified HER2 gene with triplex-forming oligonucleotides (TFOs) targeted to a polypurine target site in the amplified-HER2 gene.

In some embodiments, the TFOs are polypurine TFOs. Polypurine TFOs are rich in adenine and/or guanine bases, and, without wishing to be bound by theory, are believed to bind to the major groove of their polypurine target sites in an antiparallel fashion. As used, the term “polypurine TFO” refers to purine motif TFOs or TFOs rich in purines (adenine and/or guanine bases). As used, the term “polypurine target site” refers to a DNA duplex having a strand rich in purines (adenine and/or guanine bases). The terms “polypurine target strand” and “polypurine strand” refer to the strand in the polypurine target site that is rich in purines (adenine, guanine or both adenine and guanine bases). In some embodiments, a sequence is referred to as “rich in purines” when 50%, 55%, 60%, 65%, 70%, 75%, 80%, 85%, 90%, 95%, 98%, 99% or more of its nucleotides have adenine and/or guanine bases.

An example of a polypurine target site identified in the promoter region of the HER2 gene is a DNA duplex having the sequence 5′-AGGAGAAGGAGGAGGTGGAGGAGGAGGG-3′ (SEQ ID NO:1) bound to 5′-CCCTCCTCCTCCACCTCCTCCTTCTCCT-3′ (SEQ ID NO:10). (FIG. 1D). Another example of a polypurine target site identified in the coding region of the HER2 gene is a DNA duplex having the sequence 3′-CCCCGAGGAGGAGCGGGAGΔΔCGGGGGG-5′ (SEQ ID NO:2) bound to 5′-GGGGCTCCTCCTCGCCCTCTTGCCCCCC-3′ (SEQ ID NO:11). (FIG. 1D).

Another example of a polypurine target site identified in the non-coding region of the HER2 gene is a DNA duplex having the sequence 3′-GGGAAAGAGGAGGGGGTGAGAGGAGTGGGG-5′ (SEQ ID NO: 5) bound to 5′-CCCTTTCTCCTCCCCCACTCTCCTCACCCC-3′ (SEQ ID NO: 12). (FIG. 4A). Another example of a polypurine target site identified in the non-coding region of the HER2 gene is a DNA duplex having the sequence 3′-GGGGGAΔΔCAGGGAGGGTGGGG-5′ (SEQ ID NO: 6) bound to 5′-CCCCCTTTGTCCCTCCCACCCC-3′ (SEQ ID NO: 13). (FIG. 4A).

Formation of the triplex after introduction of a TFO occurs via reverse Hoogsteen hydrogen bonds between the third strand (TFO) and the polypurine strand of the duplex.

In some embodiments, a TFO has a nucleotide sequence that is complementary to SEQ ID NOs: 10, 11, 12 and 13 and/or binds to at least 13 nucleotides in a polypurine target strand, such as at least 13 nucleotides in SEQ ID NO:1, SEQ ID NO:2, SEQ ID NO: 5 or SEQ ID NO: 6. In some embodiments, a TFO binds to 13, 14, 15, 16, 17, 18, 19, 20, 21, 22, 23, 24, 25, 26, 27, 28, 29 or 30 nucleotides in a polypurine target strand. TFOs can bind to contiguous or non-contiguous nucleotides in a polypurine target site. The TFOs described herein can be any TFO sequence that is targeted to a polypurine target site in a HER2 gene, for example, polypurine target sites SEQ ID NO:1, SEQ ID NO:2, SEQ ID NO: 5 or SEQ ID NO: 6. In some embodiments, the TFOs are at least 13 nucleotides in length. In some embodiments, the TFOs range from 13 to 30 nucleotides in length. For example, the TFOs can be 13, 14, 15, 16, 17, 18, 19, 20, 21, 22, 23, 24, 25, 26, 27, 28, 29, or 30 nucleotides in length. In further embodiments, the TFOs are shorter (e.g., 8, 9, 10, 11, or 12 nucleotides).

Examples of HER2-targeted TFOs are HER2-1 (5′-GGGAGGAGGAGGTGGAGGAGGAAGAGGA-3′; SEQ ID NO:3), HER2-205 (5′-GAGGAGGAGTGGGAGAATGGGGGG-3′; SEQ ID NO:4), HER2-5922-2 (5′-GGGAAAGAGGAGGGGGTGAGAGGAGTGGGG-3′; SEQ ID NO: 7), and HER2-40118 (5′-GGGGGAAATAGGGAGGGTGGGG-3′; SEQ ID NO: 8). HER2-1 hybridizes to SEQ ID NO:1, under physiological conditions. HER2-205 hybridizes to SEQ ID NO:2, under physiological conditions. HER2-5922-2 hybridizes to SEQ ID NO: 5, under physiological conditions. HER2-40118 hybridizes to SEQ ID NO: 6, under physiological conditions (FIGS. 1D and 4A).

As used, the term “complementary” refers to the capacity for precise pairing (also referred to as hybridization) between two nucleotides. For example, if a nucleotide at a certain position of an oligonucleotide is capable of hydrogen bonding with a nucleotide at a corresponding position of a target RNA, then the nucleotide of the oligonucleotide and the nucleotide of the target RNA are complementary to each other at that position. As understood by one of ordinary skill in the art, for complementary base pairings, adenosine-type bases (A) are complementary to thymidine-type bases (T) or uracil-type bases (U), cytosine-type bases (C) are complementary to guanosine-type bases (G), and universal bases such as 3-nitropyrrole or 5-nitroindole can hybridize to and are considered complementary to any A, C, U, or T. In some embodiments, the methods and agents of the present disclosure can include Inosine (I). Inosine has also been considered in the art to be a universal base and is considered complementary to A, C, U or T.

In some embodiments, the TFO is a nucleotide sequence that is at least 90% identical to SEQ ID NO:3, SEQ ID NO:4, SEQ ID NO: 7 or SEQ ID NO: 8. As used herein, the term “identity” or “identical” refers to sequence identity, which refers to two nucleotides being identical. In some embodiments, the TFO is a nucleotide at least 90%, 91%, 92%, 93%, 94%, 95%, 96%, 97%, 98%, 99% or 100% identical to SEQ ID NO:3, SEQ ID NO:4, SEQ ID NO: 7 or SEQ ID NO: 8.

The sequence on a HER2 gene to which a TFO is targeted is referred to as a “target sequence.” For example, a TFO that is targeted to a polypurine site is one that hybridizes to that polypurine site under physiological conditions, such as in the case of in vivo administration or treatment. “Targeted” can also refer to a TFO that specifically hybridizes to a polypurine site (partially or completely). For example, the TFO hybridizes to a sequence in the target sequence or target sequence, but does not hybridize to any other (off-target) nucleotide sequence within the cell and would not hybridize to a sequence within a cell that lacks the polypurine site, under physiological conditions. A polypurine site within an amplified cancer-promoting gene is also referred to as a targeted amplified gene locus.

In the methods of the present disclosure, TFOs are contacted with cancer cells, such as p53-depleted cells, that comprise amplified HER2 gene by a variety of approaches, such as by administering TFOs to an individual in need of a reduction in a population of p53-deficient cells that comprise amplified HER2 gene. For example, TFOs in an appropriate delivery vehicle can be administered to an individual with cancer in which cancer cells are p53 depleted and comprise amplified HER2 gene. The TFOs are contacted with such cells by any manner and under conditions that result in entry into cells in the individual. For example, TFOs can be introduced into an individual by injection, infusion, or any delivery method, such as those described further below.

In some embodiments of the present disclosure, the TFOs are administered to an individual who has been screened for a p53 mutation, has HER2-positive cancer cells, and thus has been identified as a candidate for this p53-independent TFO-treatment. Non-limiting examples of methods for identifying an individual with p53 mutations include genetic testing of the DNA found in sera or other body fluids (see Rivlin et al., Genes & Cancer 2(4):466-474, 2011, the relevant disclosures of which are herein incorporated as reference).

In some embodiments, the methods of the present disclosure are for the treatment of p53-mutated cancers. Non-limiting examples of such p53-mutated cancers include breast cancer, ovarian cancer, renal carcinoma, lung cancer, colon carcinoma, hepatocellular carcinoma, prostate cancer, bladder cancer, and pancreatic neoplasia. The methods of the present disclosure can include administering the TFOs herein to the cells of the aforementioned cancers (e.g., breast cancer cells, ovarian cancer cells, renal cell carcinoma cells, lung cancer cells, colon cancer cells, colorectal cancer cells, gastric cancer cells, and endometrial cancer cells).

### Li-Fraumeni Syndrome Application

In some embodiments, the present disclosure includes methods and compositions for the treatment of an individual with Li-Fraumeni Syndrome (LFS) or treatment of a cancer in an individual with LFS. LFS is a cancer predisposition syndrome characterized by germline mutations of p53 (Smith and Seo, Mutagenesis 17(2), 149-156, 2002). Individuals with LFS are susceptible to a broad spectrum of cancers and are susceptible to early onset of these cancers. Of the spectrum of Li-Fraumeni-associated tumors, breast cancer, sarcomas of the soft tissues and bone, acute leukemias, and brain tumors are among the most common (Nichols et al., Cancer Epidemiology and Prevention Biomarkers 10(2): 83-87, 2001, the relevant disclosures of which are herein incorporated by reference). The lifetime risk of an LFS patient to develop cancer has been estimated to be as high as 90%. Non-limiting examples of types of cancer commonly found in families with LFS include osteosarcoma (bone cancer), soft-tissue sarcoma, acute leukemia, breast cancer, brain cancer, adrenal cortical tumors, and acute leukemia.

In some embodiments, an individual is screened for HER2 gene amplification before administration of the TFO. Methods for detection of gene amplification are known in the art. Non limiting examples of these methods include conventional cytogenetics, Southern blotting, quantitative PCR, fluorescence in situ hybridization (FISH), comparative genomic hybridization (CGH), and microarray technology.

Due to the limited toxicity associated with the described TFOs, in alternative embodiments, a TFO targeted to at least one sequence in HER2 gene can be administered to an individual diagnosed with cancer to induce p53 independent apoptosis in p53-depleted cells comprising an amplified HER2 gene prior to or without screening for HER2 gene amplification.

### Chemical Modifications to TFOs

In some embodiments, the TFOs have backbone modifications. Unmodified purine TFOs bind well under physiologic conditions, but binding efficiency can sometimes be inhibited at physiologic K+ conditions. Backbone modifications can augment the binding efficiency of such TFOs. Various modifications for purine TFOs are disclosed in Knauert and Glazer, Human Molecular Genetics 10(20): 2243-2251, 2001, the relevant disclosures of which are herein incorporated by reference. Non-limiting examples of backbone modifications to the TFOs include phosphorothioates, phosphorodithioates, methylphosphonates, phosphoramidates, boranophosphate oligos, polyamides, methylene (methylimino) linkages, morpholino oligos, and combinations thereof. TFOs with polyamide backbone modifications bind to the minor groove of the DNA duplex, rather than the major groove.

### Combination Therapies

Disclosed herein are methods of administering TFOs in sufficient quantity to induce p53-independent apoptosis in p53-depleted cells comprising an amplified HER2 gene. In some embodiments, one type of TFO (e.g., either HER2-1 or HER2-205) is administered. In some embodiments, TFOs of more than one type are administered (e.g., a mixture of TFOs, a mixture of HER2-1 and HER2-205, etc.). Herein, reference to “administering TFOs” can also refer to the administration of TFOs of more than one type.

In some embodiments, the one type of TFO is administered in combination with at least one non-TFO (e.g., a non-TFO anticancer agent). In some embodiments, more than one type of TFO is administered with at least one non-TFO (e.g., a non-TFO anticancer agent). An anticancer agent that is not a TFO can be, for example, a protein, a nucleic acid, a small molecule, or a drug for the treatment of cancer. This anticancer agent can have any anticancer effect on the population of cells that it is administered to including, but not limited to, a cytotoxic, apoptotic, anti-mitotic anti-angiogenesis or inhibition of metastasis effect. This anticancer agent can also affect DNA damage response (e.g., a DNA repair inhibitor). In some embodiments, the second anticancer agent is a drug directed against overexpressed protein products.

Anticancer agents include, for example, antimetabolites, inhibitors of topoisomerase I and II, alkylating agents and microtubule inhibitors (e.g., taxol). Non-limiting examples of anticancer agents include adriamycin aldesleukin; alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemcitabine, gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); uracil mustard; valrubicin; valtorcitabine (monoval LDC); vinblastine; vinorelbine; zoledronate; and mixtures thereof, among others (see U.S. Pat. No. 9,643,922, the relevant disclosures of which are herein incorporated by reference).

Non-limiting examples of anticancer agents include oestrogen receptor modulators, androgen receptor modulators, retinoid receptor modulators, cytotoxic agents, antiproliferative agents, prenyl-protein transferase inhibitors, HMG-CoA reductase inhibitors, reverse transcriptase inhibitors and further angiogenesis inhibitors.

Non-limiting examples of retinoid receptor modulators include bexarotene, tretinoin, 13-cis-retinoic acid, 9-cis-retinoic acid, .alpha.-difluoromethylornithine, ILX23-7553, trans-N-(4′-hydroxyphenyl)retinamide and N-4-carboxyphenylretinamide (see U.S. Pat. No. 10,093,623, the relevant disclosures of which are herein incorporated by reference).

Non-limiting examples of cytotoxic agents include tirapazimine, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosylate, trofosfamide, nimustine, dibrospidium chloride, pumitepa, lobaplatin, satraplatin, profiromycin, cisplatin, irofulven, dexifosfamide, cis-aminedichloro(2-methylpyridine)platinum, benzylguanine, glufosfamide, GPX100, (trans,trans,trans)bis-mu-(hexane-1,6-diamine)-mu-[diamineplatinum(II)]bis[diamine(chloro)platinum(II)]tetrachloride, diarisidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755 and 4-demethoxy-3-deamino-3-aziridinyl-4-methylsulfonyldaunorubicin (see WO 00/50032, the relevant disclosures of which are herein incorporated by reference).

Non-limiting examples of antiproliferative agents include antisense RNA and DNA oligonucleotides such as G3139, ODN698, RVASKRAS, GEM231 and INX3001 and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydrobenzofuryl)sulfonyl]-N′-(3,4-dichlorophenyl)urea, N6-[4-deoxy-4-[N2-[2(E),4(E)-tetradecadienoyl]-glycylamino]-L-glycero-B-L-mannoheptopyranosyl]adenine, aplidine, ecteinascidin, troxacitabine, 4-[2-amino-4-oxo-4,6,7,8-tetrahydro-3H-pyrimidino[5,4-b]-1,4-thiazin-6-yl-(S)ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetr-acyclo(7.4.1.0.0)tetradeca-2,4,6-trien-9-ylacetic acid ester, swainsonine, lometrexol, dexrazoxane, methioninase, 2′-cyano-2′-deoxy-N4-palmitoyl-1-B-D-arabinofuranosyl cytosine and 3-aminopyridine-2-carboxaldehyde thiosemicarbazone. “Antiproliferative agents” also include monoclonal antibodies to growth factors other than those listed under “angiogenesis inhibitors”, such as trastuzumab (for examples, see U.S. Pat. No. 6,069,134, the relevant disclosures of which are herein incorporated by reference).

The first drugs directed against overexpressed protein products were major breakthroughs in cancer therapeutics. For example, trastuzumab (HERCEPTIN®) targets the HER2 receptor tyrosine kinase, which is overexpressed in about 25% of breast tumors due to gene amplification¹⁰. Trastuzumab works, at least in part, by disrupting HER2 signaling, which results in cell cycle arrest and suppression of cell growth and proliferation¹¹. While trastuzumab has proven to be effective in prolonging the survival of HER2-positive breast cancer patients, primary and acquired drug resistance limits overall success rates. Similar problems hamper the long-term efficacy of other cancer drugs, including the tyrosine kinase inhibitors gefitinib (IRESSA®) and erlotinib (TARCEVA®), which target EGFR gene amplification in breast, colorectal, and lung cancer^{12,13}.

### Methods of Administering TFOs

The TFOs of the present disclosure may be administered to an individual by any route or in any delivery vehicle.

In some embodiments, the TFOs are administered in a delivery vehicle (e.g., lipid-based nanoparticles). The TFOs can be conjugated to the lipid-based nanoparticles. Alternatively, the TFOs can be encapsulated in the lipid-based nanoparticles. One example of lipid-based nanoparticles is lipid nanoparticles that contain a solid lipid core matrix with the ability to solubilize lipophilic molecules. The term “solid” refers to a nanoparticle that is solid at room temperature and atmospheric pressure. The lipid nanoparticles can have a nanostructure core (solid or hollow) and a lipid layer. The diameter of the core can be less than or equal to about 500 nm, less than or equal to about 250 nm, less than or equal to about 100 nm, less than or equal to about 75 nm, less than or equal to about 50 nm, less than or equal to about 40 nm, less than or equal to about 35 nm, less than or equal to about 30 nm, less than or equal to about 25 nm, less than or equal to about 20 nm, less than or equal to about 15 nm, or less than or equal to about 5 nm. In some embodiments, the core is less than 1000 nm. In some embodiments, the core is 1 nm, 2 nm, 3 nm, 4 nm, 5 nm, 6 nm, 7 nm, 8 nm, 9 nm, 10 nm, 20 nm, 30 nm, 40 nm 50 nm, 60 nm, 70 nm, 80 nm, 90 nm, 100 nm, 120 nm, 140 nm, 160 nm, 180 nm, 200 nm, 220 nm, 240 nm, 260 nm, 280 nm, 300 nm, 320 nm, 340 nm, 360 nm, 380 nm, 400 nm, 420 nm, 440 nm, 460 nm, 480 nm, or 500 nm in diameter.

The lipid nanoparticle can be a solid lipid nanoparticle or a polymeric nanoparticle. Methods for making solid liquid nanoparticles are well-established in the art (see, for example, Gasco, M. R., Nanoparticelle Lipidiche Solide Quali Sistemi Terapeutici Colloidali, NCF nr. 7: 71-73, 1996; Kozariara et al., Pharmaceutical Research, 20(11): 1772, 2003; and Lockman et al., Journal of Controlled Release, 93:271-282, 2003, the relevant disclosures of which are herein incorporated by reference).

In a polymeric lipid nanoparticle the polymer can be any ionic or ionizable polymer or copolymer known to those of skill in the art including polymers and copolymers of, for example, polyglycine, polyethylene glycol, heparin, hydroxypropylmethylcellulose, hydroxyethylcellulose, hydroxypropylcellulose, polyvinylpyrrolidone, polyvinyl alcohol, poly-beta amino esters (PBAEs), methacrylic acid copolymers, ethyl acrylate-methyl methacrylate copolymers, and mixtures thereof. In some embodiments, the first functionalized polymer can be poly(glycolic acid), poly(lactic acid) (PLA), or copolymers thereof, such as poly(D,L-lactide-co-glycolide), or mixtures thereof.

In some embodiments, the TFOs are delivered using poly(lactic-co-glycolic acid) (PLGA) nanoparticles or PLA nanoparticles. In some embodiments, the PLGA nanoparticles or PLA nanoparticles are loaded with the TFOs of the present disclosure. In some embodiments the nanoparticles include an agent conjugated to their surface, such as polyethylene glycol (PEG) and hyperbranched polyglycerols (HPG).

In some embodiments, the lipid nanoparticles include cationic lipids or anionic lipids. Alternatively, the lipid nanoparticles can include neutral lipids. Non-limiting examples of cationic lipids include 3β-(N—(N′,N′-dimethylaminoethane)-carbamoyl)cholesterol hydrochloride (DC-Chol); 1,2-dioleoyl-3-trimethylammonium-propane (DOTAP); 1,2-dioleoyl-3-dimethylammonium-propane (DODAP); dimethyldioctadecylammonium bromide salt (DDAB); 1,2-dilauroyl-sn-glycero-3-ethylphosphocholine chloride (DL-EPC); N-(1-(2,3-dioleyloyx) propyl)-N—N—N-trimethyl ammonium chloride (DOTMA); N-(1-(2,3-dioleyloyx) propyl)-N—N—N-dimethyl ammonium chloride (DODMA); N,N-dioctadecyl-N,N-dimethylammonium chloride (DODAC); N-(1-(2,3-dioleyloxy)propyl)-N-2-(sperminecarboxamido)ethyl)-N,N-dimethylammonium trifluoracetate (DOSPA); 1,2-dimyristyloxypropyl-3-dimethylhydroxyethyl ammonium bromide (DMRIE); dioctadecylamidoglycylspermine (DOGS); neutral lipids conjugated to cationic modifying groups; and combinations thereof. Non-limiting examples of anionic lipids include fatty acids such as oleic, linoleic, and linolenic acids; cholesteryl hemisuccinate; 1,2-di-O-tetradecyl-sn-glycero-3-phospho-(1′-rac-glycerol) (Diether PG); 1,2-dimyristoyl-sn-glycero-3-phospho-(1′-rac-glycerol) (sodium salt); 1,2-dimyristoyl-sn-glycero-3-phospho-L-serine (sodium salt); 1-hexadecanoyl,2-(9Z,12Z)-octadecadienoyl-sn-glycero-3-phosphate; 1,2-dioleoyl-sn-glycero-3-(phospho-rac-(1-glycerol)) (DOPG); dioleoylphosphatidic acid (DOPA); and 1,2-dioleoyl-sn-glycero-3-phospho-L-serine (DOPS); anionic modifying groups conjugated to neutral lipids; and combinations thereof. Non-limiting examples of neutral lipids include phosphatidylcholine (PC), phosphatidylethanolamine, ceramide, cerebrosides, sphingomyelin, cephalin, cholesterol, diacylglycerols, glycosylated diacylglycerols, prenols, lysosomal PLA2 substrates, and N-acylglycines. Additional examples of lipids and lipid components can be found in U.S. Pat. No. 9,833,416.

The lipid nanoparticles can comprise surfactants and/or emulsifiers. Non-limitng examples of surfactants include phospholipids, phosphatidylcholines, TWEENs, Soy lecithin, egg lecithin (Lipoid E 80), phosphatidylcholine, poloxamer 188, 182, and 407, poloxamine 908, Tyloxapol, polysorbate 20, 60, and 80, sodium cholate, sodium glycocholate, taurocholic acid sodium salt, taurodeoxycholic acid sodium salt, butanol, butyric acid, dioctyl sodium sulfosuccinate, and monooctylphosphoric acid sodium. Non-limiting examples of emulsifiers include cationic phospholipid or non-ionic surfactant. Examples of cationic surfactant include, but are not limited to, 1,2-dimyristoyl-3-trimethylammonium-propane (DMTAP), 1,2-dipalmitoyl-3-trimethylammonium-propane (DPTAP), 1,2-distearoyl-3-trimethylammonium-propane (DSTAP), 1,2-dioleoyl-3-trimethylammonium-propane (DOTAP), 1,2-dimyristoyl-3-dimethylammonium-propane (DMDAP), 1,2-dipalmitoyl-3-dimethylammonium-propane (DPDAP), 1,2-dilauroyl-3-dimethylammonium-propane (DLDAP), 1,2-distearoyl-3-dimethylammonium-propane (DSDAP), dimethyldioctadecylammonium chloride (DDAB), N-[1-(1,2-dioleoyloxy)propyl]-N,N,N-trimethylammonium chloride (DOTMA) and 1,2-dioleoyl-3-ethylphosphocholine (DOEPC). Examples of non-ionic surfactants include, but are not limited to poloxamers, sorbitan esters (Span), polyoxyethylene-sorbitan fatty acid esters (Tween) and polyoxyethylene ethers (Brij).

In certain embodiments, the lipid comprises one or more of: a) cationic or anionic lipids or surfactants; b) neutral lipids or surfactants; c) cholesterol; and d) PEGylated lipids or surfactants.

Other non-limiting examples of lipid-based nanoparticles include liposomes, bolaamphiphiles, nanostructured lipid carriers (NLC), and monolayer membrane structures (e.g., archaeosomes and micelles). Methods of encapsulating agents in lipid nanoparticles are disclosed in Puri et al. Critical Reviews in Therapeutic Drug Carrier Systems 26(6):523-580, 2009, the relevant disclosures of which are herein incorporated by reference.

In some embodiments, the TFOs are conjugated to cholesterol to enhance delivery into cells. In some embodiments, the TFO are administered absent of a transport peptide or cell-penetrating peptide (CPP). In some embodiments, the TFOs are administered with a peptide, e.g., cell-penetrating peptides (CPPs), primary amphipathic peptides, such as MPG or Pep-1.

The administration of the TFOs can be directly to tissue in an individual. In some embodiments, the TFOs are delivered systemically. In some embodiments, the TFOs are delivered locally or intratumorally. In some embodiments, the TFOs are administered as an injection. As used, an injection can use different delivery routes. In some embodiments, the TFOs are administered intravenously or intraperitoneally. In some embodiments, the TFOs are administered intravenously, intradermally, intraarterially, intralesionally, intratumorally, intracranially, intraarticularly, intraprostaticaly, intrapleurally, intratracheally, intranasally, intravitreally, intravaginally, intrarectally, topically, intramuscularly, intraperitoneally, subcutaneously, subconjunctival, intravesicularlly, mucosally, intrapericardially, intraumbilically, intraocularally, orally, locally, inhalation (e.g., aerosol inhalation), injection, infusion, continuous infusion, localized perfusion bathing target cells directly, via a catheter, via a lavage, in creams, in lipid compositions (e.g., liposomes, lipid nanoparticles, etc.), or by other method or any combination of the forgoing as would be known to one of ordinary skill in the art (see, for example, Remington's Pharmaceutical Sciences (1990), incorporated herein by reference).

In some embodiments, the TFOs are administered as a composition having a pharmaceutically acceptable carrier. In some embodiments, the pharmaceutically acceptable carrier comprises water or saline.

The term “pharmaceutically-acceptable carrier” refers to one or more compatible solid or liquid filler, diluents or encapsulating substances which are suitable for administration to a human or other subject contemplated by the disclosure. “Pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers (e.g., antioxidants), gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated.

The TFOs can be administered once, or alternatively they may be administered in a plurality of administrations. If administered multiple times, the compounds may be administered via different routes. For example, the first (or the first few) administrations may be made directly into the affected tissue while later administrations may be systemic.

In some aspects of the present disclosure, the term “individual” refers to a mammal. In some embodiments, individual refers to a human. Alternatively, individual can refer to a mammal, wherein the mammal is selected from a group including but not limited to non-human primates, cows, horses, pigs, sheep, goats, dogs, cats, rabbits, ferrets, and rodents. In some embodiments, the term “individual” is used to refer to a model of cancer. Non-limiting examples of models of cancer include p53-knockout mice, BALB/c mice injected with MDA-MB-453 cells, SKBR3 cells, BT474 cells, PEO1 cells, or SKOV3 cells, and LFS mouse models (engineered to express mutant p53).

As used herein, the term “sufficient quantity” refers to a “therapeutically effective amount” or “effective amount” that elicits a biological or medicinal response in a tissue, system, animal, individual or human that is being sought by a researcher, veterinarian, medical doctor or other clinician. In this case, the response would be a reduction (partial or total/complete) in the number of p53-depleted cancer cells comprising a HER2 gene by apoptosis. The appropriate response can be determined in vitro by trypsinization and cell counting (using methods established in the art). In vivo, the appropriate response from a therapeutically effective amount can be determined by measuring or visualizing (e.g., imaging) tumor size.

In some embodiments, the TFOs are administered to an individual in a dose of approximately 20 mg/kg. In some embodiments, the TFOs are administered to an individual in a dose of 5 mg/kg, 7 mg/kg, 9 mg/kg, 10 mg/kg, 12 mg/kg, 14 mg/kg, 16 mg/kg, 17 mg/kg, 18 mg/kg, 19 mg/kg, 20 mg/kg, 21 mg/kg, 22 mg/kg, 25 mg/kg, or 30 mg/kg. These disclosed amounts can be increased or decreased by one of ordinary skill in order to personalize a chemotherapeutic treatment plan based on an individual's stage of cancer, the size of tumor, the presence or absence of an adjuvant chemotherapeutic agent, etc.

### TFO Use for Assays

In alternate embodiments, the method is an assay in which TFOs are contacted in vitro with p53-depleted cancer cells comprising a HER2 gene. As used, the term “contacting” refers to the use of TFOs in in vitro assays. The contacting can be through the transfection of cells with the TFOs in vitro. In some embodiments, the cells are cancer cells. In some embodiments, the cancer cells are contacted with TFOs under conditions under which the TFOs enter the p53-depleted cancer cells in sufficient quantity to induce apoptosis under the conditions of the in vitro assay.

Methods of transfection are well established in the arts and include chemical, biological, and physical methods. Chemical methods include, but are not limited to, calcium phosphate transfection, cationic polymer transfection (e.g., polyethylenimine), lipofection, Oligofectamine™, DharmaFECT-1™, FUGENE®, and DEAE-Dextran-mediated transfection. Other methods of transfection include, but are not limited to, electroporation, microinjection, sonoporation, cell squeezing, impalefection, optical transfection, protoplast fusion, Magnetofection™, and particle bombardment.

Non-limiting examples of cells that can be contacted by any of the TFOs described herein for in vitro assays, include carcinoma cells, lung cancer cells, colon cancer cells, human colon carcinoma cells, ovarian cancer cells, breast cancer cells, colorectal cancer, gastric cancer cells, and endometrial cancer cells. Additional non-limiting examples of cells that can be contacted by any of the TFOs described herein for in vitro assays, include MDA-MB-453 cells, SKBR3 cells, BT474 cells, PEO1 cells, or SKOV3 cells.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference.

### Additional Embodiments

1. A method of reducing, in a population of cells, the number of p53-depleted cancer cells in which a HER2 gene is amplified, the method comprising contacting p53-depleted cancer cells with triplex forming oligonucleotides (TFOs) targeted to a polypurine target site in the amplified-HER2 gene, under conditions under which the TFOs enter the p53-depleted cancer cells in sufficient quantity to induce apoptosis.
2. The method of paragraph 1, wherein the p53-depleted cells are mammalian cells.
3. The method of paragraph 1, wherein the p53-depleted cells are human cells.
4. The method of paragraph 1, wherein the polypurine target site is/comprises SEQ ID NO: 1, SEQ ID NO: 2, SEQ ID NO: 5 or SEQ ID NO: 6.
5. The method of paragraph 1, wherein the TFOs are at least 13 nucleotides in length.
6. The method of paragraph 1, wherein the TFOs are at least 22 nucleotides in length.
7. The method of paragraph 5 or 6, wherein at least 13 of the nucleotides hybridize to SEQ ID NO: 1, SEQ ID NO: 2, SEQ ID NO: 5 or SEQ ID NO: 6.
8. The method of paragraph 1, wherein the TFOs comprise a nucleotide sequence at least 90% identical to SEQ ID NO: 3 and/or a nucleotide sequence at least 90% identical to SEQ ID NO: 4 and/or a nucleotide sequence at least 90% identical to SEQ ID NO: 7 and/or a nucleotide at least 90% identical to SEQ ID NO: 8.
9. The method of any one of paragraphs 1-8, wherein the triplex forming oligonucleotides (TFOs) are in a delivery vehicle or are conjugated to a delivery vehicle.
10. The method of paragraph 9, wherein the delivery vehicle is lipid nanoparticles.
11. The method of any one of paragraphs 1-10, wherein the TFOs have backbone modifications.
12. The method of paragraph 11, wherein the backbone modifications include phosphorothioates, phosphorodithioates, methylphosphonates, phosphoramidates, boranophosphate oligos, polyamides, methylene(methylimino) linkages, morpholino oligos, or some combination thereof.
13. The method of any one of paragraphs 1-12, wherein the p53-depleted cancer cells are renal cell carcinoma cells, lung cancer cells, colon cancer cells, colon carcinoma cells, ovarian cancer cells, breast cancer cells, colorectal cancer cells, gastric cancer cells, and/or endometrial cancer cells.
14. A method of reducing, in a population of cells, the number of p53-mutated cancer cells in which a HER2 gene is amplified, the method comprising contacting p53-mutated cancer cells with triplex forming oligonucleotides (TFOs) targeted to a polypurine site in the amplified-HER2 gene, under conditions under which the TFOs enter the p53-mutated cancer cells in sufficient quantity to induce apoptosis.
15. The method of paragraph 14, wherein the p53-mutated cells are mammalian cells.
16. The method of paragraph 14, wherein the p53-mutated cells are human cells.
17. The method of paragraph 14, wherein the polypurine target site is SEQ ID NO: 1, SEQ ID NO: 2, SEQ ID NO: 5 or SEQ ID NO: 6.
18. The method of paragraph 14, wherein the TFOs are at least 13 nucleotides in length.
19. The method of paragraph 14, wherein the TFOs are at least 22 nucleotides in length.
20. The method of paragraph 18 or 19, wherein at least 13 of the nucleotides hybridize to SEQ ID NO: 1, SEQ ID NO: 2, SEQ ID NO: 5 or SEQ ID NO: 6.
21. The method of paragraph 14, wherein the TFOs comprise a nucleotide sequence at least 90% identical to SEQ ID NO: 3, and/or a nucleotide sequence at least 90% identical to SEQ ID NO: 4, and/or a nucleotide sequence at least 90% identical to SEQ ID NO: 7 and/or a nucleotide at least 90% identical to SEQ ID NO: 8.
22. The method of any one of paragraphs 14-21, wherein the TFOs are in a delivery vehicle or are conjugated to a delivery vehicle.
23. The method of paragraph 22, wherein the delivery vehicle is lipid nanoparticles.
24. The method of any one of paragraphs 14-23, wherein the TFOs have backbone modifications.
25. The method of paragraph 24, wherein the backbone modifications include phosphorothioates, phosphorodithioates, methylphosphonates, phosphoramidates, boranophosphate oligos, polyamides, methylene(methylimino) linkages, morpholino oligos, or some combination thereof.
26. The method of any one of paragraphs 14-25, wherein the p53-mutated cancer cells are renal cell carcinoma cells, lung cancer cells, colon cancer cells, colon carcinoma cells, ovarian cancer cells, breast cancer cells, colorectal cancer cells, gastric cancer cells, and/or endometrial cancer cells.
27. A method of treating cancer in an individual with Li-Fraumeni syndrome, the method comprising administering to the individual TFOs targeted to a polypurine target site in an amplified-HER2 gene, under conditions under which the TFOs enter p53-depleted cancer cells in sufficient quantity to induce apoptosis.
28. The method of paragraph 27, wherein the polypurine target site is SEQ ID NO: 1, SEQ ID NO: 2, SEQ ID NO: 5 or SEQ ID NO: 6.
29. The method of paragraph 27, wherein the TFOs are at least 13 nucleotides in length.
30. The method of paragraph 27, wherein the TFOs are at least 22 nucleotides in length.
31. The method of paragraph 29 or 30, wherein at least 13 of the nucleotides hybridize to SEQ ID NO: 1, SEQ ID NO: 2, SEQ ID NO: 5 or SEQ ID NO: 6.
32. The method of paragraph 27, wherein the TFOs comprise a nucleotide sequence at least 90% identical to SEQ ID NO: 3, and/or a nucleotide sequence at least 90% identical to SEQ ID NO: 4, and/or a nucleotide sequence at least 90% identical to SEQ ID NO: 7, and/or a nucleotide at least 90% identical to SEQ ID NO: 8.
33. The method of any one of paragraphs 27-32, wherein the TFOs are in a delivery vehicle or are conjugated to a delivery vehicle.
34. The method of paragraph 33, wherein the delivery vehicle is lipid nanoparticles, and wherein the TFOs are encapsulated in the lipid nanoparticles.
35. The method of any one of paragraph 27-34, wherein the TFOs have backbone modifications.
36. The method of paragraph 35, wherein the backbone modifications include phosphorothioates, phosphorodithioates, methylphosphonates, phosphoramidates, boranophosphate oligos, polyamides, methylene (methylimino) linkages, morpholino oligos, or some combination thereof.
37. The method of any one of paragraphs 27-36, wherein the TFOs are administered by injection.
38. The method of paragraph 37, wherein the TFOs are administered intratumorally or intraperitoneally.
39. The method of any one of paragraphs 27-38, further comprising administering an anticancer agent that is not a TFO.
40. The method of paragraph 39, wherein the anticancer agent is a protein, a nucleic acid, a small molecule, or a drug.
41. A method of administering TFOs for the treatment of cancer, the method comprising:
(i) preparing a mixture of TFOs targeted to a polypurine target site in an amplified-HER2 gene; and
(ii) administering the mixture of TFOs to an individual, in sufficient quantity to induce p53-independent apoptosis.
   42. The method of paragraph 41, wherein the mixture of TFOs is encapsulated in lipid nanoparticles.
   43. The method of paragraph 41 or paragraph 42, wherein the polypurine target site is SEQ ID NO: 1, SEQ ID NO: 2, SEQ ID NO: 5 or SEQ ID NO: 6.
   44. The method of paragraph 41 or paragraph 42, wherein the TFOs are at least 13 nucleotides in length.
   45. The method of paragraph 41 or paragraph 42, wherein the TFOs are at least 22 nucleotides in length.
   46. The method of paragraph 44 or paragraph 45, wherein at least 13 of the nucleotides hybridize to SEQ ID NO: 1, SEQ ID NO: 2, SEQ ID NO: 5 or SEQ ID NO: 6.
   47. The method of paragraph 41 or paragraph 42, wherein the mixture of TFOs comprises a nucleotide sequence at least 90% identical to SEQ ID NO: 3, and/or a nucleotide sequence at least 90% identical to SEQ ID NO: 4, and/or a nucleotide sequence at least 90% identical to SEQ ID NO: 7 and/or a nucleotide at least 90% identical to SEQ ID NO: 8.
   48. The method of any one of paragraphs 41-47, wherein the TFOs have backbone modifications.
   49. The method of paragraph 48, wherein the backbone modifications include phosphorothioates, phosphorodithioates, methylphosphonates, phosphoramidates, boranophosphate oligos, polyamides, methylene(methylimino) linkages, morpholino oligos, or some combination thereof.
   50. The method of any one of paragraphs 41-49, wherein the mixture of TFOs is administered by injection.
   51. The method of paragraph 41-50, wherein the mixture of TFOs is administered intratumorally or intraperitoneally.
   52. The method of any one of paragraphs 41-51, further comprising administering an anticancer agent that is not a TFO.
   53. The method of paragraph 52, wherein the anticancer agent is a protein, a nucleic acid, a small molecule, or a drug.
   54. The method of any one of paragraphs 41-53, wherein the individual is a mammal.
   55. The method of paragraph 54, wherein the individual is a human.
   56. The method of any one of paragraphs 41-53, wherein the individual is a model of cancer.
   57. The method of paragraph 56, wherein the cancer is a carcinoma, a sarcoma or a melanoma with HER-2 gene amplification.
   58. The method of paragraph 56 or paragraph 57, wherein the model of cancer is selected from a group including a p53-knockout mouse, a Li-Fraumeni Syndrome mouse, a mouse with MDA-MB-453 cells, SKBR3 cells, BT474 cells, PEO1 cells, SKOV3 cells, and p53-knockout mouse.
   59. A composition, comprising:
(i) TFOs targeted to a polypurine target site in an amplified-HER2 gene in sufficient quantity to induce p53-independent apoptosis in a p53-depleted cancer cell or a p53-mutated cancer cell; and
(ii) a pharmaceutically acceptable carrier.
   60. The composition of paragraph 59, further comprising:
(iii) lipid nanoparticles, wherein the TFOs are encapsulated in the lipid nanoparticles or wherein the TFOs are conjugated to the lipid nanoparticles.
   61. The composition of paragraph 59 or 60, wherein the polypurine target site is SEQ ID NO: 1, SEQ ID NO: 2, SEQ ID NO: 5 or SEQ ID NO: 6.
   62. The composition of paragraph 59 or 60, wherein the TFOs are at least 13 nucleotides in length.
   63. The composition of paragraph 59 or 60, wherein the TFOs are at least 22 nucleotides in length.
   64. The composition of paragraphs 62 or 63, wherein at least 13 of the nucleotides hybridize to SEQ ID NO: 1, SEQ ID NO: 2, SEQ ID NO: 5 or SEQ ID NO: 6.
   65. The composition of paragraph 59 or 60, wherein the TFOs comprise a nucleotide sequence at least 90% identical to SEQ ID NO: 3, and/or a nucleotide sequence at least 90% identical to SEQ ID NO: 4, and/or a nucleotide sequence at least 90% identical to SEQ ID NO: 7 and/or a nucleotide at least 90% identical to SEQ ID NO: 8.
   66. The composition of any one of paragraphs 59-65, wherein the TFOs have backbone modifications.
   67. The composition of paragraph 66, wherein the backbone modifications include phosphorothioates, phosphorodithioates, methylphosphonates, phosphoramidates, boranophosphate oligos, polyamides, methylene(methylimino) linkages, morpholino oligos, or some combination thereof.
   68. The composition of any one of paragraphs 59-67, wherein the pharmaceutically acceptable carrier comprises water or saline.

### Examples

Advancements in DNA sequencing technology have not only revealed commonly mutated and deleted genes across cancer types but also enabled identification of amplified cancer-promoting genes⁸. These amplified genes include epigenetic regulators, cell cycle-associated genes, and genes linked to signaling pathways, such as the EGFR and HER2 genes⁹. The first drugs directed against the overexpressed protein products encoded by these genes were major breakthroughs in cancer therapeutics. For example, trastuzumab targets the HER2 receptor tyrosine kinase, which is overexpressed in ˜25% of breast tumors due to gene amplification¹⁰. Trastuzumab works, at least in part, by disrupting HER2 signaling, resulting in cell cycle arrest and suppression of cell growth and proliferation¹¹. Trastuzumab has proven to be effective in prolonging the survival of HER2-positive breast cancer patients, but primary and acquired drug resistance limits overall success rates. Similar problems hamper the long-term efficacy of other cancer drugs, including the tyrosine kinase inhibitors gefitinib and erlotinib, which target EGFR gene amplification in breast, colorectal and lung cancer^{12,13}.

### Materials and Methods

Expressly incorporated by reference herein are the materials and methods described in the section “Materials and Methods” of U.S. application Ser. No. 16/683,205 and in the section “Materials and Methods” of U.S. Provisional Application No. 62/767,279.

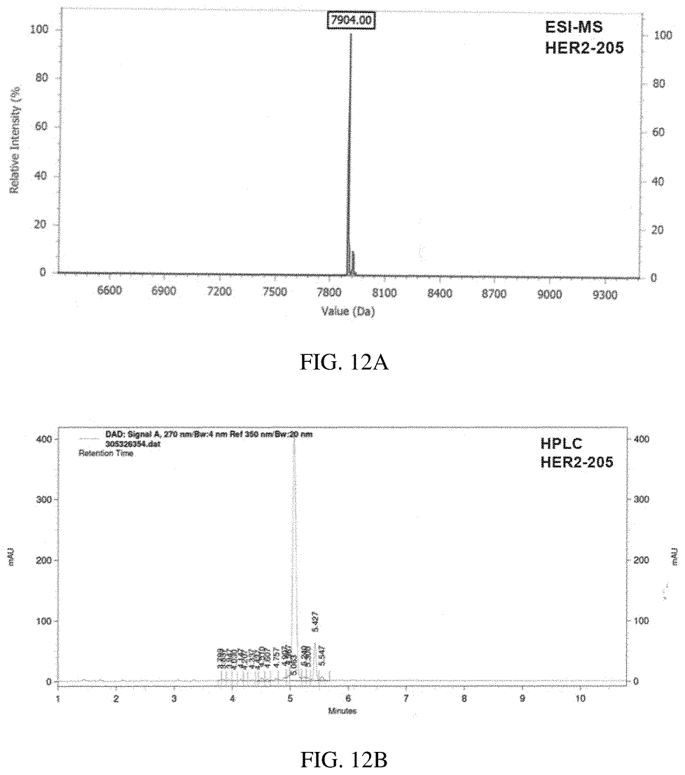

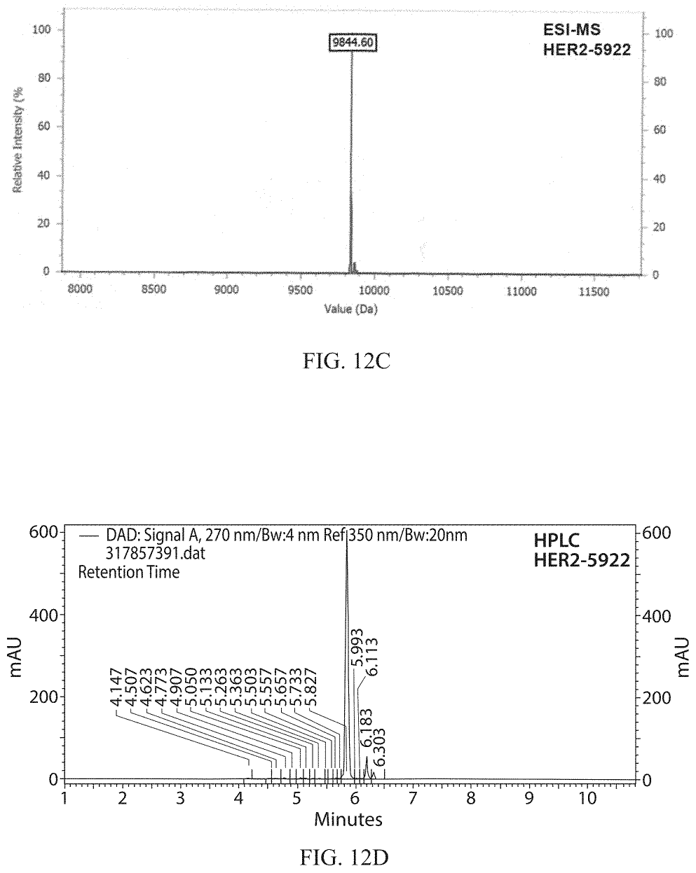

Oligonucleotides. Oligonucleotides were synthesized by IDT with a 3′-amino modifier and purified by reverse-phase HPLC and analyzed by ESI-MS (FIGS. 12A-12D). The TFO, HER2-1 was designed to bind to the HER2 promoter with the sequence 5′-GGGAGGAGGAGGTGGAGGAGGAAGAGGA-3′ (SEQ ID NO: 3). HER2-205 was synthesized with the sequence 5′-GAGGAGGAGTGGGAGAATGGGGGG-3′ (SEQ ID NO: 4) and has been designed to bind to a polypurine sequence in the coding region of the HER2 gene. The TFO, HER2-5922 was designed to bind to a region of intron 2, with the sequence 5′ GGGAAAGAGGAGGGGGTGAGAGGAGTGGGG 3′ (SEQ ID NO: 7). HER2-40118 was synthesized with the sequence, 5′ GGGGGAAATAGGGAGGGTGGGG 3′ (SEQ ID NO: 8) and has been designed to bind to a polypurine sequence in intron 19 of the HER2 gene. The control mixed-sequence oligonucleotide MIX24 has the following sequence: 5′-AGTCAGTCAGTCAGT CAGTCAGTC-3′ (SEQ ID NO: 9). Labeled oligonucleotide was synthesized with 5′-TAMRA modifications.
Cell Lines and Transfections. Human breast cancer cell lines were obtained from ATCC and routinely tested for mycoplasma. The human cell lines, MDA-MB-453, SKBR3, and BT474 are HER2-amplified breast cancer cell lines. BT20 and MCF7 cells are non-amplified breast cancer cell lines. MCF-10A is a non-tumorigenic breast epithelial cell line. PEO1 and SKOV3 are human ovarian cancer cell lines with HER2 gene amplification.

Cells were seeded in six-well plates at a density of 2-4×10⁵ cells per well the day before transfection. Cells were transfected with 2 μg (˜100 nM) of HER2-targeted TFO or MIX24 using Oligofectamine (Invitrogen) or Dharmafect-1 (Dharmacon) transfection reagent. Transfection was performed as per manufacturer's instructions. siRNA directed against p53, XPD and non-target controls (ON-Target plus SMARTpool reagents; Dharmacon) were transfected into BT474 cells using Dharmafect-1 transfection reagent (Dharmacon) according to the manufacturer's instructions. Western blot analysis was used to confirm knockdown of protein.

Metaphase Chromosome Spreads. Cells were transfected with 2 μg of TAMRA labeled HER2-205. Twenty-four hours post-transfection, cells were treated for 5 h with Colcemid (0.1 μg/μ1). Cells were then collected and washed once with PBS. To the cell pellet, a 75 mM KCl solution was added for 20 minutes at 37° C. Cell pellets were then resuspended in Carnoy's fixative solution (75% methanol, 25% acetic acid). Following 10 minutes incubation at room temperature, the cells were pelleted and resuspended in an additional 500 μl of Carnoy's fixative solution (3:1 methanol:acetic acid). Cells were dropped from a height onto glass slides and mounting medium with DAPI (Prolong Gold antifade reagent, Invitrogen) was added to each slide. A FITC labeled satellite probe specific for human chromosome 17 (Cytocell) was used to detect gene-specific triplex formation. Pictures were taken of 50-60 metaphase spreads using an Axiovert 200 microscope (Carl Zeiss Micro Imaging, Inc.).
Western blotting. Whole cell lysates were prepared from floating and adherent cells using RIPA or AZ lysis buffer according to standard protocols. Total protein (30-50 m per sample) was resolved by SDS-PAGE. Proteins were detected by a standard immunoblot protocol using the following primary antibodies: cleaved PARP, cleaved caspase 3, γH2AX, XPD, p53, HER2, pHER2 (Y11221/1222), HER3, pHER3 (Y1289), HER4, pHER4 (Y1284), EGFR, pEGFR (Y1068), Chk1, pChk1(S345), pChk2 (T68), and Chk2 (Cell Signaling Technology); pH2AX (tyrosine 142; EMD Millipore); tubulin (clone B-512; Sigma), γH2AX (Santa Cruz Biotechnology) and GAPDH-HRP (Proteintech). Each experiment was repeated with independent sample preparation a minimum of three times, and representative western blots are shown.
Apoptosis analysis. Cells (2-4×10⁵) were seeded in six-well plates 24 h prior to treatment with MIX24, HER2-1 or HER2-205 (2 μg). Post-treatment analysis was performed using the Annexin V-FITC/PI apoptosis detection kit (BD Pharmingen) according to the manufacturer's protocol. Apoptotic frequency was calculated as the combined percentage of early and late apoptotic cells. Data analysis was performed using FlowJo software.
Immunofluorescence. Cells were seeded onto UV-irradiated coverslips and were treated for 24 h with HER2-205, MIX24, or a mock transfection. Cells were processed 24 h post-transfection, fixed with 4% formaldehyde and then incubated with ice-cold 100% methanol for 20 minutes followed by a methanol and acetone solution (1:1) for 20 minutes each at −20° C. After washing with PBS, cells were blocked with blocking buffer (4% BSA, 0.2% Triton X-100 in PBS) for 30 minutes and then incubated overnight with the following primary antibodies: γH2AX (1:500; Cell Signaling or Millipore) and 53BP1 (1:100; Santa Cruz) in blocking buffer at 4° C. After three washes, cells were incubated with secondary antibodies Alexa 488 F(ab′)2 fragment goat anti-rabbit IgG or Alexa 568 F(ab′)2 fragment goat anti-mouse IgG (1:1000; Molecular Probes) for 1 h at room temperature. Cells were then mounted on microscope glass slides with anti-fade mounting media containing DAPI (Life Technologies), and pictures were taken with a Leica SP5 microscope. Immunofluorescence experiments were repeated for validation.
Comet Assay. Neutral comet assays were performed 24 h post TFO-transfection as per the manufacturer's instructions (Trevigen) with the adjustment of 3.5×10⁵ cells/ml for each single cell suspension and 30 minutes electrophoresis. Comets were visualized using an Axiovert 200 microscope and analyzed with Autocomet or Comet Score 2.0 software. Approximately 100-200 comets were analyzed per experiment. Experiments were performed in triplicate and results were expressed as mean tail moment and standard error of the mean.
Mouse Models. All mice were maintained at Yale School of Medicine in accordance with guidelines of the Animal Care and Use Committee of Yale University and conformed to the recommendations in the Guide for the Care and Use of Laboratory Animals (Institute of Laboratory Animal Resources, National Research Council, National Academy of Sciences).

Six to seven-week old female BALB/c athymic, ovariectomized nude mice (Harlan Sprague-Dawley) were implanted with 0.72 mg, 60-day release 17β-estradiol pellets (Innovative Research). The following day 2.5×10⁷ BT474 cells suspended in 100 μl equal volume of media and Matrigel Basement Membrane Matrix (BD Bioscience) were injected subcutaneously in the right flank of each mouse. Mice bearing a tumor of ˜100 mm³ in volume were randomly divided into four treatment groups: vehicle (PBS), n=8; mixed-sequence oligonucleotide, MIX24, n=8; HER2-targeted TFO, HER2-205, n=5; and trastuzumab (Herceptin), n=8. Mice were treated with 20 mg/kg body weight of MIX24, HER2-205 or trastuzumab in PBS by intraperitoneal injection (3 doses evenly administered over 7 days). Tumor volumes in each group were then monitored and mice were sacrificed when tumor volumes reached 1000 mm³. Error bars represent standard error of the mean. Tumor tripling time was calculated as the time required for tumors to increase in volume three-fold over baseline (defined as tumor volume before administration of dose on first day of treatment). Harvested tumors were fixed in 10% neutral buffered formalin and processed by Yale Pathology Tissue Services for H&E, Caspase 3, HER2, and Ki67. Images were taken at 4× magnification.

To establish an ovarian cancer model, female BALB/c athymic nude mice were injected subcutaneously in the flank with 5×10⁶ SKOV3 cells suspended in 100 μl equal volume of media and Matrigel. Mice bearing a tumor of ˜100 mm³ in volume were randomly divided into three treatment groups: vehicle (PBS), n=5; HER2-targeted TFO, HER2-205, n=5; and cisplatin, n=7. HER2-205 (20 mg/kg) and cisplatin (10 mg/kg) were administered by intraperitoneal injection (3 doses/once per week for three weeks). This dosing schedule was adapted from an established regimen that avoids cisplatin toxicity in this mouse line. Tumor volumes were monitored and tumor tripling times were calculated as described above.

To establish an orthotopic breast cancer model, four-week female BALB/c athymic nude mice (Envigo) were implanted with 0.72 mg, 60-day release 17β-estradiol pellets. The following day 2.5×10⁷ BT474 cells suspended in 100 μl equal volume of media and Matrigel Basement Membrane Matrix (BD Bioscience) were injected subcutaneously in the left and right inguinal mammary fat pad of each mouse. Mice bearing a tumor of ˜100 mm³ in volume were randomly divided into four treatment groups: untreated, n=8; mixed-sequence oligonucleotide, MIX24, n=8; HER2-targeted TFO, HER2-205, n=8; and HER2-targeted TFO, HER2-5922, n=8. Mice were treated with 2 mg PLA-HPG nanoparticles encapsulated (˜70 pmol TFO/mg) with either MIX24, HER2-205 or HER2-5922 in PBS (200 μl) by intravenous systemic administration using retro-orbital injection (7 doses evenly administered over 15 days). Tumors volumes were monitored by digital calipers and calculated using the formula V=(L×W×W)×0.523.

Gene Expression. RNA was extracted from snap-frozen cells using the RNeasy Kit (Qiagen) per the manufacturer's protocol. cDNA synthesis was carried out with 1 μg of RNA via reverse transcription reactions and the High-Capacity cDNA Reverse Transcription Kit (ThermoFisher or Applied Biosystems). cDNA (10 ng) was then combined with TaqMan Universal PCR master mix (20 μl) (Applied Biosystem) and primers specific to HER2 (HER2, Hs01001580_ml, ThermoFisher Scientific) or the internal control, β-actin (Hs99999903_ml, ThermoFisher Scientific). RT-PCR was performed in 96-well optical plates in triplicate for each sample. Briefly, reactions were performed at 50° C. for 2 minutes, followed by 95° C. for 10 minutes. Amplification of the target or control gene was carried out with 40 cycles of the two-step reaction, which included 95° C. for 15 seconds and 1 minute at 60° C. β-actin expression levels were used to normalize the difference between cDNA levels in different samples. Relative expression levels were calculated using the 2(−Delta Delta C(T)) method. Experiments were conducted at least three times and results are expressed as relative fold change and standard error of the mean.
Flow Cytometry. BT474 cells were collected 24 h following treatment with either MIX24 or HER2-205. After washing with PBS, cells were incubated with 1% paraformaldehyde for 15 minutes on ice. Cells were then fixed with cold 70% ethanol at −20° C. for 2 h or kept for up to 2 weeks until further analysis. Cells were centrifuged and rinsed with PBS, blocked with PBST buffer (1% w/v bovine serum albumin and 0.2% v/v Triton X-100 in PBS) for 15 minutes on ice, followed by another PBS rinse. Cells were first incubated with anti-phospho-ATM (S1981, EMD Millipore) in PBST at 1:100 dilution for 1 h at room temperature. Cells were rinsed with PBST and incubated with anti-rabbit IgG Fab2 Alexa 488 (Molecular Probes) at 1:100 dilution at room temperature for 1 h, and then rinsed with PBST. Acquisition of labeled cells and analysis of data was completed using a flow cytometer (FACS Calibur) and FlowJo software respectively.
Survival and Cell Viability Assays. Cell survival was assayed by visualization of monolayer growth. Briefly, cells were plated at a defined density in 6 or 12-well dishes and treated with either transfection reagent alone (mock), MIX24, or HER2-205 as previously described. Monolayers were visualized by staining cells with crystal violet 72 h post-treatment.

Cells (1.5×10⁴) were seeded in 96-well plates 18-24 hours prior to treatment with increasing concentration of either MIX24, HER2-1, HER2-5922-2, HER2-205 or HER2-40118. Cell viability was evaluated 48 h post-treatment using CellTiter-Glo® Luminescent Cell Viability Assay per manufacturer's instructions. Luminescent signals were normalized to mock treatment with transfection reagent alone. Experiments were performed in triplicate and results are expressed as percentage of viable cells and standard error of mean.

Fluorescence in situ Hybridization (FISH). HER2 and chromosome 17 probes were obtained from Cytocell. The HER2 gene (17q12) probe was labeled with fluorescent Texas Red spectrum and the CEP17 (17p11.1-q11.1) probe was tagged with FITC. PEO1 and SKOV3 cells were treated with colcemid (0.1 μg/ml) for three hours and collected by trypsinizing the monolayer. After washing the cells with PBS, cells were treated with a hypotonic solution (0.075 M KCl) at 37° C. for 20 minutes. Cells were then washed and fixed with Carnoy's fixative solution (methanol and acetic acid in 3:1 ratio). Cells were dropped on slides and fluorescent in situ hybridization was performed on the spreads as per the manufacturer's instructions. Images were obtained using a Zeiss microscope with Metafer software. A minimum of 50 cells were scored to quantify HER2 and chromosome 17 positive foci.
Chromatin immunoprecipitation assay (ChIP). Gene specific induction of DNA damage at the triplex site was evaluated using ChIP assays as previously described with some modifications²⁸. Briefly, BT474 cells (1.5×10⁶ cells) were transfected with MIX24 or HER2-205, and cells were collected 8 h post-transfection. Cell lysis was performed using SimpleChIP® Enzymatic Cell Lysis Buffers (Cell Signaling Technology) as per manufacturer's instructions. To obtain chromatin fragments ranging from 200-1000 bp nuclear pellets were incubated with micrococcal nuclease (0.25 μL) at 37° C. on a rotary shaker (600 rpm for 20 min). Digestion reactions were inhibited with EDTA (20 μL, 0.5M) and one min incubation on ice. ChIP was performed using γH2AX antibody (SantaCruz Biotech). Samples were then sonicated with Qsonica sonicator for 9 min set at 30 sec on/30 sec off cycles at 100% amplitude. After decrosslinking samples were purified using QIAquick PCR purification columns (Qiagen). The primers utilized in these studies for the HER2 coding region were forward primer: 5′ GAC AGT CGA GAC GCT CAG G-3′ (SEQ ID NO: 14) and reverse primer: 5′ GGA AAG CGC CAG TCT CTT GG-3′ (SEQ ID NO: 15). HER2 intron 2 primers are: forward primer: 5′ GCC TTGTAG CTA AGG ATC ACC-3′ (SEQ ID NO: 16) and reverse primer: 5′ CAA CCC CCA GGA CGA AAA AAG-3′ (SEQ ID NO: 17). qPCR was performed on samples using SYBR mastermix, 5 nM each of forward and reverse primers, 5 μL of ChIP or input DNA and amplified using a the Stepone plus quantitative PCR machine (Applied Biosystems). To analyze induction of non-specific DNA damage, SimpleChIP Human GAPDH Exon 1 primers (Cell Signaling Technology) were utilized. Fold change was calculated using the ΔΔC_{T} method. The relative enrichment was determined via normalization of C_{T} values against input followed by normalization to the untreated samples.
Transcription Inhibition Assay. To determine the role of transcription on TFO induced DNA damage, BT474 cells (400,000 cells) were pretreated for 20 h with the transcription inhibitor, α-amanitin (10 μg/μL) prior to transfection with TFOs. Following pre-treatment, the media was removed and cells were washed with PBS after which TFO treatment was performed as described above. Cells were collected 8 h after treatment and lysates were analyzed by western blot of cleaved caspase 3 and γH2AX.
Nanoparticle Delivery System. Dye-loaded NPs were synthesized using a single-emulsion technique as previously described^{27,29}. For PLGA NPs, 50 mg of polymer was dissolved in 1 mL of dichloromethane (DCM, J. T. Baker) overnight. After dissolving the polymer, 1,1′-dioctadecyl-3,3,3′,3′-tetramethylindodicarbocyanine, 4-chlorobenzenesulfonate salt (DiD) (Biotium) was added to the polymer solution at 0.5% wt:wt, DiD:PLGA. The polymer-dye solution was then added dropwise to a 5% solution of poly(vinyl alcohol) (PVA) and sonicated on ice using a probe sonicator for three, 10 second cycles, at 38% amplitude (Tekmar Company). The emulsion was then added to a stirring solution of 0.3% PVA for 3 hours. Following stirring and solvent evaporation, NPs were centrifuged (3×, 16100 g, 15 min) and washed in diH₂O to remove excess PVA prior to lyophilization (72 h). Dried NPs were stored at −20° C. until use. PLA-HPG DiD loaded particles were generated using the method described below.

Poly(lactic acid)-hyperbranched polyglycerol (PLA-HPG) polymer was synthesized and characterized as previously described^{27,30}. Nanoparticles (NPs) were synthesized as previously described using a double-emulsion solvent evaporation technique²⁷. Briefly, 50 mg of PLA-HPG polymer was dissolved in a mixture of 2.4 mL ethyl acetate (EtOAc, Sigma-Aldrich) and 0.6 mL of dimethyl sulfoxide (DMSO, J. T. Baker) overnight. Oligonucleotides (100 nmol, MIX24, HER2-205 or HER2-5922) in 100 μl of diH₂O were added drop-wise to the polymer solution under vortex. The water-in-oil mixture was immediately sonicated using a probe sonicator for three, 10 second cycles, at 38% amplitude (Tekmar Company). Following sonication, the first emulsion was added dropwise to 4 mL of diH₂O under vortex. The second emulsion was sonicated as above and diluted into 20 mL of diH₂O and placed on a rotary evaporator at room temperature to remove EtOAc. NPs were subsequently collected via centrifugation at 4,000 g for 30 min at 4° C. using a 100 kDa MWCO centrifugal filter (Amicon® Ultra-15, MilliporeSigma) and washed twice with 15 ml of diH₂O. Nanoparticle aliquots (2 mg) were prepared and stored at −80° C. until used.

The nanoparticles were characterized according to their surface charge, size and loading capacity. NP hydrodynamic size by dynamic light scatter and zeta potential were measured in water at room temperature using the Zetasizer Nano-ZS by Malvern according to manufacturer protocols (n=3). For loading analysis, 2 mg of NPs (n=3) were dissolved in DMSO and analyzed for total nucleic acid content using the Quant-iT™ OliGreen™ ssDNA Assay Kit (ThermoFisher Scientific) according to manufacturer protocols.

Nanoparticle Biodistribution Studies. PLA-HPG nanoparticles loaded with either DiD or TAMRA-HER2-205 were thawed on ice and resuspended in PBS (2 mg NP/200 μl) by vortex. In studies designed to evaluate NP formulations for their tumor uptake efficiency, a single dose of DiD loaded particles were administered via retro-orbital (RO) injection. TAMRA-HER2-205 encapsulated PLA-HPG NPs were utilized in the TFO biodistribution studies (4 tumors per treatment group/time point), with systemic administration of a single dose via RO injection. Mice were euthanized by CO₂ exposure and tumors and organs including the lungs, heart, liver, kidney and spleen, were harvested 12 and 24 h following treatment. Tumors and organ tissues were processed as paraformaldehyde fixed frozen sections.

Briefly, tumor tissues were transferred to 4% paraformaldehyde and allowed to shake on an orbital shaker for 24 h. Tissues were then equilibrated with 30% sucrose overnight followed by OCT embedding on dry ice. Embedded tissues were stored in −20° C. for at least 24 h prior to sectioning. Six micrometer (6 μM) frozen sections were cut using a cryotome and transferred onto superfrost plus microscope slides. Nuclear staining was achieved with a DAPI solution (1:250 dilution in PBS).

Tumor Immunofluorescence. Prior to processing, frozen sections were dried in the dark for 15 min. Slides were then washed three times with PBS, followed by 10 min incubation with permeabilization solution containing 0.3% triton X-100 and 30% sucrose. Following two PBS washes, tissue sections were blocked with 500 μL of blocking solution (10% normal goat serum) in a humidification chamber for 1 h at room temperature. In order to minimize drying, all slides were covered with Apoptag plastic coverslips (EMD Millipore) during incubations. Following blocking, tissue sections were incubated overnight at 4° C. with 350 μL of primary antibody diluted in blocking solution. Tissue sections were washed with PBS and incubated with secondary antibody for 1 h at room temperature. The following primary and secondary antibodies were used: mouse Alexa Fluor 647 anti-γH2A.X (pS139) (1:100; #560447; BD Bioscience, Franklin Lakes, N.J., USA), rabbit anti-HER2 (1:250; #4290; Cell Signaling Tech., Danvers, Mass., USA), anti-rabbit Alexa Fluor Plus 488 and anti-rabbit Alexa Fluor Plus 647 (1:400 each, #A32731 and #A21244 respectively; Thermo Fisher Scientific, Waltham, Mass., USA). Some samples were additionally stained for F-actin filaments (cytoplasmic marker) using fluorescein isothiocyanate labeled phalloidin (100 nM; #P5282; Burlington, Mass., USA). Nuclear staining was achieved with a DAPI solution (1:250 dilution in PBS). Subsequently, samples were washed three times with PBS, slides were mounted with glass coverslips using DAKO fluorescence Mounting Medium.
Microscopy Imaging and Quantification. Images were obtained with a Nikon Eclipse Ti fluorescence microscope with a Plan Apo 60×/1.40 Oil DIC h objective, a CSU-W1 confocal scanning unit with an iXon Ultra camera (Andor Technology, Belfast, UK), MLC 400B laser unit (Agilent Technologies, Santa Clara, Calif., USA), and NIS Elements 4.30 software (Nikon Corporation, Tokyo, Japan). Magnification for all images was 600×. Images were taken with three fourth of the maximum intensity without overexposure. The pictures were saved as a 16-bit Tagged Image File Format (TIFF), with no further editing. Representative images were generated using ImageJ version 1.52t (NIH, Bethesda, Md., USA). Whole sample and nuclear nanoparticles foci, along with γH2AX, cleaved caspase 3 and HER2 expression were analyzed with the Focinator v2-31 software as previously described³¹⁻³³. Software, instructions and supporting information are provided at focinator.com. TFO biodistribution within a designated set of tissues is reported as the TAMRA-HER2-205 fluorescence intensity for each tissue as a percentage of the combined total TAMRA-HER2-205 fluorescence intensities detected in the spleen, kidney, liver and tumor. TAMRA fluorescence intensity was quantified for each tissue using the Focinator as a measure of HER2-205 uptake. The sum of these fluorescence intensities within each organ (liver, spleen, kidney and tumor) yielded the total fluorescence or HER2-205 uptake and was depicted through the pie charts. Each slice gives the relative percent HER2-205 uptake for each organ.
Statistical analysis. Statistical analysis was performed by one-way or two-way ANOVA with the Tukey's test as post hoc unless otherwise stated. All analysis was completed using GraphPad Prism software. ****p<0.0001, ***p<0.001, **p<0.01, *p<0.05.

### Results

The present disclosure relates to a drug platform that converts (e.g., directly converts) amplified oncogenic driver genes into DNA damage to trigger cell death (FIG. 1A). This approach employs TFOs that recognize unique polypurine sites within the amplified chromosomal region¹⁴⁻¹⁶. Binding of TFOs within the major groove of the double helix causes DNA perturbation and double strand break (DSB) formation; for example, DNA perturbation can impede replication fork progression, resulting in fork collapse and DSB formation¹⁷. Formation of multiple chromosomal triplex structures can induces sufficient DNA damage to activate apoptosis in human cells¹⁸. The nucleotide excision repair (NER) pathway resolves low levels of triplex-induced DNA damage and hence normal cells can tolerate TFO treatment^{19,20}.

Amplified regions of a gene can span kilobases to tens of megabases that include multiple oncogenic genes, as well as passenger genes. Across the 14 human cancer subtypes characterized by gene amplification, the majority should have amplified regions with sequences that are conducive for a TFO approach, such as the present TFO approach.

For example, HER2 gene amplification in breast cancers provides an opportunity to test the efficacy of TFOs as a specific apoptosis-inducing agent in cancer cells,with limited toxicity in healthy cells, which lack HER2 amplification²¹ (FIGS. 1A-1C). This approach is particularly feasible due to the presence of several polypurine sites in the HER2 gene that are a prime target for triplex formation¹⁵.

### Targeting Gene Amplification in Cancer Via Triplex Formation

A TFO, HER2-1, was designed to target the polypurine sequence in the promoter region of the HER2 gene at positions −218 to −245, relative to the transcription start site (FIG. 1D). Another polypurine site favorable for high affinity triplex formation is located within the coding region beginning at position 205 and is targeted by TFO, HER2-205 (FIG. 1D). To confirm chromosomal TFO binding, non-denatured metaphase spreads were prepared from MCF7 and BT474 breast cancer cells that had been treated with TAMRA-labeled HER2-205. The generation of chromosomal HER2-205 foci represent third strand binding to fixed chromosomes with intact DNA double helix, indicative of triplex formation¹⁸. Using a FITC labeled satellite probe specific for human chromosome 17, gene-specific triplex formation was verified (FIG. 1E). TAMRA-HER2-205 chromosomal foci were only generated on chromosome 17, the location of the HER2 gene, thus validating target site specificity (FIG. 1E).

The Level of Triplex-Induced DNA Damage Correlates with Higher Gene Copy Numbers

Next, it was assessed whether the level of triplex-induced DNA damage correlated with higher gene copy numbers. A neutral comet assay, was used to establish that HER2-205 was more effective at inducing DNA damage than HER2-1, as indicated by an increase in DNA tail moment (FIG. 2A). Additionally, HER2-205 induced significantly more DSBs in cell lines containing multiple copies of the HER2 gene (FIGS. 2B and 7A). Results showed that the level of triplex-induced DNA damage was directly proportional to gene copy number (FIG. 2C). Markedly increased γH2AX positive cells, indicative of DSBs, were observed upon treatment of breast cancer cells with high HER2 gene copy numbers (FIG. 2D). 53BP1 foci was also assessed, which colocalizes with γH2AX at damage sites. HER2-205 treated BT474 cells exhibited substantially increased γH2AX and 53BP1 foci, compared to cells treated with the control oligonucleotide MIX24 (FIG. 2E). Furthermore, colocalization of γH2AX and 53BP1 was observed in 49% of cells following HER2-205 treatment (FIG. 7B).

Given the association of increased DNA damage with activation of apoptosis, it was hypothesized that HER2-targeting TFOs would be capable of inducing apoptosis specifically in amplified breast cancer cells. Assessment of effects of Her2-targeting TFOs demonstrated TFO-induced apoptosis specifically in the HER2-positive cell lines and that HER2-205 treatment resulted in a higher percentage of apoptotic cells than treatment with HER2-1 (FIGS. 2F, 2G, and 7C). Together, the results demonstrate that the intensity of triplex-induced DNA damage and apoptosis is dependent on—related to/correlated with gene copy number (FIGS. 2C, 2G). Furthermore, these results show that triplex-induced apoptosis provides the basis for novel therapeutics that specifically target cancers stemming from gene amplification, while sparing normal non-amplified tissues.

### HER2-205 Treatment Effectively Targets HER2 Positive Ovarian Cancers

To demonstrate the adaptability of this technology to target other cancers, therapeutic efficacy was evaluated in HER2-positive ovarian cancers. When administered to PEO1 and SKOV3 cells, both of which have HER2 copy number gains (FIG. 7D), HER2-205 treatment induced increased γH2AX foci and DNA tail moments (FIGS. 7E-7H). Elevated levels of unrepaired DSBs were s also observed in the untreated PEO1 cells, which harbor a deficiency in BRCA2, a key factor involved in DSB repair by homologous recombination (FIGS. 7G, 7H). Importantly, TFO treatment significantly increased the level of DSBs above baseline (FIG. 7H). In addition, HER2-205 reduced cell viability (FIG. 7I) and activated apoptosis in both cancer cell lines (FIGS. 2H and 7J).

Active HER2-Targeted TFO could Potentially be Used Clinically to Treat HER2-Positive Cancers

It was reasoned that the HER2-targeting TFO could have clinical efficacy in treating HER2-positive cancers. Therefore, two independent subcutaneous xenograft tumor models were developed to test this premise and confirmed TFO tumor uptake by ex vivo fluorescence imaging and confocal microscopy of tumor tissue using TAMRA-labeled HER2-205 (FIGS. 3A-3C). Importantly, treatment of BT474 human breast cancer tumors in athymic nude mice with HER2-205 suppressed tumor growth to a significantly greater degree than in the control group of animals treated with MIX24 (FIGS. 3D, 3F). IP administration of HER2-205 resulted in a notable reduction in tumor growth that was comparable to the currently used targeted therapy trastuzumab, thus demonstrating the utility of this gene-targeted cancer therapy (FIG. 3D, 3E). A tumor tripling time of 29±5.7 days post-initial dose was observed in tumors treated with HER2-205, compared to 24±2.1 days in tumors treated with trastuzumab (FIG. 3G). In contrast, the control oligonucleotide, MIX24 had no impact on BT474 tumor growth relative to the control buffer alone, with a tumor tripling time for control tumors of 15.7±4.9 days versus 16.3±6.6 days in tumors treated with MIX24 (ANOVA, p=0.99; FIG. 3G). Histological and immunohistochemical analyses were performed on paraffin-embedded tumor tissue sections. Tumor cell apoptosis (evidenced by the presence of cleaved caspase 3), decreased proliferation as measured by Ki67 staining and a confluent area of tumor necrosis were observed in the HER2-205 treated specimen (FIG. 3H). Magnification of the HER2-205 treated tumor revealed that areas of tumor cell apoptosis are accompanied by a brisk infiltrate of inflammatory cells consisting predominantly of neutrophils and macrophages (FIG. 3I).

The standard of care for epithelial ovarian cancers consists of platinum-based chemotherapy and surgical cytoreduction²². However, as in the case of the SKOV3 cell line, many human ovarian cancers are resistant to platinum-based drugs. Using SKOV3 ovarian cancer xenografts, it was found that HER2-205 treatment confers a substantial survival advantage compared with cisplatin (FIG. 3J). HER2-205 demonstrated significant tumor growth inhibitory activity; the average tumor volume was 49% smaller than that of tumors in cisplatin-treated mice (ANOVA, p=0.006). These data demonstrate the value of triplex-induced apoptosis as a therapeutic alternative for drug resistant cancers with copy number gains.

### Assessment of Versatility of the Approach

In humans, there is at least one unique and high affinity triplex targeting site located in the promoter and transcribed regions of each protein-coding gene. Mapping of polypurine sequences with characteristics to serve as a potential target site has identified 519,971 unique sequences throughout the human genome²³. In order to further investigate the versatility of the approach, TFOs were designed to target sites within other regions of the HER2 gene (FIG. 4A). HER2-5922 was designed to target a polypurine sequence in intron 2 and HER2-40118 was directed to a sequence within intron 19 (FIG. 4A). The TFOs were first assessed for their ability to induce DNA damage, compared to TFOs targeting the promoter and coding regions. It was determined by neutral comet assay that HER2-5922 and HER2-40118 were more effective at inducing DNA damage than HER2-205 as indicated by an increase in DNA tail moment (FIG. 4B). BT474 cells were then exposed to increasing concentrations of the HER2-targeted TFOs. As shown in FIG. 4C, cell viability decreased with increasing TFO concentrations, with HER2-205, HER2-5922 and HER2-40118 exhibiting similar dose responses with ˜50% cell death at a concentration of 12.5 nM. Western blot analysis of cleaved PARP confirmed triplex-induced apoptosis that corresponded to an increase in DSBs as indicated by H2AX phosphorylation at S139 (FIG. 4D). These results solidify the feasibility of this therapeutic strategy and emphasize that every amplified cancer driver gene should have multiple polypurine sequences that can be targeted using gene-specific bioactive TFOs.

Apoptosis Corresponds with the Phosphorylation of Specific DNA Damage Response Proteins and XPD is Required for the Induction of Apoptosis

To define the mechanism of drug action and characterize the DNA damage response activated in TFO-treated cells, target specific induction of DNA damage by HER2-205 was confirmed using chromatin immunoprecipitation (ChIP) assays for γH2AX and multiplexed qPCR with a probe for the HER2 gene locus. A 22-fold enrichment of γH2AX was detected at the HER2 gene relative to untreated cells 8 h post TFO-treatment (FIG. 5A). Moreover, analysis for the induction of DNA damage at a non-targeted region of the genome using a probe for the GAPDH gene locus did not detect the presence of γH2AX above background levels following HER2-205 treatment (FIG. 5A). Furthermore, targeting of the intron with HER2-5922 also resulted in gene-specific induction of DNA damage (FIG. 8A). These findings support a mechanism that TFO-generated structures can induce DNA damage specifically at the targeted amplified oncogenic gene locus.

Next, the status of ATM, Chk1/Chk2 and the NER factor, XPD in HER2 positive cells following HER2-205 treatment was determined. As shown in FIG. 5B, Chk1 phosphorylation at serine 345 was observed after HER2-205 treatment in the HER2-amplified cells and not in the cells with normal HER2 gene copy numbers. Chk1 activation in BT474 cells corresponds to induction of DSBs and apoptosis as determined by Western blot analysis of pH2AX S139 and cleaved PARP, respectively. In addition, phosphorylation of Chk2 at threonine 68 was observed in response to triplex-induced DSBs in the BT474 cells (FIG. 5B). These phosphorylation events correspond to an increase in pATM positive cells following HER2-205 treatment (FIG. 8B).

Regulation of the phosphorylation status of H2AX at tyrosine 142 (Y142) is crucial for determining the recruitment of either DNA repair or pro-apoptotic factors to the DSB site²⁴. It has been found that H2AX Y142 is phosphorylated in response to HER2-205 induced DSBs to trigger apoptosis as indicated by Western blot analysis of cleaved PARP (FIG. 5C). XPD occupies a central role in the mechanism that modulates survival/death decisions in response to triplex-induced DNA damage¹⁸. Accordingly, a requirement for XPD in the phosphorylation of Y142 in H2AX and activation of apoptosis following HER2-205 treatment was seen (FIG. 5C). These results suggest that the absence of XPD disrupts the signaling pathway used to activate apoptosis following TFO treatment and support a mechanism of action that is dependent upon DNA damage response.

The p53 tumor suppressor regulates pro-apoptotic pathways in response to severe DNA damage. However, over 50% of human cancers exhibit chemotherapeutic resistant phenotypes due to loss of function p53 mutations which lead to an inability to trigger apoptosis.

HER2-205 Treatment Activates p53-Independent Apoptosis

To test whether triplex-induced DNA damage could activate p53-independent apoptosis, p53-depleted BT474 cells were treated with HER2-205. It was found that TFO-treatment of p53-depleted cells results in a similar level of PARP cleavage compared to treatment of control cells, confirming that triplex formation can activate apoptosis irrespective of p53 status (FIG. 5D). Unlike XPD-depleted cells, which displayed a decrease in TFO-induced apoptosis, it was also demonstrated that triplex-induced DSBs trigger robust H2AX Y142 phosphorylation in the absence of p53 (FIGS. 5C, 5D).

### HER2-Targeted TFO Treatment is Independent of HER 2 Gene Expression and Cellular Function

Trastuzumab's anticancer activity has been attributed in part to changes in HER2 tyrosine phosphorylation and a reduction in total HER2 protein^{25,26}. To further demonstrate that HER2-205 activity is independent of the cellular function of HER2, HER2 gene expression was analyzed by RT-PCR (FIG. 5E) and monitored total HER2 protein and phosphorylation levels by Western blot following treatment in several breast cancer cell lines (FIG. 5F). The results showed that HER2 gene expression is not significantly affected by HER2-205 treatment in either the non-amplified or amplified breast cancer cell lines (FIG. 5E) and that total and activated HER2 levels remain the same following triplex-induced apoptosis in the HER2-positive cells compared to the control samples (FIG. 5F). Although a significant impact on HER2 gene expression following HER2-1 treatment was not detected, significantly more TFO-induced DNA damage and activation of apoptosis with HER2-205 was observed compared to HER2-1, further supporting a mechanism that activity is dependent on the ability to induce DNA damage and not disruption of gene expression (FIGS. 8C-8E). In general, no changes were noted in the levels of HER3, HER4 and EGFR following HER2-205 treatment compared to the untreated or MIX24 treated cells (FIGS. 8F-8H). Additionally, targeting in the introns did not result in a significant decrease in HER2 gene expression following treatment with either HER2-5922 or HER2-40118 (FIG. 8I).

In order to investigate whether active transcription was a prerequisite for TFO induced DNA damage and activation of apoptosis, transcription was inhibited in BT474 cells with α-amanitin prior to HER2-205 treatment (FIG. 8J). The results demonstrate similar increases in the levels of γH2AX and cleaved caspase 3 compared to controls following TFO treatment in cells with and without transcription inhibitor pretreatment (FIG. 5G), suggesting that active transcription is not required for TFO-induced DNA damage or activation of apoptosis. Taken together, these results support a mechanism of action for the HER2-targeted TFO that is independent of the cellular function of HER2 (FIG. 5H).

Inadequate drug delivery to the intended disease site can significantly impact therapeutic effect. It was hypothesized that polymeric, biodegradable nanoparticles (NPs) could serve as a delivery platform and enhance drug efficacy of the HER2-targeted TFOs. Several NPs formulations were evaluated for their tumor delivery potential by screening for fluorescent dye uptake using DiD (FIGS. 9A-9B). DiD was encapsulated using two NP formulations fabricated from poly(lactic-co-glycolic acid) (PLGA), a polymer that has been approved by the FDA for numerous drug delivery applications. In addition, NPs were screened from a copolymer of poly(lactic acid) (PLA) and hyperbranched polyglycerol (HPG), PLA-HPG, which has previously demonstrated long blood circulation times and effective tumor uptake²⁷. It was determined that DiD-loaded PLA-HPG NPs had more efficient tumor uptake compared to the PLGA formulations following intravenous administration by retro-orbital (RO) injection in an orthotopic mouse model of BT474 breast cancer cells (FIG. 9A). To better understand the overall impact of PLA-HPG NPs delivery, biodistribution in the liver, lung, spleen, kidney and heart was evaluated 12 h post administration (FIG. 9B).

To visualize TFO uptake and inform timing and dosing for subsequent tumor growth studies, TAMRA-HER2-205 was encapsulated in PLA-HPG NPs and tumor distribution was evaluated 12 and 24 hours post systemic administration in mice bearing orthotopic BT474 breast cancer tumors (FIG. 6A). To confirm that cells internalized HER2-205, confocal microscopy was performed on tumor tissue and quantified nuclear and extranuclear TAMRA fluorescence. Administration of the NPs resulted in TAMRA-HER2-205 accumulation in the tumor, which is detectable at 12 hours with a slight reduction in fluorescence intensity 24 hours post treatment (FIG. 6B). Furthermore, PLA-HPG NPs delivered TAMRA-HER2-205 to the nucleus, with significantly greater fluorescence being detected at 12 h post-treatment compared to 24 h (FIG. 6B). The fluorescence intensity of TAMRA-HER2-205 in the liver, kidney and spleen compared to tumors was also evaluated in order to determine TFO biodistribution at 12 and 24 hours post treatment (FIGS. 10A-10E). Administration of TAMRA-HER2-205 PLA-HPG NPs resulted in ˜10% distribution of the TFO in the tumor at both time points (FIGS. 10D, 10E). As observed in the biodistribution studies using DiD, higher uptake was detected in the liver and spleen (27% and 49%, respectively at 12 hours post-treatment) compared to the tumor (FIGS. 10D, 10E).

The impact of the PLA-HPG delivery system (FIG. 11A) on therapeutic efficacy was assessed, using an orthotopic mouse tumor xenograft model of BT474 breast cancer cells. In this model, systemic administration of HER2-205 and HER2-5922 PLA-HPG NPs significantly decreased tumor growth and improved survival (as defined by tumor volume 3× that of initial treatment) (FIGS. 6C, 6D). As observed in other models, MIX24 PLA-HPG NPs did not alter tumor growth or survival as compared with untreated controls (FIG. 6C). The efficacy of this therapeutic strategy is not limited by the target region; HER2-5922 NP treatment resulted in a significant delay in tumor growth (FIG. 6C). A tumor tripling time of 45±1.5 days post-initial dose was observed in tumors treated with HER2-205 NPs and 47±1.2 days with HER2-5922 NPs compared to 33±1.4 days with MIX24 NPs (FIG. 6D). In contrast, MIX24 NPs had no impact on BT474 tumor growth relative to untreated controls (31±0.8 2 days). Notably, when compared to treatment with naked oligonucleotides, the TFO encapsulated PLA-HPG NP delivery system reduced by more than 99% the amount of TFO (˜80 nmol/dose with naked TFO compared to −0.14 nmol/dose with NPs) required to have a significant impact on tumor growth delay and improved survival. Additionally, no gross toxicity, including weight loss was noted in mice treated with the HER2-targeted TFOs NPs (FIG. 6E).

To investigate the mechanism underlying efficacy, BT474 tumor-bearing mice were treated with HER2-205 NPs (2 mg, RO injection) and collected tumors for immunofluorescence analysis 12 and 24 hours after treatment. As compared with untreated controls, tumors treated with HER2-205 had significantly higher levels of γH2AX foci (2-fold increase as measured by MFI), confirming that HER2-targeted TFOs were capable of inducing DNA damage in tumors (FIGS. 6F and 11B)). To determine whether this degree of DNA damage was sufficient to induce cell death, the activation of caspase 3 as a biomarker for apoptosis was also investigated (FIGS. 6G and 11C)). A significant increase in apoptosis was observed 24 h following HER2-205 treatment compared to untreated tumors (FIG. 6G), which corresponded to the increase in DNA damage observed at 12 h (FIG. 6F). Importantly, similar to the mechanistic studies in cells, a decrease in HER2 protein levels was not observed in the HER2-205 treated tumors compared to tumors from untreated mice (FIGS. 11D, 11E). Together, these results indicate that HER2-205 exhibits therapeutic efficacy utilizing a mechanism independent of HER2 cellular function and based on DNA damage response, where induction of DNA damage activates apoptosis.

### Other Embodiments

All features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

### REFERENCES

1 Mc, C. B. Chromosome organization and genic expression. Cold Spring Harb Symp Quant Biol 16, 13-47 (1951).
2 Matsui, A., Ihara, T., Suda, H., Mikami, H. & Semba, K. Gene amplification: mechanisms and involvement in cancer. Biomol Concepts 4, 567-582, doi:10.1515/bmc-2013-0026 (2013).
3 Santarius, T., Shipley, J., Brewer, D., Stratton, M. R. & Cooper, C. S. A census of amplified and overexpressed human cancer genes. Nat Rev Cancer 10, 59-64, doi:10.1038/nrc2771 (2010).
4 Albertson, D. G. Gene amplification in cancer. Trends Genet 22, 447-455, doi:10.1016/j.tig.2006.06.007 (2006).
5 Moasser, M. M. & Krop, I. E. The Evolving Landscape of HER2 Targeting in Breast Cancer. JAMA Oncol 1, 1154-1161, doi:10.1001/jamaonco1.2015.2286 (2015).
6 Swain, S. M. et al. Pertuzumab, trastuzumab, and docetaxel in HER2-positive metastatic breast cancer. The New England journal of medicine 372, 724-734, doi:10.1056/NEJMoa1413513 (2015).
7 Wilks, S. T. Potential of overcoming resistance to HER2-targeted therapies through the PI3K/Akt/mTOR pathway. Breast 24, 548-555, doi:10.1016/j.breast.2015.06.002 (2015).
8 Chen, Y. et al. Identification of druggable cancer driver genes amplified across TCGA datasets. PloS one 9, e98293, doi:10.1371/journal.pone.0098293 (2014).
9 Ohshima, K. et al. Integrated analysis of gene expression and copy number identified potential cancer driver genes with amplification-dependent overexpression in 1,454 solid tumors. Sci Rep 7, 641, doi:10.1038/s41598-017-00219-3 (2017).
10 Slamon, D. J. et al. Studies of the HER-2/neu proto-oncogene in human breast and ovarian cancer. Science (New York, N.Y 244, 707-712 (1989).
11 Baselga, J., Albanell, J., Molina, M. A. & Arribas, J. Mechanism of action of trastuzumab and scientific update. Seminars in oncology 28, 4-11 (2001).
12 Petty, R. D. et al. Gefitinib and EGFR Gene Copy Number Aberrations in Esophageal Cancer. J Clin Oncol 35, 2279-2287, doi:10.1200/JCO.2016.70.3934 (2017).
13 Pao, W. et al. Acquired resistance of lung adenocarcinomas to gefitinib or erlotinib is associated with a second mutation in the EGFR kinase domain. PLoS Med 2, e73, doi:10.1371/journal.pmed.0020073 (2005).
14 Ricciardi, A. S., McNeer, N. A., Anandalingam, K. K., Saltzman, W. M. & Glazer, P. M. Targeted genome modification via triple helix formation. Methods Mol Biol 1176, 89-106, doi:10.1007/978-1-4939-0992-6_8 (2014).
15 Gaddis, S. S. et al. A web-based search engine for triplex-forming oligonucleotide target sequences. Oligonucleotides 16, 196-201 (2006).
16 Ebbinghaus, S. W. et al. Triplex formation inhibits HER-2/neu transcription in vitro. The Journal of clinical investigation 92, 2433-2439 (1993).
17 Kaushik Tiwari, M., Adaku, N., Peart, N. & Rogers, F. A. Triplex structures induce DNA double strand breaks via replication fork collapse in NER deficient cells. Nucleic acids research 44, 7742-7754, doi:10.1093/nar/gkw515 (2016).
18 Kaushik Tiwari, M. & Rogers, F. A. XPD-dependent activation of apoptosis in response to triplex-induced DNA damage. Nucleic acids research 41, 8979-8994, doi:10.1093/nar/gkt670 (2013).
19 Rogers, F. A., Vasquez, K. M., Egholm, M. & Glazer, P. M. Site-directed recombination via bifunctional PNA-DNA conjugates. Proceedings of the National Academy of Sciences of the United States of America 99, 16695-16700 (2002).
20 Wang, G., Seidman, M. M. & Glazer, P. M. Mutagenesis in mammalian cells induced by triple helix formation and transcription-coupled repair. Science (New York, N.Y 271, 802-805 (1996).
21 Szollosi, J., Balazs, M., Feuerstein, B. G., Benz, C. C. & Waldman, F. M. ERBB-2 (HER2/neu) gene copy number, p185HER-2 overexpression, and intratumor heterogeneity in human breast cancer. Cancer research 55, 5400-5407 (1995).
22 Vergote, I. et al. Neoadjuvant chemotherapy or primary surgery in stage IIIC or IV ovarian cancer. The New England journal of medicine 363, 943-953, doi:10.1056/NEJMoa0908806 (2010).
23 Jenjaroenpun, P. & Kuznetsov, V. A. TTS mapping: integrative WEB tool for analysis of triplex formation target DNA sequences, G-quadruplets and non-protein coding regulatory DNA elements in the human genome. BMC Genomics 10 Suppl 3, S9, doi:10.1186/1471-2164-10-S3-S9 (2009).
24 Cook, P. J. et al. Tyrosine dephosphorylation of H2AX modulates apoptosis and survival decisions. Nature 458, 591-596, doi:10.1038/nature07849 (2009).
25 zum Buschenfelde, C. M., Hermann, C., Schmidt, B., Peschel, C. & Bernhard, H. Antihuman epidermal growth factor receptor 2 (HER2) monoclonal antibody trastuzumab enhances cytolytic activity of class I-restricted HER2-specific T lymphocytes against HER2-overexpressing tumor cells. Cancer research 62, 2244-2247 (2002).
26 Cuello, M. et al. Down-regulation of the erbB-2 receptor by trastuzumab (herceptin) enhances tumor necrosis factor-related apoptosis-inducing ligand-mediated apoptosis in breast and ovarian cancer cell lines that overexpress erbB-2. Cancer research 61, 4892-4900 (2001).
27 Deng, Y. et al. The effect of hyperbranched polyglycerol coatings on drug delivery using degradable polymer nanoparticles. Biomaterials 35, 6595-6602, doi:10.1016/j.biomaterials.2014.04.038 (2014).
28 Bindra, R. S. & Glazer, P. M. Repression of RAD51 gene expression by E2F4/p130 complexes in hypoxia. Oncogene 26, 2048-2057, doi:10.1038/sj.onc.1210001 (2007).
29 Balashanmugam, M. V. et al. Preparation and characterization of novel PBAE/PLGA polymer blend microparticles for DNA vaccine delivery. ScientificWorldJoumal 2014, 385135, doi:10.1155/2014/385135 (2014).
30 Seo, Y. E. et al. Nanoparticle-mediated intratumoral inhibition of miR-21 for improved survival in glioblastoma. Biomaterials 201, 87-98, doi:10.1016/j.biomaterials.2019.02.016 (2019).
31 Oeck, S. et al. The Focinator v2-0—Graphical Interface, Four Channels, Colocalization Analysis and Cell Phase Identification. Radiat Res 188, 114-120, doi:10.1667/RR14746.1 (2017).
32 Oeck, S., Malewicz, N. M., Hurst, S., Rudner, J. & Jendrossek, V. The Focinator—a new open-source tool for high-throughput foci evaluation of DNA damage. Radiat Oncol 10, 163, doi:10.1186/s13014-015-0453-1 (2015).
33 Mandl, H. K. et al. Optimizing biodegradable nanoparticle size for tissue-specific delivery. Journal of controlled release: official journal of the Controlled Release Society 314, 92-101, doi:10.1016/j.jconrel.2019.09.020 (2019).

What is claimed is:

1. A method of inducing target-specific DNA damage in cancer cells in which a gene is amplified, the method comprising contacting (a) cancer cells in which a gene is amplified with (b) triplex forming oligonucleotides (TFOs) targeted to a polypurine site, referred to as a targeted amplified gene locus, within the amplified gene,
under conditions under which the TFOs enter the cancer cells and induce DNA damage at the targeted amplified gene locus.

2. The method of claim 1, wherein the amplified gene is a cancer-promoting gene and DNA damage is copy number dependent.

3. The method of claim 2, wherein the DNA damage is double strand breaks.

4. The method of claim 2, wherein the cancer-promoting gene is linked to a signaling pathway.

5. The method of claim 2, wherein DNA damage is sufficient to activate apoptosis in the cancer cells.

6. The method of claim 2, wherein the amplified cancer-promoting gene is HER2 and the cancer cells are HER2-amplified breast cancer cells or HER2-amplified ovarian cancer cells.

7. The method of claim 4, wherein the polypurine target site is/comprises SEQ ID NO: 1, SEQ ID NO: 2, SEQ ID NO: 5 or SEQ ID NO: 6.

8. The method of claim 4, wherein the TFOs comprise at least one of the following: a nucleotide sequence identical to SEQ ID NO:3; a nucleotide sequence identical to SEQ ID NO:4; a nucleotide sequence identical to SEQ ID NO:7; a nucleotide sequence identical to SEQ ID NO:8; a nucleotide at least 90% identical to SEQ ID NO: 3; a nucleotide sequence at least 90% identical to SEQ ID NO: 4; a nucleotide sequence at least 90% identical to SEQ ID NO: 7 and a nucleotide at least 90% identical to SEQ ID NO: 8.

9. The method of claim 1, wherein the polypurine site is in (a) the promoter region of the amplified cancer-promoting gene; (b) a coding region of the amplified cancer-promoting gene; or (c) an intron of the amplified cancer-promoting gene.

10. A method of reducing, in a population of cells comprising cancer cells, the number of cancer cells in which a cancer-promoting gene is amplified, the method comprising contacting the population of cells with triplex forming oligonucleotides (TFOs) targeted to a polypurine target site in the amplified cancer-promoting gene, under conditions under which the TFOs enter the cancer cells in sufficient quantity to induce apoptosis.

11. The method of claim 10, wherein the population of cells is in an individual.

12. The method of claim 10, wherein the polypurine target site is in (a) the promoter region of the amplified cancer-promoting gene; (b) a coding region of the amplified cancer-promoting gene; or (c) an intron of the amplified cancer-promoting gene.

13. A method of activating apoptosis in cancer cells that comprise an amplified cancer-promoting gene, comprising contacting cancer cells comprising an amplified cancer-promoting gene with triplex forming oligonucleotides (TFOs) targeted to a polypurine site within the amplified cancer-promoting gene, under conditions under which the TFOs enter the cancer cells in sufficient quantity to induce copy number dependent DNA damage and activate apoptosis.

14. The method of claim 13, wherein the amplified cancer-promoting gene is HER2, the cancer cells are HER2-amplified breast cancer cells or HER2-amplified ovarian cancer cells and the TFOs comprise at least one of the following: a nucleotide sequence identical to SEQ ID NO:3; a nucleotide sequence identical to SEQ ID NO:4; a nucleotide sequence identical to SEQ ID NO:7; a nucleotide sequence identical to SEQ ID NO:8; a nucleotide at least 90% identical to SEQ ID NO: 3; a nucleotide sequence at least 90% identical to SEQ ID NO: 4; a nucleotide sequence at least 90% identical to SEQ ID NO: 7 and a nucleotide at least 90% identical to SEQ ID NO: 8.

15. The method of claim 14, wherein the cancer cells are in an individual.

16. A composition comprising: triplex forming oligonucleotides (TFOs) and poly (lactic acid)-hyperbranched polyglycerol (PLA-HPG) polymer nanoparticles.

17. The composition of claim 16, wherein the TFOs comprise at least one of the following: a nucleotide sequence identical to SEQ ID NO:3; a nucleotide sequence identical to SEQ ID NO:4; a nucleotide sequence identical to SEQ ID NO:7; a nucleotide sequence identical to SEQ ID NO:8; a nucleotide at least 90% identical to SEQ ID NO: 3; a nucleotide sequence at least 90% identical to SEQ ID NO: 4; a nucleotide sequence at least 90% identical to SEQ ID NO: 7 and a nucleotide at least 90% identical to SEQ ID NO: 8.

18. A method of administering triplex forming oligonucleotides (TFOs) to an individual, comprising administering a composition of claim 16 to an individual by intravenous injection.


SEQUENCE LISTING (17 sequences)

SEQ ID NO: 1 - DNA, 28 bp, Artificial Sequence
  Synthetic Polynucleotide
aggagaaggaggaggtggaggaggaggg

SEQ ID NO: 2 - DNA, 28 bp, Artificial Sequence
  Synthetic Polynucleotide
ggggggcaagagggcgaggaggagcccc

SEQ ID NO: 3 - DNA, 28 bp, Artificial Sequence
  Synthetic Polynucleotide
gggaggaggaggtggaggaggaagagga

SEQ ID NO: 4 - DNA, 24 bp, Artificial Sequence
  Synthetic Polynucleotide
gaggaggagtgggagaatgggggg

SEQ ID NO: 5 - DNA, 30 bp, Artificial Sequence
  Synthetic Polynucleotide
ggggtgaggagagtgggggaggagaaaggg

SEQ ID NO: 6 - DNA, 22 bp, Artificial Sequence
  Synthetic Polynucleotide
ggggtgggagggacaaaggggg

SEQ ID NO: 7 - DNA, 30 bp, Artificial Sequence
  Synthetic Polynucleotide
gggaaagaggagggggtgagaggagtgggg

SEQ ID NO: 8 - DNA, 22 bp, Artificial Sequence
  Synthetic Polynucleotide
gggggaaatagggagggtgggg

SEQ ID NO: 9 - DNA, 24 bp, Artificial Sequence
  Synthetic Polynucleotide
agtcagtcagtcagtcagtcagtc

SEQ ID NO: 10 - DNA, 28 bp, Artificial Sequence
  Synthetic Polynucleotide
ccctcctcctccacctcctccttctcct

SEQ ID NO: 11 - DNA, 28 bp, Artificial Sequence
  Synthetic Polynucleotide
ggggctcctcctcgccctcttgcccccc

SEQ ID NO: 12 - DNA, 30 bp, Artificial Sequence
  Synthetic Polynucleotide
ccctttctcctcccccactctcctcacccc

SEQ ID NO: 13 - DNA, 22 bp, Artificial Sequence
  Synthetic Polynucleotide
ccccctttgtccctcccacccc

SEQ ID NO: 14 - DNA, 19 bp, Artificial Sequence
  Synthetic Polynucleotide
gacagtcgagacgctcagg

SEQ ID NO: 15 - DNA, 20 bp, Artificial Sequence
  Synthetic Polynucleotide
ggaaagcgccagtctcttgg

SEQ ID NO: 16 - DNA, 21 bp, Artificial Sequence
  Synthetic Polynucleotide
gccttgtagctaaggatcacc

SEQ ID NO: 17 - DNA, 21 bp, Artificial Sequence
  Synthetic Polynucleotide
caacccccaggacgaaaaaag